(19) United States
(12) Patent Application Publication
(10) Pub. No.: US 2003/0180740 A1
(43) Pub. Date: Sep. 25, 2003

(54) Differential expression screening method

(76) Inventor: Alan John Kingsman, Oxford (GB)

Correspondence Address: Bruce D Grant, Morrison & Foerster, Suite 500, 3811 Valley Centre Drive, San Diego, CA, 92130-2332, US

(21) Appl. No.: 10/204,724

(22) PCT Filed: Feb. 22, 2001
(86) PCT No.: PCT/GB01/00758

(30) Foreign Application Priority Data
Feb. 22, 2000 (GB) 0004197.0
Jul. 28, 2000 (GB) 0018679.1

(51) Int. Cl. C12Q 1/68

(52) U.S. Cl. 435/6

(57) ABSTRACT

A differential expression screening method is provided for identifying a genetic element involved in a cellular process, which method comprises comparing:

(a) gene expression in a first cell of interest; and

(b) gene expression in a second cell of interest, which cell comprises altered levels, relative to physiological levels, of a biological molecule implicated in the cellular process, due to the introduction into the second cell of a heterologous nucleic acid directing expression of a polypeptide; and

identifying a genetic element whose expression differs, wherein gene expression in said first and/or second cell of interest is compared under at least two different environmental conditions relevant to the cellular process.

### CROSS-REFERENCE TO RELATED APPLICATIONS

This application is a national phase of PCT/GB01/00758, filed 22 Feb. 2001, which claims priority over GB0018679.1, filed 28 Jul. 2000 and GB0004197.0, filed 22 Feb. 2000, and are incorporated in their entirety by reference.

### FIELD OF THE INVENTION

The present invention relates to methods of screening for genes by differential expression.

### BACKGROUND TO THE INVENTION

One of the central goals in the field of gene discovery is to understand and elucidate the relationship between a particular disease state and the gene expression pattern that defines and/or causes this disease state. In this way it is possible to identify genes which potentially are of great medical importance, either for the diagnosis or for the treatment of disease. The products of such genes may be useful directly as therapeutics, the genes themselves may be applicable to gene therapy, or small molecule effectors may be found to modulate the expression or the effects of these genes to treat disease. Research has concentrated on differences in expression patterns between diseased and healthy tissues to elucidate the physiological mechanisms of disease. Identified differences in expression patterns provide putative points for therapeutic intervention to reverse the disease phenotype. These differences also provide markers that are useful for diagnosis, and identify proteins for further investigation as agents implicated in the disease in question.

Differential screening of gene expression is one technique well known in the art which, often together with subtractive cDNA cloning methods, has been used successfully to identify genes involved in a range of cellular processes. Differential screening is generally performed using either a nucleic acid-based method where levels of mRNA expression are determined, or using a proteomics approach where the total protein content of a cell is resolved using techniques such as 2D gel electrophoresis.

One of the problems of the differential screening methods known to date, even those based on DNA chip technology, is that absolute levels of a gene product of interest, and/or the difference in expression of that gene product between two particular states (for example, in the presence and absence of a growth factor or in two different cell types) may be rather low. Consequently, although some very important genes have been identified to date using standard differential expression screening techniques, many genes that may play important roles in cellular processes are difficult to identify because their expression levels are low or because observable changes in their expression levels may be relatively small.

A further problem suffered by conventional methods of differential screening is that these methods do not allow dissection of the genetic or biochemical pathway that is being studied. Any changes in gene expression that are identified are global, rather than specific to a particular aspect of the pathway under investigation. There is thus a need in the art for a method that would facilitate the molecular dissection of biological pathways.

### SUMMARY OF THE INVENTION

It is therefore an object of the present invention to provide an improved screening method based on differential expression.

In a first aspect of the invention, a differential expression screening method is provided for identifying a genetic element involved in a cellular process which method comprises comparing gene expression in:

(a) a first cell of interest; and

(b) a second cell of interest which cell comprises altered levels, relative to physiological levels, of a biological molecule, due to the introduction into the second cell of a heterologous nucleic acid; and

identifying a genetic element whose expression differs.

The term “genetic element” is meant to include genes, gene products (such as RNA molecules, and polypeptides), cis-acting regulatory elements (such as promoter elements and enhancer elements). The method allows differences in the patterns of expression of any of these molecule types to be evaluated, and put into a biological context in the light of the cellular process that is being studied. The method also allows differences in the constituent genetic elements to be investigated, for example, to identify mutations and polymorphisms that affect the biological response to a particular cellular process.

In one embodiment, the first cell of interest also comprises altered levels, relative to physiological levels, of the biological molecule. However, in an alternative embodiment the first cell of interest has normal physiological levels of the biological molecule. The biological molecule may be functionally characterised, or not fully characterised.

Typically, in the second cell of interest, the levels of the biological molecule are enhanced or reduced. In a preferred embodiment, the biological molecule and the polypeptide encoded by the heterologous nucleic acid are the same molecule. The polypeptide may be functionally characterised, or not fully characterised.

Preferably, the nucleic acid directs expression of a polypeptide. Preferably, a polypeptide encoded by the heterologous nucleic acid is involved in the cellular process. By “involved in the cellular process” is meant that the gene has been found to possess a distinct role in a genetic or metabolic pathway in a cell. The polypeptide may be involved in susceptibility to, generation of, or maintenance of a particular disease phenotype or physiological condition. As will be apparent to the skilled reader, any point in any pathway may be the unique point at which a cell departs from the normal physiological response and generates a disease phenotype. Often the effect that is manifested as a disease is the result of a mutation event, in which a mutation occurs in the sequence of a gene encoding a protein that functions in a relevant physiological pathway.

Preferably, the nucleic acid is delivered to the cell using a viral vector. In this case, the heterologous nucleic acid should be co-linear with a viral vector. As the skilled reader will appreciate, different viral vectors are appropriate for various cell types. Preferred viral vectors for use in accordance with the present invention are derived from retroviruses, lentiviruses, such as the Equine Infectious Anaemia Virus (EIAV) or human immunodeficiency virus, type 1 (HIV-1), adenoviruses, adeno-associated viruses, herpes virus and pox viruses such as entomopox.

Preferred features of viral vectors for the purpose of the present invention are the ability efficiently to transduce the target cells, and the ability to minimise any perturbations in gene expression which may result from the use of the viral vector per se but which hare unrelated specifically to the introduction of the heterologous nucleic acid of interest (“phenotypic silence”). As will be appreciated by those skilled in the art of viral-mediated gene transfer, this the field is advancing rapidly, and preferred vectors for various cell types are changing as the field advances. For example, at the time of writing, the preferred vector for the transduction of macrophages is an adenoviral vector, because it enabled the highest possible level of transduction. This vector does not enable phenotypically silent transduction, but it is possible to exclude vector effects on cellular gene expression using appropriate controls. On the other hand, a vector derived from the lentivirus EIAV, which enables phenotypically silent transduction, gives the best available transduction in hippocampal neurones, and so is the vector of choice for that application. Phenotypic silence of the vector is always desirable, but must be balanced by transduction efficiency. The vector development described in the Examples included herein has been directed at the optimisation of these two features in the cell types described. As will be clear to those skilled in the art of vector technology, the present invention is independent of vector type, but its practice may be enhanced by the optimum choice of vector for each cell type.

Generally, gene expression in the first and second cell may be determined by using proteomic techniques, or by using nucleic acid-based genomic or cDNA techniques.

In a preferred embodiment of the first aspect of the invention, a differential expression screening method is provided for identifying a genetic element involved in a cellular process which method comprises comparing gene expression in:

(a) a first cell of interest; and

(b) a second cell of interest, which is different from the first cell and which cell comprises altered levels, relative to physiological levels, of a biological molecule, due to the introduction into the second cell of a heterologous nucleic acid; and

identifying a genetic element whose expression differs.

Preferably, the nucleic acid directs expression of a polypeptide, for example, a polypeptide involved in a cellular process, as discussed above.

In a second aspect, the present invention provides a differential expression screening method for identifying a genetic element whose expression is regulated by a signal, which method comprises comparing at two different levels of the signal:

(a) gene expression in a first cell of interest, wherein the signal is at a first level; and

(b) gene expression in a second cell of interest, which cell comprises altered levels, relative to physiological levels, of a biological molecule whose activity is responsive to the signal, due to the introduction into the second cell of a heterologous nucleic acid directing expression of a polypeptide, wherein the signal is at a second level; and

identifying a genetic element whose expression differs.

In a third aspect of the present invention, a polypeptide which is known or suspected to be involved in a cellular process is used to identify other components of the same process by altering the levels of that polypeptide in a cell to produce an improved signal to noise ratio for the levels of those other components to be identified, making them easier to identify by differential expression techniques.

Accordingly, the present invention also provides a differential expression screening method for identifying a genetic element whose expression is altered in a cellular process which method comprises comparing:

(a) gene expression in a first-cell of interest; and

(b) gene expression in a second cell of interest, which cell has been modified to contain altered levels of a polypeptide implicated in the cellular process; and

identifying a genetic element whose expression differs.

Preferably, the altered levels of the polypeptide are due to the introduction into the cell of a heterologous nucleic acid which directs the expression of the polypeptide in the cell. More preferably, the heterologous nucleic acid is colinear with a viral vector.

In a preferred embodiment of the third aspect of the invention, the expression of the genetic element is regulated by a biological signal, and the method includes the steps of comparing gene expression in the two cell types at two different levels of the signal.

This aspect of the invention therefore provides a differential expression screening method for identifying a genetic element involved in a cellular process, which method comprises comparing:

(a) gene expression in a first cell of interest; and

(b) gene expression in a second cell of interest, which cell comprises altered levels, relative to physiological levels, of a biological molecule implicated in the cellular process, due to the introduction into the second cell of a heterologous nucleic acid directing expression of a polypeptide; and

identifying a genetic element whose expression differs, wherein gene expression in said first and/or second cell of interest is compared under at least two different environmental conditions relevant to the cellular process. Preferably, gene expression is compared in both the first and the second cell of interest under at least two different environmental conditions relevant to the cellular process.

The environmental conditions to which the cells are exposed may, in one example, be different levels of a biological signal. Gene expression in the two cell types may be compared under environmental conditions in which the signal is absent, is present at a first level, and/or is present at a second level (for example, different percentages of atmospheric oxygen content between normoxia [20% oxygen] and hypoxia [<1% oxygen]). The use of at least two levels of a biological signal permits the comparison of the effects of the change in environmental conditions and of the heterologous nucleic acid on those cell types, and the identification of genetic elements whose expression behaves in the same way, or in different ways, between the levels of biological signal and environmental conditions tested. Of course, more than two levels of a biological signal can be applied in the same manner with different types of environmental change, cell type and heterologous nucleic acid.

One embodiment of this aspect of the invention therefore provides a differential expression screening method for identifying a genetic element involved in a cellular process, which method comprises comparing:

(a) gene expression in a first cell of interest;

(b) gene expression in the first cell of interest which has been exposed to a biological signal relevant to the cellular process, wherein the biological signal is at a first level;

(c) gene expression in the first cell of interest which has been exposed to a biological signal relevant to the cellular process, wherein the biological signal is at a second level; and

(d) gene expression in a second cell of interest, which cell comprises altered levels, relative to physiological levels, of a biological molecule whose activity is responsive to the biological signal, due to the introduction into the second cell of a heterologous nucleic acid directing expression of a polypeptide, wherein the signal is absent, at a first level or at a second level; and

identifying a genetic element whose expression differs.

In an alternative embodiment of this aspect of the invention, the environmental conditions to which the cells are exposed may be different types of environmental change (for example, changes in the levels of different growth factors to which the cells are exposed). The use of two environmental changes permits the comparison of the effects of each environmental change and of the heterologous nucleic acid on each cell type, and the identification of genetic elements whose expression behaves in the same way, or in different ways, between those environmental changes tested. More than two environmental changes can be applied in the same manner with each cell type and each heterologous nucleic acid.

This aspect of the invention thus provides a differential expression screening method for identifying a genetic element involved in a cellular process, which method comprises comparing:

(a) gene expression in a first cell of interest;

(b) gene expression in the first cell of interest which has been exposed to an environmental change of a first type;

(c) gene expression in the first cell of interest which has been exposed to an environmental change of a second type; and

(d) gene expression in a second cell of interest, which cell contains altered levels, relative to physiological levels, of a biological molecule whose activity is responsive to one or both of the environmental changes recited in parts b) and c), due to the introduction into the second cell of a heterologous nucleic acid directing expression of a polypeptide, under conditions in which the cell either has or has not been exposed to the first and/or the second type of environmental change; and

identifying a genetic element whose expression differs.

In the above embodiments of the invention, the first cell may also comprise altered levels, relative to physiological levels, of a biological molecule whose activity is responsive to the difference between the environmental conditions, due to the introduction into the cell of a heterologous nucleic acid directing expression of a polypeptide.

The biological molecule in the first cell may be the same biological molecule as that biological molecule whose levels are altered in the second cell. In this embodiment, the levels of the biological molecule in the first and second cells should be different.

This aspect of the invention thus provides a differential expression screening method for identifying a genetic element involved in a cellular process, which method comprises comparing:

(a) gene expression in a first cell of interest;

(b) gene expression in the first cell of interest, wherein the cell has been exposed to a biological signal relevant to the cellular process;

(c) gene expression in the first cell of interest, which cell contains altered levels, relative to physiological levels, of a biological molecule whose activity is responsive to the biological signal, due to the introduction into the first cell of a heterologous nucleic acid directing expression of a polypeptide, wherein the altered level of the biological molecule is at a first level, and wherein the biological signal is either present or absent;

(d) gene expression in a second cell of interest;

(e) gene expression in the second cell of interest, wherein the cell has been exposed to a biological signal relevant to the cellular process;

(f) gene expression in the second cell of interest, which cell contains altered levels, relative to physiological levels, of the biological molecule, due to the introduction into the second cell of a heterologous nucleic acid directing expression of the polypeptide, wherein the altered level of the biological molecule is at a second level, and wherein the biological signal is either present or absent; and

identifying a genetic element whose expression differs.

The use of two levels of expression of the heterologous nucleic acid permits the comparison of the effects of each level and of the biological signal on each cell type, and the identification of genetic elements whose expression behaves in the same way, or in different ways, between those levels and biological signals tested. More than two levels of expression of the heterologous nucleic acid can be applied in the same manner with each cell type and each biological signal.

Alternatively, the biological molecule in the first cell may be a different biological molecule to that whose levels are altered in the second cell. In this embodiment, the levels of the biological molecule in the first and second cells may be the same or may be different.

This aspect of the invention thus provides a differential expression screening method for identifying a genetic element involved in a cellular process, which method comprises comparing:

(a) gene expression in a first cell of interest;

(b) gene expression in the first cell of interest, wherein the cell has been exposed to a biological signal relevant to the cellular process;

(c) gene expression in the first cell of interest, which cell contains altered levels, relative to physiological levels, of a first biological molecule whose activity is responsive to the biological signal, due to the introduction into the first cell of a heterologous nucleic acid directing expression of a first polypeptide, wherein the biological signal is either present or absent;

(d) gene expression in a second cell of interest;

(e) gene expression in the second cell of interest, wherein the cell has been exposed to a biological signal relevant to the cellular process;

(f) gene expression in the second cell of interest, which cell contains altered levels, relative to physiological levels, of a second biological molecule, due to the introduction into the second cell of a heterologous nucleic acid directing expression of a second polypeptide, wherein the biological signal is either present or absent; and

identifying a genetic element whose expression differs.

The use of two types of heterologous nucleic acid permits the comparison of the effects of type and of the biological signal on each cell type, and the identification of genetic elements whose expression behaves in the same way, or in different ways, between those types and biological signals. More than two types of the heterologous nucleic acid can be applied in the same manner with each cell type and each biological signal tested. This aspect of the invention has enabled the discovery of genes that are differentially regulated by different biological molecules under particular environmental changes. This raises the possibility of tissue and cell-specific therapeutic modulation of cellular responses.

In all the above embodiments, the first and second cells whose gene expression is compared may be different cell types (for example, healthy cells and diseased cells). The use of two or more cell types permits the comparison of the effects of the different biological signals and of the heterologous nucleic acid on those cell types, and the identification of genetic elements whose expression behaves in the same way, or in different ways, between those cell types and biological signals tested. More than two cell types can be assessed in the same manner.

In a preferred embodiment of the invention, the polypeptide is implicated in a disease process. Accordingly, the first cell may be from a normal patient and the second cell from a diseased patient or vice-versa. Alternatively, the first cell is from a diseased patient and the second cell is from the same diseased patient or from a patient with the same disease.

A further aspect of the invention thus provides a differential expression screening method for identifying a gene or gene product involved in a cellular process which method comprises:

(i) comparing gene expression in:

(a) a first cell of interest; and

(b) a second cell of interest;

(ii) comparing gene expression in

(a) the first cell of interest; and

(b) a third cell of interest which cell comprises altered levels, relative to physiological levels, of a candidate gene or gene product, due to the introduction into the third cell of a heterologous nucleic acid directing amplification or expression of the candidate gene or gene product; and

(iii) selecting those candidate genes or gene products which give rise to an alteration in the levels, copy number or expression of a second gene or gene product in the third cell of interest relative to the first cell of interest, which second gene or gene product also has altered levels, copy number or of expression in the second cell of interest relative to the first cell of interest.

Preferably the candidate gene product is a polypeptide or RNA molecule.

In a preferred embodiment of the above aspect of the invention, a differential expression screening method is provided for identifying a gene product involved in a disease process which method comprises:

(i) comparing gene expression in:

(a) a first cell of interest from a normal patient; and

(b) a second cell of interest from a diseased patient;

(ii) comparing gene expression in

(a) the first cell of interest; and

(b) a third cell of interest from a normal patient which cell comprises altered levels, relative to physiological levels, of a candidate gene or gene product, due to the introduction into the third cell of a heterologous nucleic acid directing amplification or expression of the candidate gene or gene product; and

(iii) selecting those candidate genes or gene products which give rise to an alteration in the levels, copy number or expression of a second gene or gene product in the third cell of interest relative to the first cell of interest, which second gene or gene product also has altered levels, copy number or expression in the second cell of interest relative to the first cell of interest.

In a particularly preferred embodiment of this aspect of the invention, the expression of the gene product is preferably regulated by a signal (such as a biological or other environmental signal relevant to the disease process), and the method includes the steps of comparing gene expression in the cell types at two different levels of the signal.

In the embodiments of the invention described above, the comparison of gene expression is carried out by identifying using nucleic acid techniques those mRNA transcripts whose levels are altered between the different cell types of interest.

In the embodiments of the invention that are described above, the comparison of gene expression may be carried out by identifying, using protein analytical techniques, those polypeptides whose levels are altered between the different cell types of interest.

According to a still further aspect of the invention, there is provided a method of increasing the sensitivity of a differential expression screening method in which gene expression of a first and a second cell of interest in response to two different levels of a signal are compared, the method comprising introducing a heterologous nucleic acid into the first cell or the second cell to increase the level of a biological molecule which modulates the response of the cell to the signal.

### BRIEF DESCRIPTION OF THE DRAWINGS

FIG. 1: Northern blots performed to confirm overexpression of HIF-1 [symbol] and EPAS1 using adenoviral gene transfer in transduced macrophages. RNA loading was as follows: Lanes 1,2: Macrophages transduced with the adenovirus AdApt ires-GFP. Lanes 3,4: Macrophages transduced with the adenovirus AdApt HIF-1 [symbol] ires-GFP. Lanes 4,5: Macrophages transduced with the adenovirus AdApt EPAS\tilde{1}ires-GFP. In lanes 1,3,5 the macrophages were maintained in normoxia (20% O2). In lanes 2,4,6 the macrophages were maintained in hypoxia (0.1% O2). Positions of bands from an RNA size ladder are indicated to the right of each blot in kilobases (kb). Hybridisation probes were complimentary to the genes HIF-1□ (A), EPAS1 (B) and 28s ribosomal RNA (C).

FIG. 2: A scatter plot of two representative RNA samples analysed using Research Genetics GeneFilters. RNA from non-transduced macrophages in normoxia (Y-axis) or hypoxia (X-axis) was hybridised to two Research Genetics GeneFilters GF200 arrays. Analysis was output as normalised intensity for each gene on the array, with two values per gene corresponding to the signals from normoxia and hypoxia. These values were plotted as a scatter graph, with each dot representing a gene on the array. Genes expressed at similar levels between the RNA samples are located at the x=y line. In this representation an indication is apparent of the dynamic range of detection.

FIG. 3: Analysis of Lactate Dehydrogenase A expression with Smartomics. In section A, thumbnail images of spots corresponding to the lactate dehydrogenase-A (LDH-A) gene are shown. Contrast levels were set at a level to allow optimal visualisation of this gene, but are at a constant setting throughout this figure. Each strip of 6 images corresponds to a discrete array position or experiment, over the range of RNA samples. Figures beneath individual spot images are ratios of the normalised intensity of that spot compared to the reference condition (gfp; 20% O2). Array location: Identity of the spot as defined by Research Genetics. Clone: IMAGE identification. The histogram (section B) shows the average of the figures shown and error bars are standard deviation. gfp: cells transduced with AdApt ires-GFP. Hif-1a: Cells transduced with AdApt Hif-1□-ires-GFP. Epas1: Cells transduced with AdApt Epas1-ires-GFP.

FIG. 4: Analysis of Glyceraldehyde 3-phosphate dehydrogenase expression with Smartomics. In section A, thumbnail images of spots corresponding to the glyceraldehyde 3-phosphate dehydrogenase (GAPDH) gene are shown. Contrast levels were set at a level to allow optimal visualisation of this gene, but are at a constant setting throughout this figure. Each strip of 6 images corresponds to a discrete array position or experiment, over the range of RNA samples. Figures beneath individual spot images are ratios of the normalised intensity of that spot compared to the reference condition (gfp; 20% O2). Array location: Identity of the spot as defined by Research Genetics. Clone: IMAGE identification. The histogram (section B) shows the average of the figures shown and error bars are standard deviation. gfp: cells transduced with AdApt ires-GFP. Hif-1a: Cells transduced with AdApt Hif-1□-ires-GFP. Epas1: Cells transduced with AdApt Epas1-ires-GFP.

FIG. 5: Analysis of Platelet derived growth factor beta expression with Smartomics. In section A, thumbnail images of spots corresponding to the Platelet derived growth factor beta (PDGF Beta) gene are shown. Contrast levels were set at a level to allow optimal visualisation of this gene, but are at a constant setting throughout this figure. Each strip of 6 images corresponds to a discrete array position or experiment, over the range of RNA samples. Figures beneath individual spot images are ratios of the normalised intensity of that spot compared to the reference condition (gfp; 20% O2). Array location: Identity of the spot as defined by Research Genetics. Clone: IMAGE identification. For this gene, different IMAGE clones corresponding to the same gene are present. The histogram (section B) shows the average of the figures shown and error bars are standard deviation. gfp: cells transduced with AdApt ires-GFP. Hif-1a: Cells transduced with AdApt Hif-1□-ires-GFP. Epas1: Cells transduced with AdApt Epas1-ires-GFP.

FIG. 6: Analysis of Monocyte Chemotactic Protein-1 expression with Smartomics. In section A, thumbnail images of spots corresponding to the Monocyte Chemotactic Protein-1 (MCP-1) gene are shown. Contrast levels were set at a level to allow optimal visualisation of this gene, but are at a constant setting throughout this figure. Each strip of 6 images corresponds to a separate experiment, over the range of RNA samples. Figures beneath individual spot images are ratios of the normalised intensity of that spot compared to the reference condition (gfp; 20% O2). Array location: Identity of the spot as defined by Research Genetics. Clone: IMAGE identification. The histogram (section B) shows the average of the figures shown and error bars are standard deviation. gfp: cells transduced with AdApt ires-GFP. Hif-1a: Cells transduced with AdApt Hif-1□-ires-GFP. Epas1: Cells transduced with AdApt Epas1-ires-GFP.

FIG. 7: Discovery of a novel gene (Hs.16335) using Smartomics. In section A, thumbnail images of spots corresponding to the EST from UniGene cluster Hs.16335 are shown. Contrast levels were set at a level to allow optimal visualisation of this gene, but are at a constant setting throughout this figure. For this gene, contrast levels are at maximum. Each strip of 6 images corresponds to a separate experiment, over the range of RNA samples. Figures beneath individual spot images are ratios of the normalised intensity of that spot compared to the reference condition (gfp; 20% O2). Array location: Identity of the spot as defined by Research Genetics. Clone: IMAGE identification. The histogram (section B) shows the average of the figures shown and error bars are standard deviation. gfp: cells transduced with AdApt ires-GFP. Hif-1a: Cells transduced with AdApt Hif-1□-ires-GFP. Epas1: Cells transduced with AdApt Epas1-ires-GFP.

FIG. 8: Virtual Northern blot hybridisation to validate discovery of Hs.16335 by Smartomics. A) Hybridisation probe=Hs.16335. B) Hybridisation probe=β actin. Lanes 1-6 are the RNA samples used in FIGS. 3-7, from cells transduced with adenovirus. Lanes 7-10 are from non-transduced macrophages with (lanes 9,10) or without (lanes 7,8) prior activation. Histograms show relative mRNA expression levels, from phosphorimager analysis, relating to the Northern blots positioned above. Figures are relative expression ratios compared to gfp (20% O2).

FIG. 9: Plasmid map for pONY8Z.

FIG. 10: Plasmid map for pONY8.1SM.

FIG. 11: Plasmid map for pSMART CMV-HIF.

FIG. 12: Plasmid map for pSMART CMV-empty.

### DETAILED DESCRIPTION OF THE INVENTION

Although in general the techniques mentioned herein are well known in the art, reference maybe made in particular to Sambrook et al., Molecular Cloning, A Laboratory Manual (1989) and Ausubel et al., Short Protocols in Molecular Biology (1999) 4^{th} Ed, John Wiley & Sons, Inc.

Unless defined otherwise, all technical and scientific terms used herein have the same meaning as commonly understood by one of ordinary skill in the art to which this invention belongs.

### A. Differential Expression Screening Techniques

Genes encode gene products, mainly polypeptides but also RNAs, that are involved in a huge variety of cellular processes. The technique of differential expression screening is based on the idea that by comparing expression under two sets of conditions, genes whose expression varies between those two conditions can be identified and their function related back to the differences between those conditions. For example, genes involved in a pathway responsive to mitogens such as platelet-derived growth factor (PDGF) can be identified by comparing gene expression in cells exposed to PDGF versus gene expression in cells not exposed to PDGF.

Thus the term “differential expression screening” as used herein means comparing gene expression between two cells under different conditions or two different cells under the same or different conditions, with the aim of identifying genes or gene products that differ in their levels of expression between the two cells.

The differences in gene expression may be measured using a variety of techniques. The first main type of technique is based on the measurement of nucleic acids and is termed herein as “genomic or cDNA techniques”. A useful review is provided in Kozian and Kirschbaum (1999). The second main type of technique is based on the measurement of cellular protein content and is termed herein as “proteomic techniques”.

Genomic or cDNA Techniques

One method well known in the art is subtractive cDNA hybridisation. This technique involves hybridising a population of mRNAs from one cell (e.g. a control cell) with a population of cDNAs made from the mRNA of another cell (e.g. a cell exposed to PDGF). This step will remove all sequences from the cDNA preparation that are common to both cells. The cDNAs derived from mRNAs whose expression is upregulated in the cell exposed to PDGF will not have a corresponding mRNA from the control with which to hybridise and can be isolated. Typically, the cDNAs are also hybridised with mRNA from the same cell to confirm that they represent coding sequences. This procedure is described in detail in WO90/11361 where mRNA from cells from the roots of plants treated with a chemical, N-(amincarbonyl)-2-chlorobenzenesulphonamide, were used to produce a cDNA library that was then hybridised with mRNA from untreated root cells. The procedure identified a number of genes whose expression was upregulated by the chemical.

The polymerase chain reaction (PCR) has led to the development of a number of other methods. RT-PCR differential display was first described by Liang and Pardee (1992). This technique involves the use of oligo-dT primers and random oligonucleotide 10-mers to carry out PCR on reverse-transcribed RNA from different cell populations. PCR is often carried out using a radiolabelled nucleotide so that the products can be visualised after gel electrophoresis and autoradiography. Wilkinson et al. (1995) used PCR differential display to identify five mRNAs that are upregulated in strawberry fruit during ripening. A review of differential display RT-PCR (also known as differential display of mRNA) is provided in Zhang et al. (1998) and a recent improvement using ‘long distance’ PCR is described in Zhao et al. (1999).

Another technique is termed cDNA library screening. A review of this technique and the other two differential expression screening techniques mentioned above is provided in Maser and Calvet (1995).

Differential display competitive PCR is a fairly recent innovation that has been successfully used to study changes in global gene expression in situations where only a few genes change expression levels, such as exposure of MCF17 cell to oestradiol, and in more complex situations such as neuronal differentiation of human NTERA2 cells (Jorgensen et al., 1999).

Other techniques that are suitable for the analysis of the transcriptome of a specific cell type include serial analysis of gene expression (SAGE; Velculescu et al., Science (1995) 270; 484-487), Selective amplification via biotin- and restriction-mediated enrichment (SABRE) (Lavery et al, (1997), PNAS USA 94: p6831-6836); Differential display (for example, indexing differential display reverse transcriptase polymerase chain reaction (DDRT-PCR; Mahadeva et al. (1998) J. Mol.Biol. 284, 1391-1398)); representational difference analysis (RDA) (Hubank (1999) Methods in Enzymology 303: 325-349; see Kozian and Kirschbaum (1999) for review and references therein); differential screening of cDNA libraries (see Sagerstrom et al. (1997) Annu. Rev. Biochem. 66: 751-783); “Advanced Molecular Biology”, R. M. Twyman (1998) Bios Scientific Publishers, Oxford; “Nucleic Acid Hybridization”, M. L. M. Anderson (1999) Bios Scientific Publishers, Oxford); Northern blotting; RNAse protection assays; S1-nuclease protection assays; RT-PCR; real time RT-PCR (Taq-man); EST sequencing; massively parallel signature sequencing (MPSS); and sequencing by hybridisation (SBH) (see Drmanac R. et al (1999), Methods in Enzymology 303:165-178). Many of these techniques are reviewed in “Comparative gene-expression analysis” Trends Biotechnol. 1999 February;17(2):73-8.

The actual identification of gene products whose expression differs between the two cell populations can be carried out in a number of ways. Subtractive methods will inherently identify gene products whose expression differs since gene products whose expression is the same are eliminated from the sample. Other methods include simply comparing the expression products from one cell with the expression products from another and looking for any differences (with PCR-based techniques, the number of products in each sample can be limited to a reasonable size), optionally with the aid of a computer program. For example using a PCR-based technique a visual comparison of bands present in different lanes allows the identification of bands unique to one lane. These bands can be cut out of the gel and subsequently analysed.

The advent of DNA chip technology, allows comparisons to be conveniently conducted by the use of microarrays (see Kozian and Kirschbaum, 1999 for review and references therein). Typically, arrays are generated using cDNAs (including ESTs), PCR products, cloned DNA and synthetic oligonucleotides that are fixed to a substrate such as nylon filters, glass slides or silicon chips. To determine differences in gene expression, labelled cDNAs or PCR products are hybridised to the array and the hybridisation patterns compared. The use of fluorescently labelled probes allows mRNA from two different cell populations to be analysed simultaneously on one chip and the results measured at different wavelengths. A microarray-based differential expression screening technique is described in U.S. Pat. No. 5,800,992.

Proteomic Techniques

Proteomics is the study of proteins' properties on a large scale to obtain a global, integrated view of disease processes, cellular processes and networks at the protein level. A review of techniques used in proteomics is given in Blackstock and Weir (1999)—see also references provided therein. The methods of the present invention are mainly concerned with expression proteomics, the study of global changes in protein expression in cells using electrophoretic techniques and image analysis to resolve proteins. Whereas nucleic acid analysis emphasises the message, proteomics is more concerned with the product. The two approaches are sometimes complementary since proteomic techniques may be useful in detecting changes in polypeptide levels that are due to changes in protein stability rather than mRNA levels.

A well known and ubiquitous technique used in the field of proteomics involves measuring the polypeptide content of a cell using 2D polyacrylamide gel electrophoresis (PAGE) and comparing this with the polypeptide content of another cell. The results of electrophoresis are typically a gel visualised with a dye such as silver stain or Coomassie-blue, or an autoradiograph produced from the gel, all with spots corresponding to individual proteins. Fluorescent dyes are also available.

The aim is therefore to identify spots that differ between the two gels/autoradiographs, i.e. missing from one, reduced in intensity or increased in intensity. Thus in the case of proteomics, comparing gene expression simply involves comparing the protein profile from one cell with the protein profile from another. Commercial software packages are available for automated spot detection.

Spots of interest may be excised from gels and the proteins identified using techniques such as matrix-assisted-laser-desorption-ionisation-time-of-flight (MALDI-TOF) mass spectrometry and electrospray mass spectrometry (see “Proteomics to study genes and genomes” Akhilesh Pandey and Matthias Mann, (2000), Nature 405: 837-846).

It may be desirable to perform some measure of prefractionation, such as centrifugation or free-flow electrophoresis to improve the identification of low abundance proteins. Special procedures have also been developed for basic proteins, membrane proteins and other poorly soluble proteins (Rabilloud et al., 1997).

Additionally, the recent developments in the field of protein and antibody arrays now allow the simultaneous detection of a large number of proteins. For example, low-density protein arrays on filter membranes, such as the universal protein array system (Ge H, (2000) Nucleic Acids Res. 28(2), e3) allow imaging of arrayed antigens using standard ELISA techniques and a scanning charge-coupled device (CCD) detector. Immuno-sensor arrays have also been developed that enable the simultaneous detection of clinical analytes. It is now possible using protein arrays, to profile protein expression in bodily fluids, such as in sera of healthy or diseased subjects, as well as in patients pre- and post-drug treatment.

Antibody arrays also facilitate the extensive parallel analysis of numerous proteins that are hypothetically implicated in a disease or particular physiological state. A number of methods for the preparation of antibody arrays have recently been reported (see Cahill, Trends in Biotechnology, 2000 7:47-51).

The above discussion provides a description of prior art methods available to the skilled person for performing differential expression screening of two or more cell populations in a general sense. The introduction of heterologous genes for the purpose of examining changes in general gene expression has also been described (Busch and Bishop, J Immunol, 1999 162:2555-2561; Robinson et al, Proc Natl Acad Sci USA, 1997 94:7170-7175). However, the present invention is distinguished from these prior art methods in that a further step is required, namely that the levels of particular endogenous biological molecules in a cell are altered by the experimenter, so that the levels of gene products that are responsive to cellular perturbations such as signalling events and are affected by the biological molecule(s) become more readily detectable. In other words, the object is to amplify and/or increase the signal to noise ratio of the differential response normally obtained so as to increase the likelihood of detecting gene products whose levels in a cell are low and/or whose expression normally changes by only a small amount.

By way of an example, the transcription factor HIF-1α is responsive to intracellular oxygen levels. Decreases in oxygen levels increase HIF-1α activity and lead to increased transcription from genes controlled by a hypoxia responsive element (HRE). If the levels of HIF-1α in the cell are raised artificially, for example by infecting cells with a viral vector that directs expression of HIF-1α, then an increase in the transcriptional response mediated by HIF-1α is expected. Consequently, changes in the expression of genes whose expression is sensitive to the hypoxia, and mediated by HIF-1α induction, should be greater than in normal cells expressing physiological levels of HIF-1α.

### B. Biological Molecules

The biological molecule can be any compound that is found in cells as a result of anabolic or catabolic processes within a cell or as a result of uptake from the extracellular environment, by whatever means. The term “biological molecule” means that the molecule has activity in a biological sense. Preferably the biological molecule is synthesised within the cell, i.e. is endogenous to that cell, or in the case of multicellular organisms, also within any of the cells of the organism.

Examples of biological molecules will therefore include proteins, peptides, nucleic acids, carbohydrates, lipids, steroids, co-factors, mimetics, prosthetic groups (such as haem), inorganic molecules, ions (such as Ca²⁺), inositides, hormones, growth factors, cytokines, chemokines, inflammatory agents, toxins, metabolites, pharmaceutical agents, plasma-borne nutrients (including glucose, amino acids, co-factors, mineral salts, proteins and lipids), foreign or pathological extracellular components, intracellular and extracellular pathogens (including bacteria, viruses, fungi and mycoplasma). Where appropriate, precursors, monomeric, oligomeric and polymeric forms, and breakdown products of the above are also included.

Examples of polypeptide biological molecules include enzymes, transcription factors, hormones, structural components of cells and receptors, including membrane bound receptors.

Preferably, the biological molecule is known to be involved in the cellular process of interest.

In one embodiment of the invention, the biological molecule is responsive to a change in condition of the cellular environment, also referred to herein as a signal. Examples of such environmental conditions or signals include changes in the cellular microenvironment, exposure to hormones, growth factors, cytokines, chemokines, inflammatory agents, toxins, metabolites, pH, pharmaceutical agents, hypoxia, anoxia, ischemia, imbalance of any plasma-borne nutrient [including glucose, amino acids, co-factors, mineral salts, proteins and lipids], osmotic stress, temperature [hypo and hyper-thermia], mechanical stress, irradiation [ionising or non-ionising], cell-extracellular matrix interactions, cell-cell interactions, accumulations of foreign or pathological extracellular components, intracellular and extracellular pathogens [including bacteria, viruses, fungi and mycoplasma] and genetic perturbations [both epigenetic or mediated by mutation or polymorphism]. As is clear from the above list, the signal may be an externally applied signal such as an environmental signal, for example redox stress, the binding of an extracellular ligand to a cell surface receptor leading to a cellular response mediated by a signal transduction signal. Alternatively, the signal may be an internally applied signal such as an increase in kinase activity due to falling levels of a cell metabolite.

The levels of the biological molecule may be altered directly or indirectly. Direct alteration may be achieved by, for example, causing cells to take up the molecule by incubating cells in a medium containing levels of the molecule that are altered from physiological levels, for example, higher physiological levels, of the molecule. Other methods include vesicle-mediated delivery and microinjection. In the case of nucleic acids and polypeptides, the level of the biological molecule in the cell may be raised by the introduction of a heterologous nucleic acid into the cell which directs the expression of the nucleic acid or polypeptide.

The term “heterologous nucleic acid” in the present context means that the nucleic acid is not present in its natural context i.e. the cell has been modified so as to contain the nucleic acid which would otherwise not be present in the form in which it is introduced. For example, the nucleic acid may be extrachromosomal, such as encoded on a bacterial plasmid, bacteriophage, transposon, yeast episome, insertion element, yeast chromosomal element, a virus (including, for example, baculoviruses and SV40 (simian virus), vaccinia viruses, adenoviruses, fowl pox viruses, pseudorabies viruses and retroviruses, or combinations thereof, such as those derived from plasmid and bacteriophage genetic elements, including cosmids and phagemids. The nucleic acid may be incorporated into the chromosome, such as by the use of retroviral vectors, including murine or feline leukaemia virus, or the Lentiviruses human immunodeficiency virus and equine infectious anaemia virus. Human, bacterial and yeast artificial chromosomes (HACs, BACs and YACs respectively) may also be employed to deliver larger fragments of DNA than can be contained and expressed in other vectors. The nucleic acid may also be integrated into the genome, for example, by viral transduction or by homologous recombination (see, for example, International patent application WO99/29837), or by the microinjection techniques used to generate transgenic animal embryos or stem cells. Nonetheless, part or all of the heterologous nucleic acid molecule may be identical to a corresponding genomic sequence, since the introduction of additional copies of a gene is a convenient means for increasing the levels of expression of that gene.

Indirect means for altering the levels of the biological molecule are numerous and include increasing the levels of an inhibitory or stimulatory molecule using the methods described above. Inhibitory molecules include antisense nucleic acids, ribozyme or an EGS (external guide sequence) directed against the mRNA encoding the biological molecule, a transdominant negative mutant directed against the biological molecule, transcription factors, enzyme inhibitors, and intracellular antibodies, such as scFvs. Examples of stimulatory molecules include enzyme activators, and transcriptional activators. Thus, cells may be manipulated in a number of ways such that ultimately the levels of the biological molecule are altered. Reduced expression may be achieved by expressing an anti-sense RNA.

According to the invention, the levels of the biological molecule should be altered relative to physiological levels. Thus they may be enhanced or reduced. The term “relative to physiological levels” means relative to the concentration or activity of the biological molecule typically present in the cell type under normal physiological conditions prior to manipulation of those levels. Thus the intention is that by deliberate means, the activity of the biological molecule is altered above or below that which is found in the cell under a range of normal physiological conditions. “Physiological conditions” includes the conditions normally found in vivo and the conditions normally used in vitro to culture the cells.

By way of an example, the activity or concentration may be increased or decreased 2-fold, 5-fold, 10-fold, 20-fold, 50-fold or 100-fold compared to the normal physiological activity or concentration found in the cell prior to introducing, for example, the heterologous nucleic acid.

The invention allows the identification of genetic elements that are involved in a cellular process. As discussed above, the term “genetic element” is meant to include genes, gene products (such as RNA molecules, and polypeptides), and cis-acting regulatory elements (such as promoter elements and enhancer elements). Compared to conventional differential screening techniques, the invention considerably facilitates the identification of genes and gene products that are involved in a cellular process, since the level and/or ratio of signal to noise is considerably improved using the described method.

Of particular note is the ability that the invention imparts to identify genes and gene products involved in a cellular process, and thus to investigate the role of these genes and gene products further. For example, if a particular polypeptide is known to have a role in a cellular process, this paves the way for the development of agents that modify or regulate the polypeptide, and thus influence the cellular process itself. Such information clearly has great relevance in the analysis, diagnosis and treatment of disease, in identifying candidate points for intervention, and paving the way for the development of agents that are able to prevent or redress any physiological imbalance in any cellular process that leads to undesirable effects, such as disease.

In addition to identifying genes and gene products, the invention allows the identification of other elements that are associated with genes that are implicated in a particular cellular process. Examples of such elements include promoter elements and enhancer elements that regulate the transcription of genes that are expressed in the cellular process. The identification of such elements would have great value in the study of cellular processes, and, for example, would pave the way for the development of synthetic regulatory elements that are responsive to biological signals generated in a particular cellular process.

Included in this aspect of the invention is the identification of mutations and polymorphisms in genes and their regulatory elements, that affect the response of the gene to the cellular process under study. This type of information would be of great value in evaluating and dissecting the differences in expression patterns that are found between different individuals under different biological conditions.

The differential expression screening method of the invention also allows the molecular dissection of biological pathways, by altering particular aspects of the pathway under study, as desired. In this way, the method of the invention is advantageous over conventional differential expression screening methods that are known in the art. These prior art methods compare gene expression profiles between cell populations under different biological conditions, and thus generate a global perspective on the gene expression patterns in the two populations, even if heterologous nucleic acids are used without reference to specific biological pathways and responses. In contrast, by influencing the level of a particular biological molecule that is implicated in the pathway under study, through the introduction of a heterologous nucleic acid into one cell population, the method of the invention allows a pathway to be dissected into its precise molecular components.

This aspect of the invention may be illustrated with the particular example of the biological response to hypoxia, although the skilled reader will appreciate that analogous cellular processes will be equally applicable to study by this method. The biological response to hypoxia is complex, having a large number of participating molecular components. Two important components are the proteins HIF1α and EPAS1. By introducing into one cell population, a heterologous nucleic acid encoding HIF1α, this allows the evaluation of the differences in gene expression profile that are generated by HIF1α itself. A similar experiment, performed using a heterologous nucleic acid encoding EPAS1, allows the dissection of this particular aspect of the molecular response to hypoxia. By identifying molecular components that are regulated by one pathway (HIF1α) and not the other (EPAS1), this cellular process can be selectively regulated, for example, using agents that are specific to a component of the HIF1α pathway. The application of the present invention to the hypoxic response has enabled the discovery of novel genes which are differentially regulated by HIF1α and EPAS1, and thus has raised the possibility of tissue and cell-specific therapeutic modulation of the cellular response to hypoxia.

HIF1α agonists or antagonists potentially have application to up or down-regulate, respectively, responses to hypoxia such as angiogenesis and erythropoiesis. For example, it is known that the production of erythropoietin in the kidney is regulated by HIF1α (Bunn et al (1998) Erythropoietin: a model system for studying oxygen-dependent regulation, J Exp Biol 201:1197-1201), and thus HIF1α antagonists may cause anaemia by down-regulation of erythropoietin. The application of the present invention to the identification of genes which are differentially regulated by HIF1α and EPAS1, and the clear recognition of the different effects of these two closely-related transcription factors, permits the development of EPAS1 agonists or antagonists, or modulators of the activity of specific differentially-regulated genes, to overcome any potentially negative clinical effects of HIF1α modulation, and thereby enable the identification and development of diagnostic and therapeutic products for diagnosing and treating hypoxia-related diseases.

Whereas in a preferred embodiment of the invention, the levels of the biological molecule are altered by the introduction of a heterologous nucleic acid, typically a nucleic acid that directs expression of a polypeptide, the heterologous nucleic acid should comprise a coding sequence operably linked to a control sequence that is capable of providing for the expression of the coding sequence by the host cell, i.e. the vector is an expression vector. The term “operably linked” means that the components described are in a relationship permitting them to function in their intended manner. A regulatory sequence “operably linked” to a coding sequence may be ligated to the coding sequence in such a way that expression of the coding sequence is achieved under conditions compatible with the control sequences.

The control sequences may be modified, for example, by the addition of further transcriptional regulatory elements to make the level of transcription directed by the control sequences more responsive to transcriptional modulators.

Control sequences suitable to be operably linked to sequences encoding the protein of the invention include promoters/enhancers and other expression regulation signals. These control sequences may be selected to be compatible with the host cell in which the expression vector is designed to be used. The term “promoter” is well known in the art and encompasses nucleic acid regions ranging in size and complexity from minimal promoters to promoters including upstream elements and enhancers.

The promoter is typically selected from promoters that are functional in mammalian cells, although promoters functional in prokaryotic cells or other eukaryotic cells may be used where appropriate. Thus, the promoter is typically derived from promoter sequences of viral or eukaryotic genes. For example, it may be a promoter derived from the genome of a cell in which expression is to occur. Eukaryotic promoters may be promoters that function in a ubiquitous manner (such as promoters of α-actin, β-actin, tubulin) or, alternatively, a tissue-specific manner (such as promoters of the genes for pyruvate kinase). Tissue-specific promoters specific for particular cells may be used. They may also be promoters that respond to specific stimuli, for example promoters that bind steroid hormone receptors. Viral promoters may also be used, for example the Moloney murine leukaemia virus long terminal repeat (MMLV LTR) promoter, the Rous sarcoma virus (RSV) LTR promoter or the human cytomegalovirus (CMV) IE promoter.

It may be advantageous for the promoters to be inducible so that the levels of expression from the heterologous nucleic acid can be regulated during the lifetime of the cell. Inducible means that the levels of expression obtained using the promoter can be regulated.

In addition, any of these promoters may be modified by the addition of further regulatory sequences, for example enhancer sequences. Chimeric promoters may also be used comprising sequence elements from two or more different promoters described above.

Examples of suitable vectors include plasmids, artificial chromosomes and viral vectors. Viral vectors include adenoviral vectors, herpes simplex viral vectors, and retroviral vectors. Vectors/polynucleotides may be introduced into suitable host cells using a variety of techniques known in the art, such as transfection, transformation, electroporation, infection with recombinant viral vectors such as retroviruses, herpes simplex viruses and adenoviruses, direct injection of nucleic acids and biolistic transformation. It is particularly preferred to use recombinant viral vector-mediated techniques.

Viral Vectors

The viral vectors used to introduce heterologous nucleic acids into cells according to the present invention may be derived from or may be derivable from any suitable virus. A large number of different viruses have been identified, and subclasses exist, including retroviruses, lentiviruses, which are a subclass of retroviruses, adenoviruses and herpes simplex virus. Examples of retroviruses include: murine leukemia virus (MLV), human immunodeficiency virus, type 1 (HIV-1), human immunodeficiency virus, type 2 (HIV-2), simian immunodeficiency virus, human T-cell leukaemia virus (HTLV), equine infectious anaemia virus (EIAV), feline immunodeficiency virus (FIV), bovine immunodeficiency virus (BIV), Jembrana virus, simian immunodeficiency virus (SIV), caprine arthritis-encephalitis virus (CAEV), gibbon ape leukemia virus (GALV), spleen focus forming virus (SFFV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV). A detailed list of retroviruses may be found in Coffin et al., 1997, “Retroviruses”, Cold Spring Harbour Laboratory Press Eds: J M Coffin, S M Hughes, H E Varmus pp 758-763.

Details on the genomic structure of many retroviruses may be found in the art. By way of example, details on HIV, EIAV and Mo-MLV may be found from the NCBI Genbank (Genome Accession Nos. AF033819, U01866 and AF033811, respectively).

The lentivirus subgroup of retroviruses can be split even further into “primate” and “non-primate” viruses. Examples of primate lentiviruses include the human immunodeficiency virus, type 1 (HIV-1), the causative agent of acquired-immunodeficiency syndrome (AIDS), and simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV),bovine immunodeficiency virus (BIV) and Jembrana virus.

The basic structure of a retrovirus genome is a 5′ LTR and a 3′ LTR, between or within which are located a packaging signal (psi) to enable the genome to be packaged, a primer binding site, integration sites to enable integration into a host cell genome and gag, pol and env genes encoding the packaging components—these are polypeptides required for the assembly of viral particles. More complex retroviruses have additional features, such as rev and RRE sequences in HIV, which enable the efficient export of RNA transcripts of the integrated provirus from the nucleus to the cytoplasm of an infected target cell. Additional features present in the HIV-1 genome are tat, vif, vpu vpr, and nef which encode accessory proteins which are essential for infectivity of the virus or modulate the infectivity of the virus. An additional feature present in the genomes of lentiviruses is the central polypurine tract/central termination sequence (cPPT/CTS) which facilitates infection of non-dividing cells.

In the provirus, these genes and other elements are flanked at both ends by regions called long terminal repeats (LTRs). The LTRs are responsible for proviral integration, and transcription. As such they contain enhancer-promoter sequences and can control the expression of the viral genes. Encapsidation of the retroviral RNAs occurs by virtue of a psi sequence which is located near the 5′ end of the viral genome.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses. The R regions at both ends of the viral RNA are repeated sequences, whereas U5 and U3 represent unique sequences at the 5′- and 3′-ends of the RNA genome, respectively.

In a typical retroviral vector for use in the screening methods of the invention, at least part of one or more of the gag, pol and env protein coding regions essential for replication of the virus may be removed. This makes the retroviral vector replication-defective. Other modifications, such as the removal of promoter/enhancer elements from the U3 region, or deletion of genes for accessory proteins, can also render the vector replication defective. The removed portions may even be replaced by a nucleotide sequence of interest (NOI), such as a nucleotide sequence encoding a biological molecule as described above, to generate a vector capable of integrating its genome into a host genome but wherein the modified viral genome is unable to propagate itself due to a lack of structural proteins.

When integrated in the host genome, expression of the NOI occurs either as a result of transcription from the LTR of the vector or as a result of transcription from a promoter sequence placed in an appropriate position, for example, between the LTR's, and with respect to the NOI. It should be noted that it also possible to replace the viral promoter present in the LTR with a different promoter. The promoter sequence will typically be active in mammalian cells. The promoter sequence driving expression of the one or more first nucleotide sequences may be, for example, a constitutive or a regulated. The promoter may, for example, be a viral promoter such as the natural viral promoter or a CMV promoter or it may be a mammalian promoter. It is particularly preferred to use a promoter that is preferentially active in a particular cell type or tissue type or that can be regulated. Thus, in one embodiment, a tissue-specific regulatory sequence may be used. In mammalian cells an example of a regulatable promoter system is the tetracycline-inducible promoter system (Clontech, Palo Alto, Calif.).

Thus, the transfer of an NOI into a site of interest is typically achieved by: integrating the NOI into the recombinant viral vector; packaging the modified viral vector into a virion particle; and allowing transduction of a site of interest—such as a targeted cell or a targeted cell population.

A minimal genome of a retroviral vector for use in the present invention will therefore comprise (5′) R-U5—a packaging signal (psi) and one or more first nucleotide sequences—U3-R (3′). However, the plasmid vector used to produce the vector genome within a host cell/packaging cell will also include transcriptional regulatory control sequences operably linked to the vector genome to direct transcription of the genome in a host cell/packaging cell. These regulatory sequences may be the natural sequences associated with the transcribed retroviral sequence, i.e. the 5′ U3 region, or they may be a heterologous promoter such as another viral promoter, for example, the CMV promoter.

Production of Retroviral Vectors

Replication-defective retroviral vectors can be produced by using either producer cell lines, packaging cell lines or by transient transfection of a suitable cell line.

Producer cell lines are cell lines which express all the components required for assembly of vector particles capable of transduction. That is, they express gag/pol and envelope proteins, which are required for formation of vector particles and produce transcripts of the vector genome which are packaged into vector particles. Conventionally, producer cells differ from packaging cells only by the fact that they also stably express the vector RNA. The vector RNA can be introduced into the packaging cell, to make the producer cell, either by transfection of a plasmid which is capable of directing expression of the vector RNA, or by transduction of a vector genome which is capable of directing synthesis of vector RNA following integration into the nuclear DNA of the host cell. Packaging cells can also be converted into producer cells on a temporary basis by transient transfection of a plasmid which directs the transcription of vector RNA. A producer cell can also be made from a cell line which comprises only two of the three components required for formation of transduction competent vector particles. For example, in the field of MLV vectors, the TelCEB cell line stably expresses MLV gag/pol and the genome of the MLV vector, MFGnlsLacZ. It can be converted to a producer cell line by introduction of a plasmid which directs expression of an envelope gene. In this respect it should be noted that while the gag/pol genes are derived from the same virus, the env may be derived from the same virus or be from a different virus. When infectious particles are formed as a result of the use of an envelope function from a different virus, the vector particles are said to have been ‘pseudotyped. For example, in the field of lentiviral vectors, it is common to make vectors which are pseudotyped by the G protein of the rhabdovirus, vesicular stomatitis virus.

Vector particles can also made transiently, by transfection of a suitable cell line with plasmids which express the components required for transduction particle formation. For example, MLV, EIAV or HIV vector particles can be produced by transfection of the human cell line, HEK 293T, with plasmids which direct expression of the gag/pol, vector genome and the envelope (Soneoka et al., 1995). Additional plasmids may also be co-transfected, for example, the purpose of increasing titre.

The transient transfection method may advantageously be used to measure levels of vector production when vectors are being developed. In this regard, transient transfection avoids the longer time required to generate stable vector-producing cell lines and may also be used if the vector or retroviral packaging components are toxic to cells. Components typically used to generate retroviral vectors include a plasmid encoding the gag/pol proteins, a plasmid encoding the env protein and a plasmid containing an NOI. Vector production involves transient transfection of one or more of these components into cells containing the other required components. If the vector encodes toxic genes or genes that interfere with the replication of the host cell, such as inhibitors of the cell cycle or genes that induce apoptosis, it may be difficult to generate stable vector-producing cell lines, but transient transfection can be used to produce the vector before the cells die. Also, cell lines have been developed using transient transfection that produce vector titre levels that are comparable to the levels obtained from stable vector-producing cell lines.

It has now become standard practice within the field of retroviral vectors to arrange for the genes which encode the components for particle formation to be encoded separately. For example, the FLYA13 MLV packaging cell line, has separate transcriptional units for expression of MLV gag/pol and env. This strategy reduces the potential for production of a replication-competent virus since three recombinant events are required for wild type viral production. As recombination is greatly facilitated by homology, reducing or eliminating homology between the genomes of the vector and the helper can also be used to reduce the problem of replication-competent helper virus production.

Producer cells/packaging, cells can be of any suitable cell type. Most commonly, mammalian producer cells are used but other cells, such as insect cells are not excluded. Clearly, the producer cells will need to be capable of efficiently translating the env and gag, pol mRNA. Many suitable producer/packaging cell lines are known in the art. The skilled person is also capable of making suitable packaging cell lines by, for example stably introducing a nucleotide construct encoding a packaging component into a cell line.

It is highly desirable to use high-titre virus preparations in both experimental and practical applications. One techniques for increasing viral is to concentrate of viral stocks. This is conveniently achieved by centrifugation, however other methods such as column chromatography can be used.

Vector systems based on lentiviruses are particularly suited for use in this invention. This is because they are capable of infecting dividing or non-dividing cells. Examples of the non-dividing cells in which gene transfer can be achieved include neurons and haematopoietic stem cells. In addition, lentiviral vectors can be configured so that they express only the NOI in the target cell. In effect they are phenotypically silent. Thus, the process of introducing the transgene causes minimal perturbation to the host cell. Vector systems based on HIV-1, EIAV and FIV have been developed and have been developed to a point where they are described as minimal. Minimal vector systems for HIV-1 and EIAV are described in WO 98/17815 and WO 99/32646 and in Kim et al. (1998) J. Virol, 72, 811-816, and Mitrophanous et al.(1996) Gene Therapy, 6, 1808-1818. In these minimal systems the vector component is engineered to express only the NOI in the target cell and furthermore the expression of viral proteins in the cell used for production is reduced to a minimum. For both the HIV-1 and EIAV systems the only lentiviral genes which must be expressed for infectious particle formation are gag/pol and rev. Rev, working in conjunction with the Rev-response element (RRE), is necessary to achieve the levels of Gag/Pol required for high levels particle formation. One way to reduce the requirement for lentiviral proteins even further is to codon optimise gag/pol. This renders expression independent of Rev/RRE. The process of codon-optimisation of the lentiviral gag/pols is described in WO 99/41397, in Kotsopoulou et al., (2000) J.Virol. 74, 4839-4852. The codon optimisation process for EIAV gag/pol is described in UK Patent Application 0009760.0.

More information concerning the codon optimisation process is given here by way of explanation. Cells from various species differ it their usage of particular codons. This codon bias is reflected in a bias in the relative abundance of particular tRNAs in the cell type. By altering the codons in the sequence so that they are tailored to match the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms.

Codon optimisation has a number of other advantages. By virtue of alterations in their sequences, the nucleotide sequences encoding the packaging components of the viral particles required for assembly of viral particles in the producer cells/packaging cells have RNA instability sequences (INS) eliminated from them. At the same time, the sequence coding sequence for the packaging components is retained so that the viral components encoded by the sequences remain the same, or at least sufficiently similar to ensure that the function of the packaging components is not compromised. Codon optimisation also overcomes the Rev/RRE requirement for export, rendering optimised sequences Rev independent. Codon optimisation also reduces homologous recombination between different constructs within the vector system (for example between the regions of overlap in the gag-pol and env open reading frames). The overall effect of codon optimisation is therefore a notable increase in viral titre and improved safety.

In one approach, only codons relating to INS are codon optimised. However, in highly preferred embodiment, the sequences are codon optimised in their entirety, with the exception of the sequence encompassing the frameshift site. The gag/pol gene comprises two overlapping reading frames encoding gag and pol proteins, respectively. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag/pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimised. Retaining this fragment will enable more efficient expression of the gag-pol proteins. For EIAV the beginning of the overlap has been taken to be nt 1262 (where nucleotide 1 is the A of the gag ATG). The end of the overlap is at nt1461 In order to ensure that the frameshift site and the gag-pol overlap are preserved, the wild type sequence has been retained from nt 1156 to 1465.

Derivations from optimal codon usage may be made, for example, in order to accommodate convenient restriction sites, and conservative amino acid changes may be introduced into the gag-pol proteins.

In a highly preferred embodiment, codon optimisation was based on highly expressed mammalian genes. The third and sometimes the second and third base may be changed.

Due to the degenerate nature of the Genetic Code, it will be appreciated that numerous gag/pol sequences can be achieved by a skilled worker. Also there are many retroviral variants described which can be used as a starting point for generating a codon optimised gag/pol sequence. Lentiviral genomes can be quite variable. For example there are many quasi-species of HIV-1 which are still functional. This is also the case for EIAV. These variants may be used to enhance particular parts of the transduction process. Examples of HIV-1 variants may be found at http://hiv-web.lanl.gov. Details of EIAV clones may be found at the NCBI database: http://www.ncbi.nlm.nih.gov.

The strategy for codon optimised gag-pol sequences can be used in relation to any retrovirus. This would apply to all lentiviruses, including EIAV, FIV, BIV, CAEV, Maedi/Visna, SIV, HIV-1 and HIV-2. In addition this method could be used to increase expression of genes from HTLV-1, HTLV-2, HFV, HSRV and human endogenous retroviruses (HERV), MLV and other retroviruses.

The performance of lentiviral vectors may be enhanced in several ways. Most notably there are modifications to the vector genome which improve the efficiency of transduction and the expression level of the NOI. Both of these types of modification may improve the utility of lentiviral vectors for use in the applications described herein. The efficiency of transduction can be improved by incorporation of an element termed the central polypurine tract and the central termination sequence (cPPT/CTS). This element of approximately 200 nt is naturally located near the centre of the viral genome and has been shown to improve transduction by HIV-1-based vectors (Follenzi et al., (2000) Nat Genet. 2000 June;25(2):217-22: Sirven et al., Blood. 2000 Dececember 15;96(13):4103-10. Expression of the NOI may be improved utilising the woodchuck hepatitis virus post-transcriptional regulatory element (WHPRE). It is a 600 bp element that enhances the expression of proteins by increasing the half-life of mRNA through a mechanism involving enhanced polyadenylation. Its beneficial effect has been demonstrated in a number of vectors including HIV-1 based vectors (Zufferey, J Virol. (1999) April;73(4):2886-92; Ramezani et al., Mol Ther. 2000 November;2(5):458-69). This and other methods of use of the element are described in WO 99/14310.

Vectors derived from poxviruses, which include vectors derived from vaccinia, avian pox virus and entomopox viruses, may also be used achieve expression of NOI in a wide range of target cell type. Their use is reviewed in B Moss. 1996 (Poxviridae: The viruses and their replication In Virology Ed B N Fields et al. Chap 83 pp2637-2671 Lippincott-Raven Publishers; PA USA). The use of vectors derived from alphaviruses and poxvirus are reviewed in M W Carroll et al., 2001 (Mammalian expression systems and vaccination; In Genetically Engineered Viruses, pp 107-158 Ed. C Ring & E Blair BIOS Scientific Publishers Ltd Oxford UK). Adeno-associated viral vectors may also be used as gene transfer vectors and their use is reviewed in the following publication: “Adeno-associated viral vectors for gene transfer and gene therapy” (Bueler, H AUTHOR AFFILIATION: Institut fur Molekularbiologie, Universitat Zurich, Switzerland. SOURCE: Biol Chem 1999 June;380(6):613-22).

### C. Cells of Interest

A cell of interest can be any cell, for example a prokaryotic cell, a fungal cell (for example yeast), a plant cell or an animal cell, such as an insect cell or a mammalian cell, including a human cell. In the case of cells from multicellular organism, cells may be primary cells or immortalised cell lines, they may comprise a tissue sample, or they may be part of a living organism. Although cells are frequently referred to in the singular, in general cells will be part of a cell population.

In the methods of the invention, a comparison is required between gene expression in at least two distinct cells. Typically the first of the two or more cells is termed a reference cell. In a preferred embodiment of the invention, the cells to be used in the comparison are substantially identical in all respects. For example, they may both be cells of the same cell line or obtained from the same tissue in an organism. One or both of the cells may then be manipulated so that they comprise altered levels, relative to physiological levels, of the biological molecule as described in section B. In one embodiment, the first cell is unaltered and the second cell is altered. This is particularly preferred, since it should result in an improved signal to noise ratio. However in another embodiment, both cells are altered.

Nonetheless, it is not necessary that the cells used as the starting point of the investigation be substantially identical. For example, in one aspect of the invention, genes involved in disease processes may be investigated using cells from a diseased organism, such as a mammalian patient. These may be compared with cells from a normal organism or similar cells from the same or a different diseased individual. Where cells from a normal organism and a diseased organism are used, generally the normal cells correspond to the first cell of interest and the diseased cells correspond to the second cell of interest. Consequently, at least the diseased cells are modified as described above in section B so that these cells comprise altered levels of the biological molecule.

In another embodiment of the invention, one cell is a cell comprising a mutant gene, whereas the other cell comprises a wild-type version of the same gene.

Another possibility embraced by the present invention is that the cells are from different tissues or from different stages in development or differentiation.

### D. Uses

The present invention provides a number of improved methods for identifying genes by differential expression screening techniques.

In a first aspect, a method is provided for identifying genes involved in a cellular process. Essentially one of the cell types is manipulated so that the levels within that cell of a biological molecule involved in the cellular process are altered. Typically, this may be achieved by the introduction of a heterologous nucleic acid into the cell to direct the expression of a polypeptide. The polypeptide may be the same as the biological molecule or it may modulate the levels of the biological molecule, as described above.

In general, simply modulating the levels of a biological molecule in one of two identical cells and then measuring gene transcription is not the aim of the methods of the present invention since the effect of the biological molecule on gene expression will be measured in the cells, rather than using the change in the levels of the biological molecule to enhance or reduce the response to an event of interest.

However, where the biological molecule is a gene product, such as a polypeptide, that is produced naturally within the cell, altering the levels of the gene product by the introduction of a heterologous nucleic acid may be used simultaneously both to perturb a cellular process and to enhance the response to such a perturbation, so facilitating the identification of gene products that are involved in that cellular process using differential expression techniques. By way of an example, overexpression of HIF-1α amplifies the downstream elements of the hypoxic response, due its enhanced regulatory effect on HIF-1α mediated transcription.

Nonetheless, in the broader aspects of the present invention, two main possibilities arise. The first possibility is that the two cell types are different and have inherently different gene expression patterns. In this situation, alterations in the levels of the biological molecule can be used to enhance those differences. The two cells may be, for example, from different tissues, or from different stages in development or differentiation. The two cells may also be different by virtue of one cell being from diseased tissue and the other cell from normal tissue. Other configurations envisaged are given in section C above.

The second possibility is that the two cell types are the same, but one of the cells is stimulated in some manner and the other cell is not (or one is stimulated to a greater extent than the other). For example, one cell may be incubated in the presence of a growth factor and the other not. In this example, the growth factor is therefore not the biological molecule but is instead a stimulus or signal designed to perturb gene expression in the cell, the effects of which may be amplified by the biological molecule, which in turn is altered in level by the polypeptide expressed from the heterologous nucleic acid.

Thus, in this aspect of the invention, there is provided a method whereby genes whose expression is regulated by a signal or by an environmental change, are identified by subjecting two distinct cell populations to different levels of a signal or environmental condition, whereby either or both cell populations have been manipulated so as to alter the levels of a biological molecule whose activity is responsive to the signal or environmental condition, and identifying gene products whose expression differs. The term “whose activity is responsive to the signal or environmental condition” includes any biological molecule whose concentration in the cell varies in response to the signal or environmental condition, as well as biological molecules whose properties (such as enzymatic activity or affinity for another cellular component) vary in response to the signal or environmental condition.

Thus, returning to the above growth factor example, the cells that are exposed to the growth factor may have been altered to express increased levels of a transcription factor that is involved in the signal transduction cascade that relates to that particular growth factor. Consequently, the effect of the growth factor will be increased downstream of the transcription factor (in either a negative or a positive sense), so facilitating the identification of differentially expressed genes whose expression is regulated by the transcription factor and, ultimately, by the growth factor.

As discussed above, the signal or environmental condition may be either a physical signal, (such as, for example, a change in redox conditions, CO₂ levels, light, osmotic stress, temperature [hypo and hyper-thermia], mechanical stress, irradiation [ionising or non-ionising], exposure to hypoxia, anoxia, ischemia, or chemical (such as a change in the cellular microenvironment, exposure to ligands that bind to receptors on the cell surface and trigger signal transduction pathways, including hormones, cell surface molecules normally attached to other cells, substrates for enzyme reactions that diffuse into or are transported into the cell, growth factors, cytokines, chemokines, inflammatory agents, toxins, metabolites, pH, pharmaceutical agents, imbalance of a plasma-borne nutrient, cell-extracellular matrix interactions, cell-cell interactions, accumulations of foreign or pathological extracellular components, intracellular and extracellular pathogens [including bacteria, viruses, fungi and mycoplasma] and a genetic perturbation.

The first cell maybe subjected to the signal at a first level and the second cell subjected to the signal at a second level. In one example, the first level may simply be the absence of the signal and the second level may be the presence of the signal, or vice-versa. The levels of the signals may be adjusted so as to provide a discernible difference in gene expression. In an alternative embodiment, both the first and second cells may be compared at both the first and second levels of the signal. The presence of the heterologous nucleic acid in the second cell will amplify the differences in gene expression that are caused by the change in signal.

Preferably, the levels of both the signals are at physiologically relevant levels.

In one aspect of the present invention, knowledge already acquired relating to genes that are involved in a disease or other biological process may be used to generate further information about other genes whose expression is altered in a disease or other biological process. In order to do this, one cell is modified so that the levels of the gene product known to be involved in the disease or other biological process are altered, either directly, for example, by the introduction of a heterologous nucleic acid encoding the gene product, or indirectly as described in section B. Gene expression is then measured in both cells and the results compared to identify gene products whose expression varies.

In this aspect of the invention, the two cells may be identical, except in respect of the change in the levels of the gene product that is known to be involved in the disease or other biological process of interest. The two cells may thus both be normal cells of the same type as a cell type in which the disease or other process manifests itself, or they may both be diseased cells. Alternatively, one cell may be normal, and the other diseased. Preferably, the diseased cell is the modified cell if only one of the cells is modified.

In a further aspect of the invention, differential expression screening methods are used to identify genes involved in a disease or other process in a two stage procedure. Firstly, gene expression is compared between a first cell of interest, for example, a cell from a normal patient, and a second cell of interest, for example, a corresponding cell from a diseased patient. As discussed above, the first cell and the second cell will be different in some aspect, such that they exhibit different expression patterns. This may be because the cells are from different tissues or because they are from different individuals (for example, from a normal patient and from a diseased patient). The cells may be of similar origin but have been treated differently in some respect.

Gene products whose expression differs between the first cell and the second cell are then identified. Secondly, a third cell of interest, essentially identical to the first cell is used in a this screening procedure, where a candidate gene is introduced into the third cell so that levels of the genes are altered (typically raised). Gene expression in this cell is compared with gene expression in the first cell and gene products whose expression differs between the first cell and the third cell that comprises altered levels of the candidate gene are identified. If a gene product whose expression is altered in the second cell also has altered gene expression in the third cell, then the candidate gene is selected for further study. Preferably there is a correlation over two or more gene products, preferably at least four or five gene products to minimise false positives.

The invention will now be described with reference to the examples which are illustrative only and non-limiting. In the examples below, the method of the invention as described above is referred to as “Smartomics”.

### EXAMPLES

### Example 1

The Use of Smartomics for Gene Discovery in Macrophages

Macrophages are associated with a variety of disease conditions, including cancer, atherosclerosis and inflammatory diseases such as arthritis. In many of these conditions, the macrophage secretes factors that exacerbate the disease condition. These factors include angiogenic factors, chemotactic agents and inflammatory cytokines. Some of these factors are known, but it is likely that there are other factors that are currently not known and that may be important targets for therapy. In many disease states, macrophages exist in areas of low oxygen (hypoxia) and it is this physiological state that acts as a signal to turn on a number of genes. Given this background, it is reasonable to suggest that important targets for drug development in the fields of cardiovascular disease, cancer and inflammatory disease may be induced in the hypoxia environment.

A simple approach, that would represent the current state of the art, would be to take a population of monocyte/macrophages, divide them in two and place one set in normal oxygen concentrations and the other set in conditions of low oxygen. RNA or protein molecules from the two sets could then be used in appropriate differential analyses. The goal would be to identify proteins or cDNA molecules that are present under conditions of hypoxia but that are not present in those cells that were maintained in normal oxygen concentrations.

If the present invention were to be applied to the identification of hypoxia-induced genes and proteins in macrophages, it would seek to amplify the difference between hypoxia and normoxia in order to increase the signal to noise ratio. This could be achieved by increasing the response to the hypoxia signal by delivering the Hif1α gene to the macrophages in a configuration in which it is over-expressed. Hif1α is part of a regulatory process that responds to low oxygen. Hif1α and other proteins in the hypoxia-induction pathway interact with an enhancer element called the hypoxia response element (HRE) to switch on transcription of hypoxia-induced genes. The HRE, in various guises, is present at a position upstream from many genes that are known to be switched on in conditions of low oxygen. Overexpression of Hif1α leads to massive over-expression of many hypoxia induced genes and so, in a differential screen, it would amplify the levels of hypoxia-specific cDNAs or proteins. This in turn would increase the probability of detecting those molecular species that may be targets for drug development. In this case, therefore, the approach used according to the present invention would be to compare macrophages that are not overexpressing Hif1α in conditions of normal oxygen with those overexpressing Hif1α in conditions of low oxygen.

Hif1α delivery and expression could be achieved in a number of ways.

Here, we describe the construction of an adenoviral vector that constitutively expresses the transcription factor HIF1α. HIF1α cDNA was isolated from Jurkat mRNA using the following PCR primers that harbour NheI and HpaI restriction sites in the 5′ overhangs respectively:

| | |
|---|---|
| Forward primer: | 5′-CGGCTAGC-GACCGATTCACCATGGAG-3′ |
| Reverse primer: | 5′-CGGTTAAC-GCTCAGTTAACTTGATCC-3′ |

The PCR product was digested with NheI and HpaI restriction enzymes and inserted into the NheI-HapI sites in the Introgene AdApt™ transfer vector which contains the human CMV promoter and SV40 polyA sequences. This vector can be linearised using Pmll prior to co-transfection with the right arm of the adenovirus serotype 5 genome into the E1 expressing cell line PerC6 (911 or 293 cells could also be used).

Generation of the AdCMVHIF1α adenovirus using the PerC6 RCA-free system is described at www.introgene.com (Introgene, Leiden, the Netherlands). Methods for efficient adenoviral transduction of primary human macrophages are described in Griffiths et al., 2000.

Gene expression in transduced and untransduced macrophage populations is compared in a number of possible ways as described below to generate read-outs of genes that are expressed under the control of Hif1α. In addition, transduced cells incubated at oxygen concentrations of less than 0.5% are compared with non-transduced cells.

Total RNA samples are prepared for the analysis of differential gene expression. These are labelled either radioactively or fluorescently, and hybridized to arrays of cDNAs on solid supports. Genes which are upregulated by hypoxia and/or expression of individual HIF proteins produce quantitatively stronger hybridization signals. Array strategies may involve either nylon or glass supports, which are reviewed in Bowtell, 1999. Details of methodologies involved in the glass support approach are detailed in Eisen and Brown, 1999. Here, fluorescently labelled probes are used and hybridization is detected using a laser confocal scanner. For the Nylon support approach, standard molecular biology methods of dot blotting and hybridization are involved as detailed in Molecular Cloning: A laboratory manual Sambrook, J et al, Cold Spring Harbor Laboratory Press. Here, RNA samples to be compared are radioactively labelled and hybridization is detected using a phosphorimager.

Arrays can be purchased from Research Genetics, Huntsville, Ala. or would be fabricated in-house using cDNA clones generated by subtraction cloning (PCR-Select method, owned by Clontech Palo Alto, Calif.). Fabrication would involve use of an arraying robot (MicroGrid, BioRobotics Ltd, Cambridge, UK).

### Example 2

The Use of Smartomics for the Identification of Hypoxia-Regulated Genes in Macrophages

The invention has been applied to the identification of hypoxia-induced genes and proteins in macrophages.

Smartomics was utilised to improve the discovery of genes activated or repressed in response to hypoxia in primary human macrophages. As explained in Example 1, this involves augmenting the natural response to hypoxia, by experimentally introducing a key regulator of the hypoxia response, namely hypoxia inducible factor 1α (HIF-1α). Overexpression of HIF-1α was done either in isolation or was done in combination with exposing the cells to hypoxia. This allowed the detection of resulting gene expression changes that would otherwise have not been detectable in response to hypoxia alone.

Although HIF-1α is well known to mediate responses to hypoxia, other transcription factors are also known or suspected to be involved. These include a protein called endothelial PAS domain protein 1 (EPAS1) or HIF-2α, which shares 48% sequence identity with HIF-1α (“Endothelial PAS domain protein 1 (EPAS1), a transcription factor selectively expressed in endothelial cells.” Tian H, McKnight S L, Russell D W. Genes Dev. Jan. 1, 1997; 11(1):72-82.). Evidence suggests that EPAS1 is especially important in mediating the hypoxia-response in certain cell types, and it is clearly detectable in human macrophages, suggesting a role in this cell type (“The macrophage—a novel system to deliver gene therapy to pathological hypoxia.” Gene Ther. 2000 Februray;7(3):255-62. Griffiths L, Binley K, Iqball S, Kan O, Maxwell P, Ratcliffe P, Lewis C, Harris A, Kingsman S, Naylor S.). In the light of this, the current example also utilises overexpression of EPAS1, as an independent means of improving discovery of hypoxia-responsive genes, to overexpression of HIF-1α. It also illustrates an embodiment of the invention, whereby differences in the response to HIF-1α or EPAS1 (or other mediators of the hypoxia response) may be identified, with the goal of identifying therapeutic target molecules more suitable for specific and efficient treatment of disease.

As discussed in Example 1, the introduction of foreign gene sequences (i.e. HIF-1α or EPAS1) to primary macrophages may be achieved by recombinant adenovirus. As discussed in Example 1, a commercially available system was used to produce adenoviral particles involving the adenoviral transfer vector AdApt, the adenoviral genome plasmid AdEasy and the packaging cell line Per-c6 (Introgene, Leiden, The Netherlands). The standard manufacturer's instructions were followed.

Three derivatives of the AdApt transfer vector have been prepared, named AdApt ires-GFP, AdApt HIF-1α-ires-GFP and AdApt EPAS1-ires-GFP. In these vectors, for convenience, AdApt was modified such that inserted genes (i.e. HIF-1α or EPAS1) expressed from the powerful cytomegalovirus (CMV) promoter were linked to the green fluorescent protein (gfp) marker, by virtue of an internal ribosome entry site (ires). Therefore presence of green fluorescence provides a convenient indicator of viral expression of HIF-1α or EPAS1 in transduced mammalian cells.

Standard molecular biology methods were used to construct the derivatives of AdApt, which included reverse transcriptase PCR (RT-PCR), transfer of DNA fragments between plasmids by restriction digestion, agarose gel DNA fragment separation, “end repairing” double stranded DNA fragments with overhanging ends to produce flush blunt ends, and DNA ligation. Subcloning steps were confirmed by DNA sequencing. These techniques are well known in the art, but reference may be made in particular to Sambrook et al., Molecular Cloning, A Laboratory Manual (1989) and Ausubel et al., Short Protocols in Molecular Biology (1999) 4th Ed, John Wiley & Sons, Inc.

Briefly, AdApt ires-GFP was made by inserting the encephalomyocarditis virus EMCV ires followed by the green fluorescent protein gene (GFP), into the end-repaired HpaI restriction site of AdApt, immediately downstream of and in the same orientation as the CMV promoter. Both EMCV ires and gfp sequences are widely used and can be obtained from commonly available plasmids. SEQ ID NO:1 recites the exact nucleotide sequence of the joined ires-GFP which was inserted into the AdApt plasmid.

The plasmid AdApt HIF-1α-ires-GFP was derived from AdApt ires-GFP by inserting the protein coding sequence of human HIF-1α between the CMV promoter and the ires-GFP elements of AdApt ires-GFP. To do this, human HIF-1α cDNA was cloned by RT-PCR from human mRNA, and the sequence was verified by comparison to the published HIF-1α cDNA nucleotide sequence (Genbank accession U22431). The HIF-1α sequence was ligated as an end-repaired fragment into the end-repaired AgeI restriction site of AdApt ires-GFP [this is also the AgeI restriction site of the parental vector AdApt immediately downstream of the CMV promoter]. The exact DNA sequence encoding HIF-1α that was inserted into AdApt ires-GFP is shown in SEQ ID NO: 2.

The plasmid AdApt EPAS1-ires-GFP was derived from AdApt ires-GFP by inserting the protein coding sequence of human EPAS1 between the CMV promoter and the ires-GFP elements of AdApt ires-GFP. To do this, human EPAS1 cDNA was cloned by reverse transcriptase PCR (RT-PCR) from human mRNA, and the sequence was verified by comparison to the published EPAS1 cDNA nucleotide sequence (GenBank accession U81984). The EPAS1 sequence was ligated as an end-repaired fragment into the end-repaired AgeI restriction site of AdApt ires-GFP [this is also the AgeI restriction site of the parental vector AdApt immediately downstream of the CMV promoter]. The exact DNA sequence containing EPAS1 which was inserted into AdApt ires-GFP is shown in SEQ ID NO: 3.

The-adenoviral transfer-vectors AdLpt HIF-1α-ires-GFP and AdApt EPAS1 -ires-GFP, were verified prior to production of adenoviral particles, for their ability to drive expression of functionally active HIF-1α or EPAS1 protein from the CMV promoter in mammalian cells. This was achieved by transient transfection luciferase-reporter assays as described (Boast K, Binley K, Iqball S, Price T, Spearman H. Kingsman S, Kingsman A, Naylor S. Hum Gene Ther. Sep. 1, 1999; 10(13):2197-208. “Characterisation of physiologically regulated vectors for the treatment of ischemic disease.”).

Using the aforementioned Introgene adenoviral system, caesium-banded, pure adenoviral- particles were produced for each of the vectors AdApt ires-GFP, AdApt HIF-1α-ires-GFP and AdApt EPAS1-ires-GFP. Following the Introgene manual, adenoviral preparations were quantitated by spectrophotometry, yielding values of viral particles (VP) per milliliter.

To isolate human macrophage, monocytes were derived from peripheral blood of healthy human donors. 100 ml bags of buffy coat from the Bristol Blood Transfusion Centre (Bristol, UK) were mixed with an equal volume of RPMI1640 medium (Sigma). This was layered on top of 10 ml ficol-paque (Pharmacia) in 50 ml centrifuge tubes and centrifuged for 25 min at 800 ×g. The interphase layer was removed, washed in MACS buffer (phosphate buffered saline pH 7.2, 0.5% bovine serum albumin, 2 mM EDTA) and resuspended at 80 microliter per 10n7 cells. To this, 20 microliter CD14 Microbeads (Miltenyi Biotec) were added, and the tube incubated at 4 degrees for 15 min. Following this, one wash was performed in MACS buffer at 400×g and the cells were resuspended in 3 ml MACS buffer and separated on an LS+MACS Separation Column (Miltenyi Biotec) positioned on a midi-MACS magnet (Miltenyi Biotec). The column was washed with 3×3 ml MACS buffer. The column was removed from the magnet and cells were eluted in 5 ml MACS buffer using a syringe. Cells were washed in culture medium (AIM V (Sigma) supplemented with 2% human AB serum (Sigma), and resuspended at 2×10n5 cells per ml in the same medium and placed in large teflon-coated culture bags (Sud-Laborbedarf GmbH, 82131 Gauting, Germany) and transferred to a tissue culture incubator (37 degrees, 5% CO2) for 7-10 days. During this period, monocytes spontaneously differentiate to macrophages. This is confirmed by examining cell morphology using phase contrast microscopy. Cells are removed from the bags by placing at 4 degrees for 30 min and emptying the contents.

The macrophages were washed and resuspended in DMEM (Gibco, Paisley, UK) supplemented with 4% fetal bovine serum (Sigma). 4×10⁶ cells were plated into individual 10 cm Primeria (Falcon) tissue culture dishes in a total volume of 8 ml per plate, with 6×10⁹ adenoviral particles per ml. Following culture for 16 hr, during which the macrophages adhere to the plate and are infected by the adenoviral particles, the medium is removed and replaced by AIM V medium supplemented with 2% human AB serum. A further 24 hr period of culture is allowed prior to experimentation, to allow gene expression from the transduced adenovirus.

The above dosage of adenoviral particles was determined to be the minimum amount required to achieve transduction of the majority (over 80%) of the macrophage population, using green fluorescence as a marker of gene transfer. This was confirmed using a separate adenoviral construct containing the LacZ reporter gene. By selecting the minimum dose of virus, possible non-specific effects of viral transfer are minimised.

For experimentation with hypoxia, identical culture dishes were divided into two separate incubators: One at 37 degrees, 5% CO2, 95% air (=Normoxia) and the other at 37 degrees, 5% CO2, 94.9% Nitrogen, 0.1% Oxygen (=Hypoxia). After 8 hours culture under these conditions, the dishes were removed from the incubator, placed on a chilled platform, washed in cold PBS and total RNA was extracted using RNazol B (Tel-Test, Inc; distributed by Biogenesis Ltd) following the manufacturer's instructions.

The design of this experiment was to obtain six populations of cells (referred to for simplicity as “cell types”), differing only in their treatment with adenovirus and/or hypoxia, as shown below:

| “Cell | | | | |
|---|---|---|---|---|
| Type” | Adenovirus | Expressed gene | Oxygen condition | |
| 1 | AdApt ires-GFP | none | Normoxia | (20% Oxygen) |
| 2 | AdApt ires-GFP | none | Hypoxia | (0.1% Oxygen) |
| 3 | AdApt HIF-1α- | HIF-1α | Normoxia | (20% Oxygen) |
| | ires-GFP | | | |
| 4 | AdApt HIF-1α- | HIF-1α | Hypoxia | (0.1% Oxygen) |
| | ires-GFP | | | |
| 5 | AdApt EPAS1- | EPAS1 | Normoxia | (20% Oxygen) |
| | ires-GFP | | | |
| 6 | AdApt EPAS1- | EPAS1 | Hypoxia | (0.1% Oxygen) |
| | ires-GFP | | | |

Gene discovery can be implemented by comparing gene expression profiles between these “cell types”. According to conventional methods available in the literature, one would make comparisons between cell types 2 and 1. By implementing the present invention (Smartomics), several other possibilities are seen. Firstly, a comparison can be made between cell types 3 or 5 and cell type 1. Here, the stimulus of overexpressing key molecules involved in the hypoxia response may exceed the natural response the hypoxia, as seen for cell type 2. Secondly, in a preferred embodiment of the invention, a comparison can be made between cell types 4 or 6 and cell type 1. In this situation, the natural response to hypoxia is being augmented or boosted by overexpressing key molecules involved in the hypoxia response. It should be noted that the experimental design illustrated above uses a control adenovirus in place of untreated cells. By doing this, any non-specific effects of viral transduction should occur equally throughout the analysis, and will disappear.

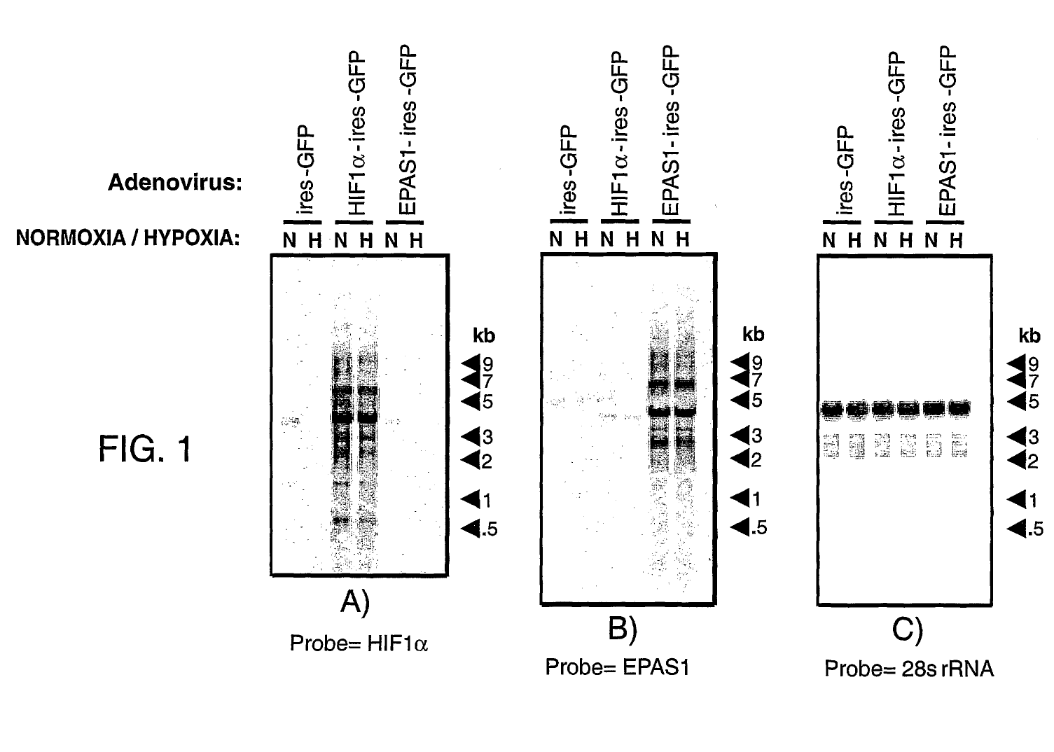

Although efficient adenoviral gene transfer was indicated by green fluorescence in the transduced macrophages, Northern blotting was used to confirm overexpression of HIF-1α and EPAS1. RNA samples extracted from cell types 1-6 as described above were analysed by Northern blotting (FIG. 1). The RNA samples (8 ug total RNA per lane) were electrophoresed on a formaldehyde denaturing 1% agarose gel, then transferred to a nylon membrane (Hybond-N, Amersham, UK), and sequentially hybridised with ³³P-labelled DNA probes complementary in nucleotide sequence to HIF-1α (FIG. 1a), EPAS1 (FIG. 1b) or 28S ribosomal RNA (FIG. 1c). The methodology used for Northern blotting, probe hybridisation under stringent conditions, and removal of probes between hybridisations, is well known in the art.

In FIG. 1a, it can be seen that all lanes contain a faint band of approximately 4 kb, corresponding to the endogenous HIF-1α mRNA. In lanes 3,4, which contain RNA from cells transduced with AdApt HIF-1α-ires-GFP, a much stronger band of a similar size is observed, indicating successful overexpression of HIF-1α.

In FIG. 1b, it can be seen that all lanes contain a very faint band of approximately 5 kb, corresponding to the endogenous EPAS1 mRNA. In lanes 5,6, which contain RNA from cells transduced with AdApt EPAS1-ires-GFP, a much stronger band at approximately 4 kb is observed, indicating successful overexpression of EPAS1. The difference in size of the endogenous and overexpressed EPAS1 is due to the long untranslated region of the endogenous gene, which is of no consequence.

In FIG. 1c, it can be seen that 28S ribosomal RNA is detected in all lanes, indicating equal loading of RNA on the gel.

By phosphorimager quantitative analysis of FIGS. 1a and 1b, it is apparent that overexpression levels of both HIF-1α and EPAS1 are approximately 80-fold over the endogenous levels. Adenoviral-directed mRNA overexpression of these genes is not further augmented by hypoxia. For example, in FIG. 1a, the band intensity for lane 4 does not exceed that for lane 3. However at the protein and functional levels, hypoxia potentiates the action of the proteins encoded by these mRNAs (Semenza GL. Annu Rev Cell Dev Biol. 1999;15:551-78. “Regulation of mammalian O2 homeostasis by hypoxia-inducible factor 1”).

Global mRNA expression profiles from the RNA samples isolated from the six “cell types” were obtained using Research Genetics Human GeneFilters Release 1 (GF200) (Research Genetics, Huntsville, Ala.). This method uses pre-made arrays of DNA complementary to 5,300 genes covering a range of levels of characterisation, including sequences which only match unannotated ESTs or cDNA sequences of unknown function.

The arrays are nylon in composition, and are spotted with DNA derived from specific IMAGE consortium cDNA clones (http://image.llnl.gov/image/). The arrays are hybridised to RNA samples which have been radioactively labelled with the isotope ³³P to measure the abundance of individual genes within the RNA samples. Multiple RNA samples are labelled and hybridised in parallel to separate copies of the array, and spot hybridisation signals are compared between the RNA samples.

Key issues in array-based mRNA expression analysis are sensitivity and reliability. Currently two other methods are available; glass microarrays and DNA chips, both of which utilise fluorescently labelled RNA (Bowtell D D. Nat Genet. 1999 January;21(1 Suppl):25-32. “Options available—from start to finish—for obtaining expression data by microarray.”). Although these methods are often believed to offer increased sensitivity over Nylon-based methods, this belief lacks definitive proof. To the contrary, a careful comparison of the three approaches shows that for similar amounts of unamplified RNA, the nylon-based radioactive method is superior (Bertucci F, Bernard K, Loriod B, Chang Y C, Granjeaud S, Birnbaum D, Nguyen C, Peck K, Jordan B R. Hum Mol Genet. 1999 September;8(9):1715-22. “Sensitivity issues in DNA array-based expression measurements and performance of nylon microarrays for small samples.”). The microarray and DNA chip methods require much larger amounts of RNA which are often not easily obtained from primary cells, or complicated amplification methods, which are liable to introduce error.

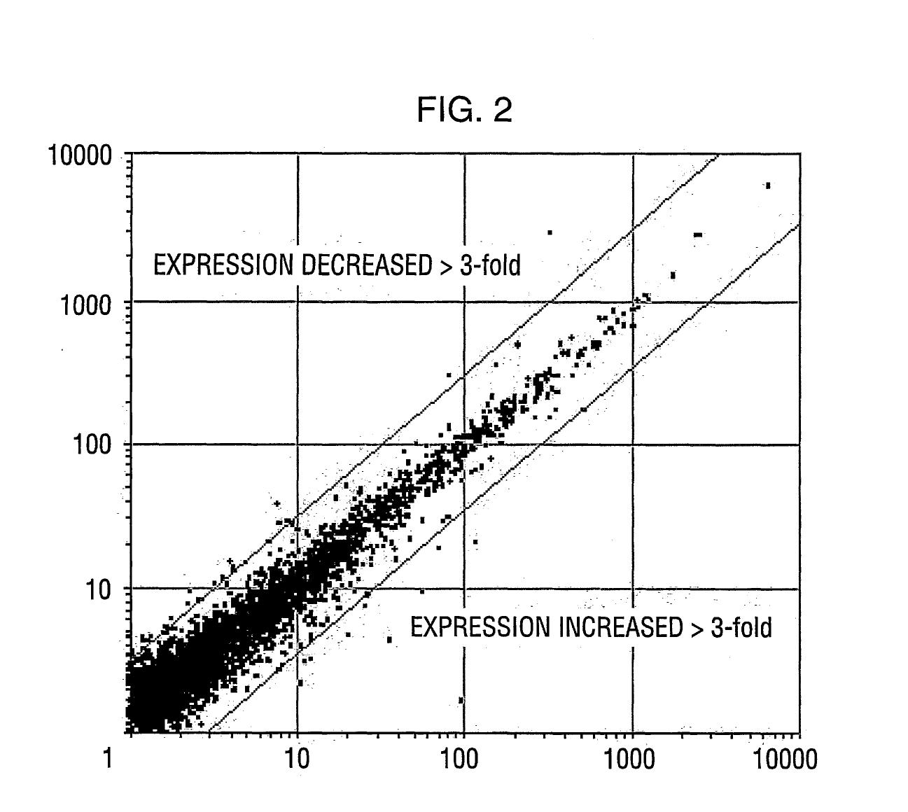

To demonstrate the sensitivity of the array-based gene expression method used in the current exemplification of Smartomics, a scatter plot of two representative RNA samples analysed in our laboratory using Research Genetics GeneFilters, demonstrates a range of detection approaching 4-logs (FIG. 2). By comparison, arguably the most sophisticated array-based method, the DNA chip, is quoted as having a range of detection of 3-logs (Affymetrix).

Therefore, it is reasonable to assume that the improvements afforded by Smartomics regarding sensitivity issues, as illustrated by the current exemplification, could not easily be obtained by utilising an alternative array-based method. In any case, any potentially superior array methodology could be further improved by utilising the Smartomics invention described here. An important utility of the present invention is that a high-throughput method such as array hybridisation can be used to identify expression changes which usually are only detectable by a very sensitive low throughput method such as RT-PCR or Northern blot.

RNA extracted from the 6 “cell types” as described above, was radioactively labelled and hybridised to separate copies of the Research Genetics Human GeneFilter GF200 (experiment #1). Methods provided by the manufacturer were followed (http://www.resgen.com/products/GF200_protocol.php3). Images of hybridised arrays were obtained using a Molecular Dynamics Storm phosphorimager. RNA was then stripped from the arrays, following the aforementioned protocol.

To ensure reproducibility, this procedure was repeated with the same RNA samples (experiment #2). The entire data set was then imported and analysed using Research Genetics Pathways 3.0 software, as explained in the Pathways 3.0 manual. Key aspects of the current analysis are summarised below:

Project Tree Set-Up

“Condition Pairs” mode was used to simultaneously analyse multiple experiments. “Condition” means several arrays hybridised to similar RNA samples, derived from the same “cell type”.

| Condition | “Cell Type” | Adenovirus | Oxygen | Experiment # |
|---|---|---|---|---|
| 1 | 1 | AdApt ires-GFP | Normoxia | 1 |
| 1 | 1 | AdApt ires-GFP | Normoxia | 2 |
| 2 | 2 | AdApt ires-GFP | Hypoxia | 1 |
| 2 | 2 | AdApt ires-GFP | Hypoxia | 2 |
| 3 | 3 | AdApt HIF-1α- | Normoxia | 1 |
| | | ires-GFP | | |
| 3 | 3 | AdApt HIF-1α- | Normoxia | 2 |
| | | ires-GFP | | |
| 4 | 4 | AdApt HIF-1α- | Hypoxia | 1 |
| | | ires-GFP | | |
| 4 | 4 | AdApt HIF-1α- | Hypoxia | 2 |
| | | ires-GFP | | |
| 5 | 5 | AdApt EPAS1- | Normoxia | 1 |
| | | ires-GFP | | |
| 5 | 5 | AdApt EPAS1- | Normoxia | 2 |
| | | ires-GFP | | |
| 6 | 6 | AdApt EPAS1- | Hypoxia | 1 |
| | | ires-GFP | | |
| 6 | 6 | AdApt EPAS1- | Hypoxia | 2 |
| | | ires-GFP | | |

Normalisation Set-Up

The “all data points” option and Y. Chen algorithm with default settings were selected, as explained in the Pathways 3.0 manual. The two experiments were treated as separate normalisation groups, such that global differences between hybridisation signals from different arrays from the same experiment were corrected.

Comparison Analysis

| | |
|---|---|
| Pair-wise comparisons were made between | condition 2 and condition 1 |
| | condition 3 and condition 1 |
| | condition 4 and condition 1 |
| | condition 5 and condition 1 |

In other words, pair-wise comparisons were made using condition 1 (i.e. cell type 1) as the reference condition. This corresponds to cells transduced with the control adenovirus AdApt ires-GFP and placed under normal oxygen concentration (normoxia). Comparisons are made in this way for all genes present on the Research Genetics GF200 array. By comparing conditions, the analysis considers data from both experiments #1 and #2.

Filter Settings

Filtering was then done to select genes with expression ratios of above 2.0 for at least one of the five pair-wise comparisons detailed above. Genes with low signal intensities for all of the six conditions were automatically eliminated, using an Intensity II filter of min 0.2, max 1000. Genes that did not respond in a reproducible way in experiment #1 and #2, were automatically eliminated using the Students t-test filter (90% confidence level).

Results were output as expression profiles of individual genes, showing normalised signal intensity and expression ratio. A key advantage of analysis in Pathways 3.0 is that high magnification thumbnail images of individual spots are displayed. This allows visual verification that the area being measured truly covers the region containing the hybridised array spot, and that the spot is real and not a background artefact.

Minor differences between quantitative data and corresponding thumbnail images are sometimes seen even though the sampled area is clearly the bona fide array spot. For example, by eye there might seem to be a small difference between two spots, though the quantitative analysis might suggest a larger difference. It should be noted that thumbnail images are not normalised to compensate for global differences, and are limited in image quality. Greyscale images are inherently limited in their capacity to depict quantitative differences in intensity. Digital images generated by the Storm phosphorimager cover a linear dynamic range of 100,000 for a single pixel, whereas printed images can only be depicted as 256 shades of grey.

Results for Three Representative Known Hypoxia-Regulated Genes

As demonstration that overexpression of HIF-1α or EPAS1 together with hypoxia exposure is superior to using non-transduced hypoxic cells, in terms of discovering bona fide hypoxia-regulated genes, results are shown for genes which are already known in the art to be regulated in hypoxia.

Three genes have been selected which are represented as double spots on the Research Genetics GF200 array. Therefore, because the whole experiment was repeated, a total of four repeat comparisons are possible for these genes.

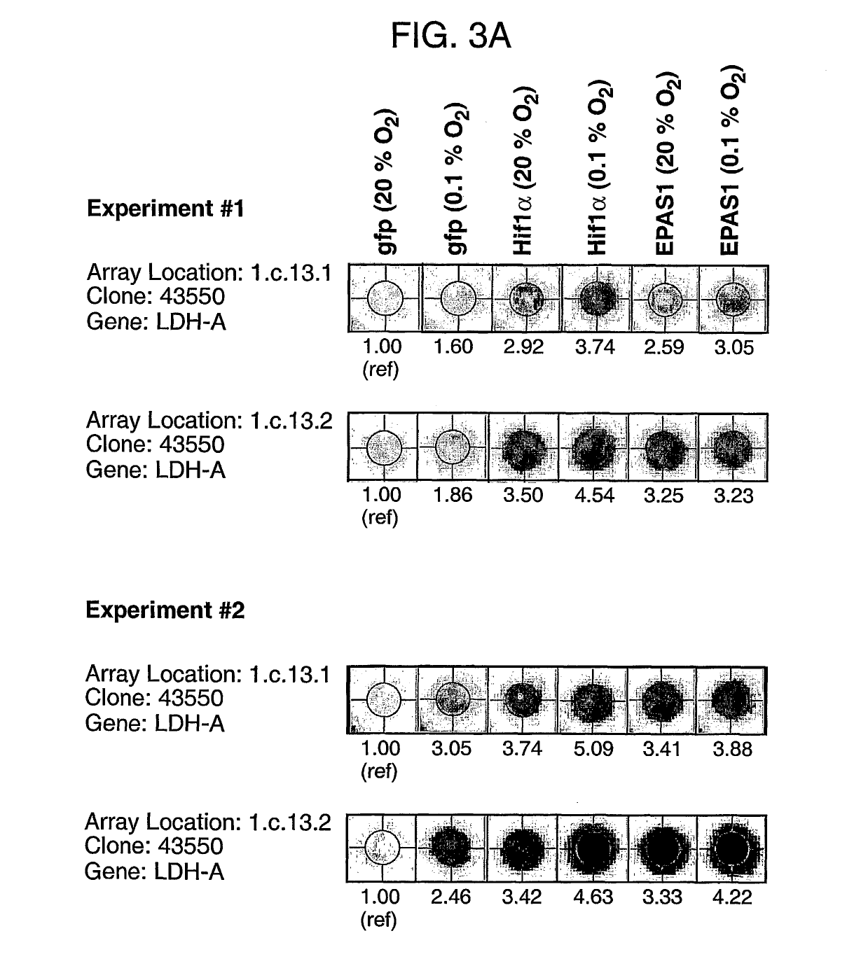

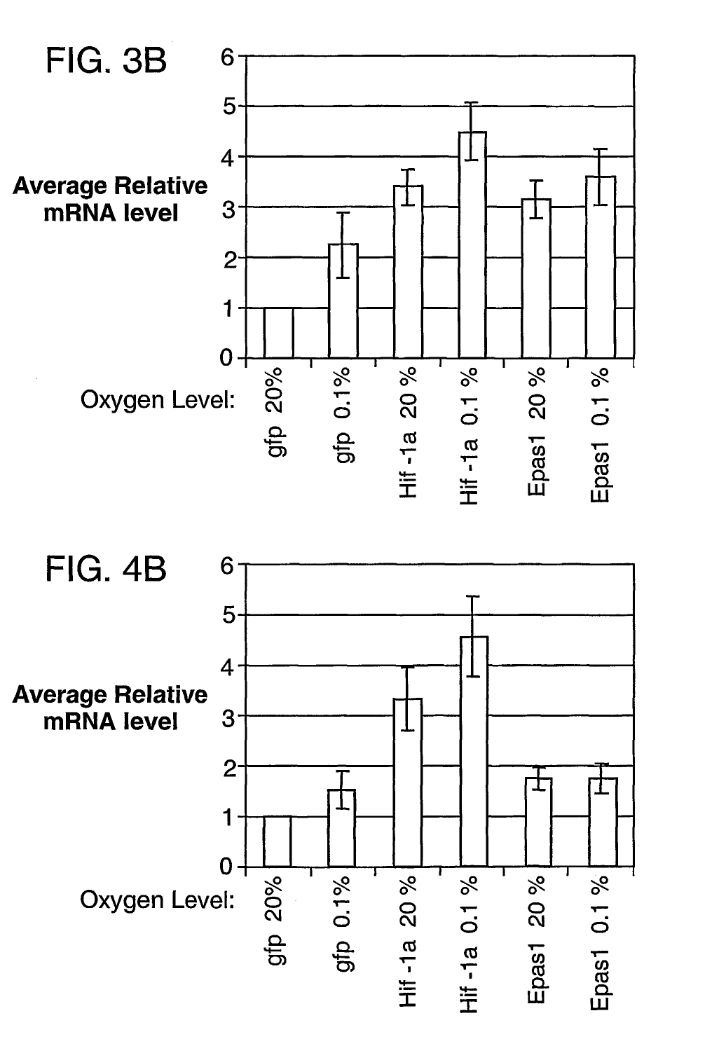

The lactate dehydrogenase A (LDH-A) gene is known in the art to be activated by hypoxia (Webster K A. Mol Cell Biochem. 1987 September;77(1):19-28. “Regulation of glycolytic enzyme RNA transcriptional rates by oxygen availability in skeletal muscle cells.”). In FIG. 3, it can be seen that in response to hypoxia alone (gfp 0.1% O₂) there is on average a 2.24-fold increase in mRNA expression compared to normoxia (gfp 20% O₂).

By overexpressing HIF-1α there is on average a 3.39-fold increase in LDH-A expression, providing a significant improvement over the natural response (FIG. 3; HIF-1α 20% O₂). By utilising a preferred embodiment of the Smartomics method, and simultaneously overexpressing HIF-1α in the presence of hypoxia, the average response of LDH-A is elevated further to 4.50-fold (FIG. 3; HIF-1α0.1% O₂).

In the prior art it has been established that HIF-1α is responsible for mediating the hypoxia-induced activation of LDH-A (Iyer N V, Kotch L E, Agani F, Leung S W, Laughner E, Wenger R H, Gassmann M, Gearhart J D, Lawler A M, Yu A Y, Semenza G L. Genes Dev. Jan. 15, 1998; 12(2):149-62 “Cellular and developmental control of O2 homeostasis by hypoxia-inducible factor 1 alpha.”). However it has never been envisaged or demonstrated that overexpression of HIF-1α in a stable manner using viral gene transfer techniques, both with or without simultaneous hypoxia, causes secondary changes in gene expression which are markedly greater than the natural hypoxia response. The response to hypoxia of LDH-A is also improved by overexpressing EPAS1 (FIG. 3; EPAS1), though this is less dramatic than overexpressing HIF-1α.

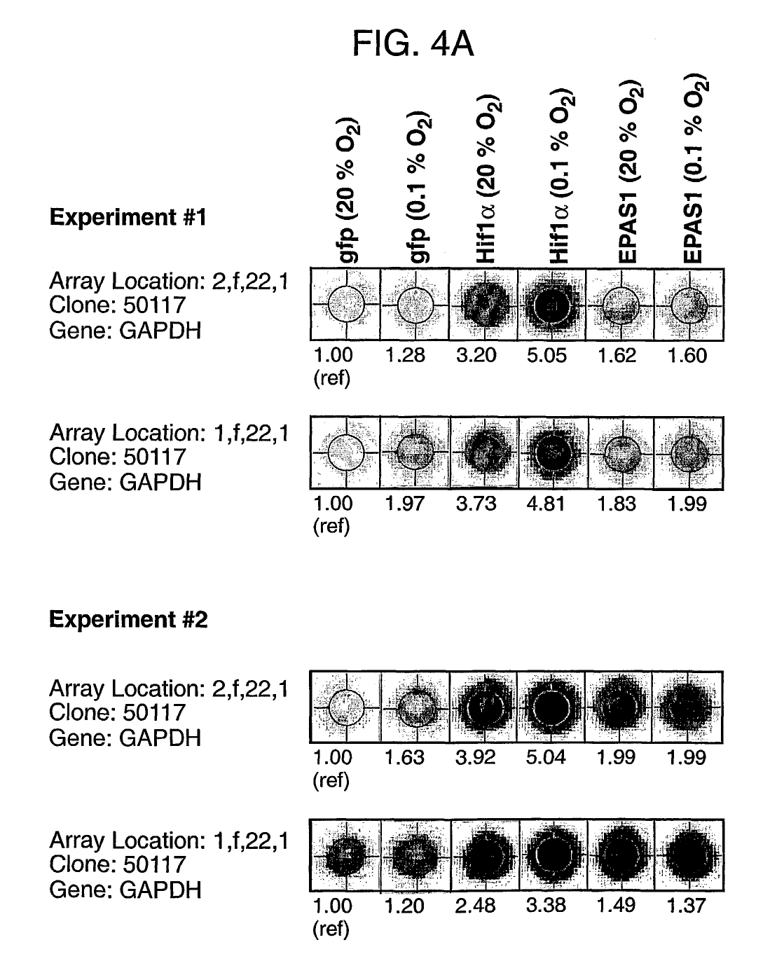

Like LDH-A, the glyceraldehyde 3-phosphate dehydrogenase (GAPDH) gene is known in the art to be activated by hypoxia (Webster K A. Mol Cell Biochem. 1987 September;77(1):19-28. “Regulation of glycolytic enzyme RNA transcriptional rates by oxygen availability in skeletal muscle cells.”). In FIG. 4, it can be seen that in response to hypoxia alone (gfp 0.1% O₂) there is on average a 1.52-fold increase in mRNA expression compared to normoxia.

By overexpressing HIF-1α there is on average a 3.33-fold increase in GAPDH expression, providing a significant improvement over the natural response g(FIG. 4; HIF-1α 20% O₂). By utilising the full embodiment of the Smartomics method, and simultaneously overexpressing HIF-1α in the presence of hypoxia, the average response of GAPDH is elevated further to 4.57-fold (FIG. 4; HIF-1α 0.1% O₂).

In the published literature, it has been established that HIF-1α is responsible for mediating the hypoxia-induced activation of GAPDH (Iyer N V, Kotch L E, Agani F, Leung S W, Laughner E, Wenger R H, Gassmann M, Gearhart J D, Lawler A M, Yu A Y, Semenza G L. Genes Dev. Jan. 15, 1998; 12(2):149-62 “Cellular and developmental control of O2 homeostasis by hypoxia-inducible factor 1 alpha.”). However in the art, it has never been envisaged or demonstrated that overexpression of HIF-1 Ic in a stable manner using viral gene transfer techniques, both with or without simultaneous hypoxia, causes secondary changes in gene expression which are markedly greater than the natural hypoxia response.

For GAPDH, it can be seen that overexpression of EPAS1 (FIG. 4; EPAS1 20% O₂ and 0.1% O₂), has a significantly smaller effect than overexpressing HIF-1α. This demonstrates a separate embodiment of the Smartomics method, whereby genes are identified which respond selectively or preferentially to overexpression of EPAS1 or HIF-1α.

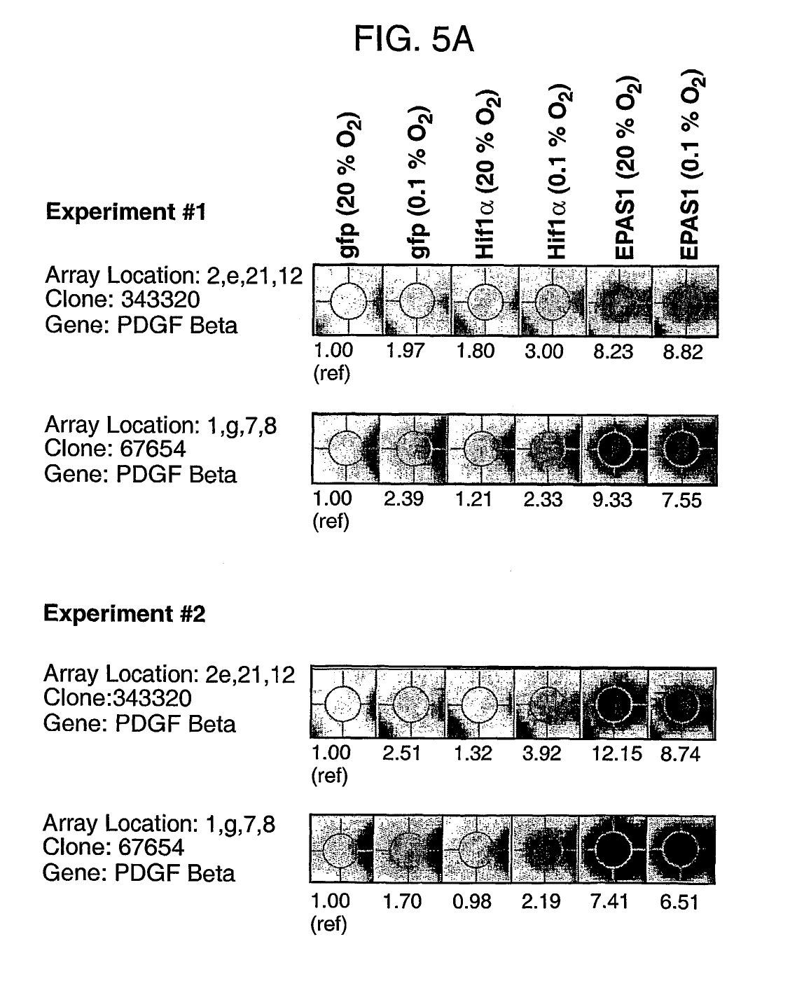

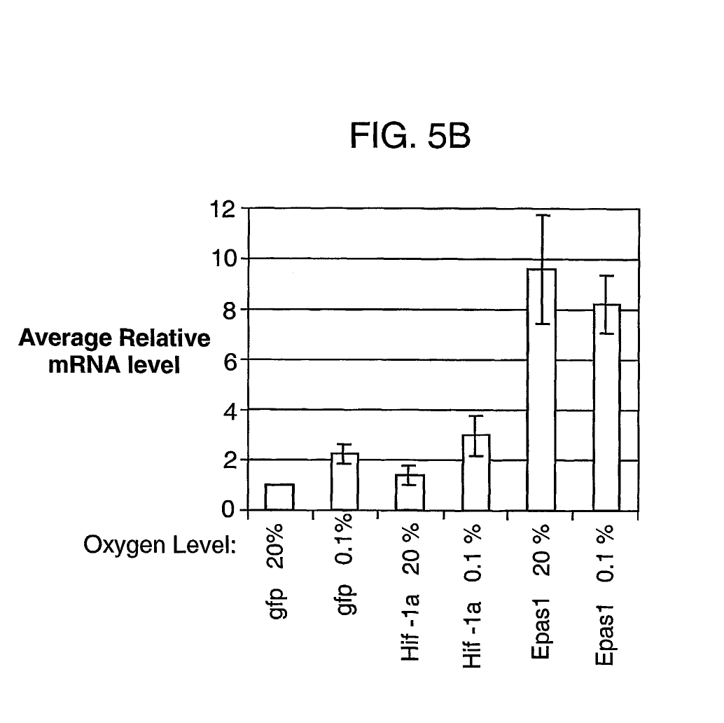

Platelet derived growth factor beta (PDGF β) is also known in the art to be activated by hypoxia (Kourembanas S, Hannan R L, Faller D V. J Clin Invest. 1990 August;86(2):670-4 “Oxygen tension regulates the expression of the platelet-derived growth factor-B chain gene in human endothelial cells.”). In FIG. 5, it can be seen that in response to hypoxia alone (gfp 0.1% O₂) there is on average a 2.14-fold increase in mRNA expression compared to normoxia.

By overexpressing EPAS1, there is on average a 9.28-fold increase in PDGF β expression (FIG. 5; EPAS1 20% O₂), providing a large improvement over the natural response. In this case, the combination of hypoxia and EPAS1 overexpression does not exceed the response of EPAS1 overexpression alone, indicating saturation of the dose-response (FIG. 5; EPAS1 0.1% O₂).

From FIG. 5, it is clear that there is a striking specificity in the response of PDGF β to EPAS1 and HIF-1α, in the opposite manner observed for GAPDH. Overexpression of HIF-1α alone has no significant effect on PDGF β, whereas overexpression of EPAS1 produces large effects. This demonstrates a separate embodiment of the Smartomics method, whereby genes are identified which respond selectively or preferentially to overexpression of different factors which act in the same pathway.

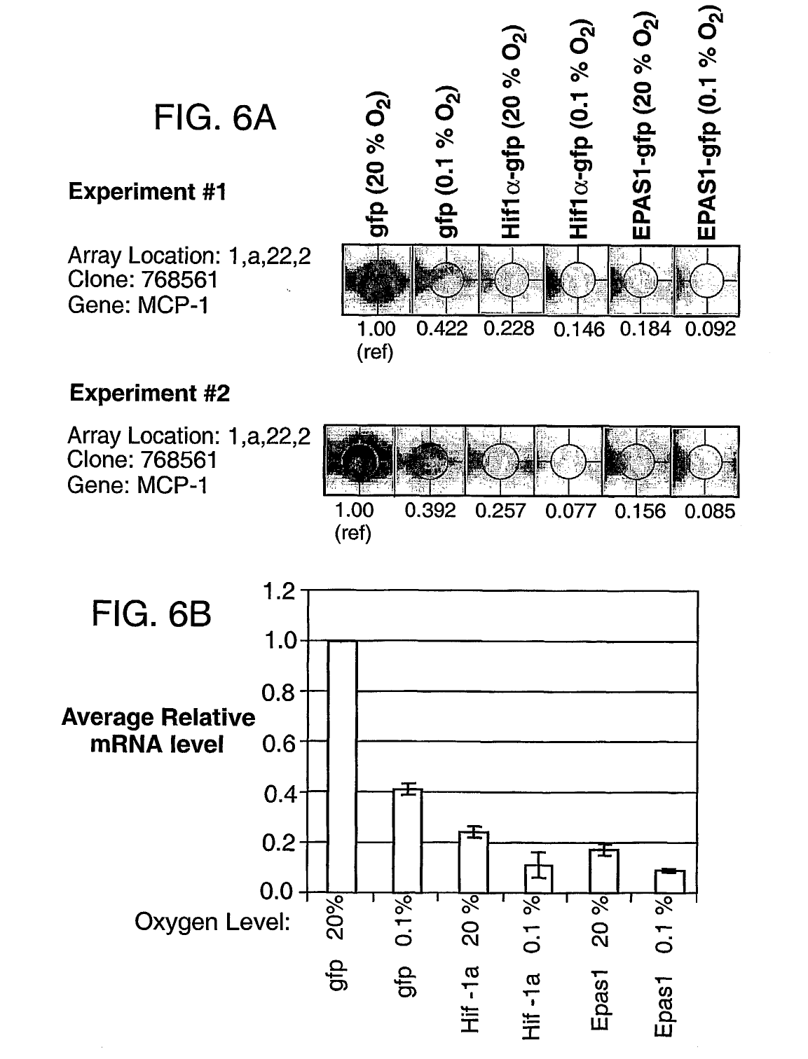

The gene encoding monocyte chemotactic protein 1 (MCP-1) is known in the art to respond to hypoxia in a negative fashion, by decreasing mRNA expression (Negus R P, Turner L, Burke F, Balkwill F R. J Leukoc Biol. 1998 June;63(6):758-65. “Hypoxia down-regulates MCP-1 expression: implications for macrophage distribution in tumors”). In FIG. 6 it can be seen that in response to hypoxia alone (gfp 0. 1% O₂) there is on average a 0.407-fold change (i.e. a 2.46 fold decrease) in mRNA expression compared to normoxia.

By overexpressing HIF-1α, there is on average a 0.243-fold change (i.e. a 4.11-fold decrease) in MCP-I expression, providing a significant improvement over the natural response (FIG. 6; HIF-1α 20% O₂). By utilising a preferred embodiment of the Smartomics method, and simultaneously overexpressing HIF-1α in the presence of hypoxia, the average response of MCP-1 is further improved to a 0.112-fold change (i.e. an 8.93-fold decrease) (FIG. 6; HIF-1α0.1% O₂). Even more pronounced improvements in the hypoxia-induced inhibition of MCP-1 expression are obtained by overexpressing EPAS1 (FIG. 6; EPAS1 20% O₂ and 0.1% O₂). This demonstrates a use of Smartomics to improve the discovery of genes that are inhibited or repressed by disease signals.

The finding that overexpressing HIF-1α or EPAS1 potentiates hypoxia-induced gene repression, as exemplified by MCP-1, is totally without precedent in this field. The stricture of both HIF-1α and EPAS1 proteins is that they contain transactivation domains but not known transcriptional repressor domains (Pugh C W, O'Rourke J F, Nagao M, Gleadle J M, Ratcliffe P J. J Biol Chem. Apr. 25, 1997; 272(17):11205-14. “Activation of hypoxia-inducible factor-1; definition of regulatory domains within the alpha subunit.”).

The results explained above relate to an array gene expression analysis, in which over 50 genes were identified as being regulated in hypoxia, from a total set of approximately 5300 genes on the array. By focusing on genes known in the art to be regulated in hypoxia, and showing how the Smartomics method can significantly enhance the response, an argument is provided that Smartomics would provide an improved method for the identification of novel bona fide hypoxia-regulated genes. In the current study, this can also be shown directly, for novel genes which were discovered using the Smartomics method, as presented below. Because expression changes arising from a conventional analysis are also covered in this analysis (i.e. hypoxia/normoxia comparisons without viral overexpression), the advantage of the Smartomics invention is clearly demonstrated.

Table 1 lists unannotated genes or ESTs which were identified in this analysis as being activated in response to viral-directed overexpression, but which would not have been identified from a hypoxia/normoxia comparison as done in the prior art. The final five columns of Table 1 show expression ratios compared to cells transduced with AdApt-ires-GFP in normoxia. The first of these five columns is the response without Smartomics, and in all cases shown here, the levels are below significance. The other four columns represent results obtained using the present invention, and significant responses are seen here. In particular, in the final rows of this table, novel genes are identified which show large responses to EPAS1 overexpression.

TABLE 1
Novel Genes Identified By Smartomics

| | NUCLEOTIDE | | PROTEIN | | RATIO (compared to gfp N) | | | | |
|---|---|---|---|---|---|---|---|---|---|
| Title | Seq ID | Accession | Seq ID | Accession | gfp H | hif N | hif H | epas N | epas H |
| ESTs, Moderately similar to | | N68173 | | none | 0.85 | 2.44 | 1.85 | 1.67 | 1.66 |
| AF119917 63 PRO2831 | | | | | | | | | |
| ESTs | | H82330 | | none | 1.06 | 1.11 | 0.90 | 1.88 | 2.79 |
| ESTs | | T97204 | | none | 1.25 | 1.20 | 0.84 | 2.03 | 2.76 |
| ESTs | | R25464 | | none | 0.96 | 1.51 | 1.41 | 2.15 | 3.01 |
| ESTs | | R25464 | | none | 1.12 | 1.70 | 1.35 | 2.23 | 2.92 |
| ESTs | | R95132 | | none | 0.91 | 1.38 | 1.06 | 2.32 | 2.79 |
| ESTs, Weakly similar to | | N80371 | | none | 1.70 | 1.26 | 2.02 | 2.07 | 1.87 |
| A49134 Ig kappa chain V-I | | | | | | | | | |
| region | | | | | | | | | |
| ESTs | | R09498 | | none | 1.06 | 1.73 | 1.53 | 1.94 | 2.18 |
| PRO0518 hypothetical | | R11658 | | AAF69617 | 0.89 | 1.11 | 0.97 | 3.81 | 3.89 |
| protein | | | | | | | | | |
| ESTs | | N74648 | | none | 0.94 | 0.78 | 1.01 | 3.39 | 3.13 |
| ESTs | | T86016 | | none | 1.42 | 1.73 | 1.59 | 3.78 | 3.65 |
| ESTs | | N99839 | | none | 0.98 | 2.02 | 1.46 | 2.88 | 3.91 |
| hypothetical protein | | R02569 | | AAF64262 | 1.13 | 1.31 | 1.32 | 2.92 | 2.63 |
| LOC51317 | | | | | | | | | |
| ESTs | | R06745 | | none | 1.00 | 2.17 | 1.77 | 3.00 | 2.59 |
| ESTs, Highly similar to | | R00332 | | BAB15101 | 1.71 | 1.41 | 1.58 | 6.79 | 6.45 |
| A53770 | | | | | | | | | |
| ESTs | | N64734 | | none | 1.44 | 0.97 | 1.36 | 9.50 | 10.29 |
| ESTs | | T85201 | | none | 0.87 | 1.18 | 1.06 | 14.99 | 14.71 |

Column 1 is the gene title as used in the UniGene database on 16 Feb. 2001. Nucleotide and protein acessions are from the Genbank database. The final five columns show expression levels expressed as a ratio compared to cells transduced with AdApt ires-GFP in normoxia. gfp H: Expression in cells transduced with AdApt ires-GFP in hypoxia. Hif N: Expression in cells transduced with AdApt Hif-1α-ires-GFP in normoxia. Hif H: Expression in cells transduced with AdApt Hif-1α-ires-GFP in hypoxia. EPAS N: Expression in cells transduced with AdApt Epas1-ires-GFP in normoxia. EPAS H: Expression in cells transduced with AdApt Epas1-ires-GFP in hypoxia.

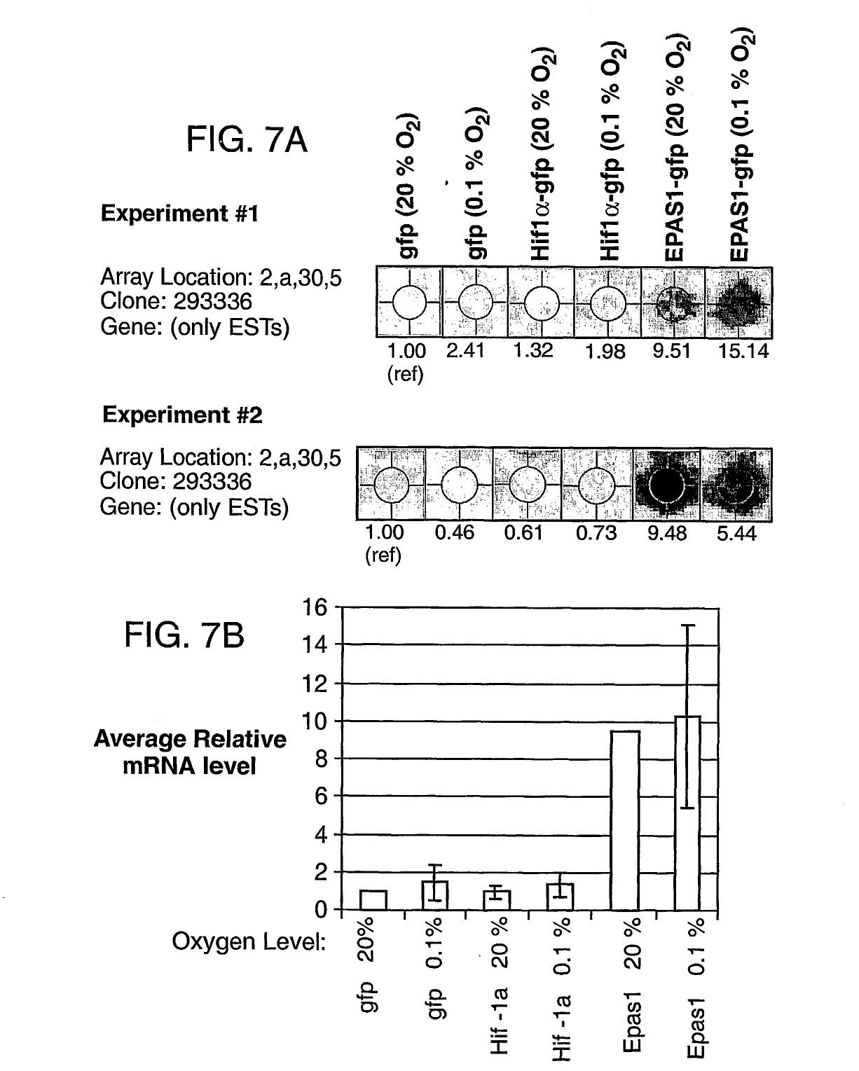

FIG. 7 shows the expression profile of one of these genes, corresponding to an EST (GenBank accession N64734; IMAGE clone 293336). In the UniGene EST database (http://www.ncbi.nlm.nih.gov/UniGene/) this EST is currently clustered with only two other ESTs with accessions AI051607 (IMAGE 1674154) and T87161 (IMAGE 293336). The UniGene cluster number is Hs.16335, and it is totally unannotated in the database. Sequence analysis shows that this rare sequence is incomplete and lacks information on the protein coding sequence. In the Ensembl database of human genome project gene annotation (http://www.ensembl.org/) blast searches of predicted or confirmed cDNA sequences do not identify this EST. It is therefore apparent that from public domain information, the gene corresponding to EST IMAGE 293336, is a truly novel and unannotated gene.

In FIG. 7, thumbnail array spot images are shown at maximal contrast, such that the background signal is apparent. It can be seen that in response to hypoxia alone (gfp 0.1% O₂) there is on average a 1.4-fold increase in mRNA expression compared to normoxia. However, this is not significant, because it is derived from widely different ratios from individual experiments (2.41 and 0.46). From the thumbnail images for gfp 20% O₂ and gfp 0.1% O₂ it is evident that expression of the genes under these conditions is below the detection threshold of the array-based method. However, when the Smartomics invention is used, and EPAS1 is overexpressed using viral gene transfer methods, a clearly detectable response in seen, with induction ratios of over 8-fold (FIG. 7; EPAS1 20% O₂ or 0.1% O₂). The expression profile in FIG. 7 also demonstrates a separate embodiment of Smartomics, for the identification of genes which respond selectively to HIF-1α or EPAS1.

To confirm the results presented in FIG. 7, a more sensitive method was used to study expression of the gene corresponding to IMAGE clone 293336, namely virtual Northern blotting. It should be noted that this method would not have been suitable for the original discovery that IMAGE clone 293336 is induced by hypoxia, because virtual Northern blotting and similar methods do not allow simultaneous screening of large numbers of genes. The technique is similar to conventional Northern blotting, with the exception that double stranded cDNA corresponding to the mRNA population of expressed genes is resolved by electrophoresis and blotted onto a nylon membrane. It relies on a method of cDNA synthesis which produces full length cDNA molecules, which is commercially available (SMART PCR cDNA Synthesis Kit; Clontech Laboratories Inc, Palo Alto, Calif., USA).

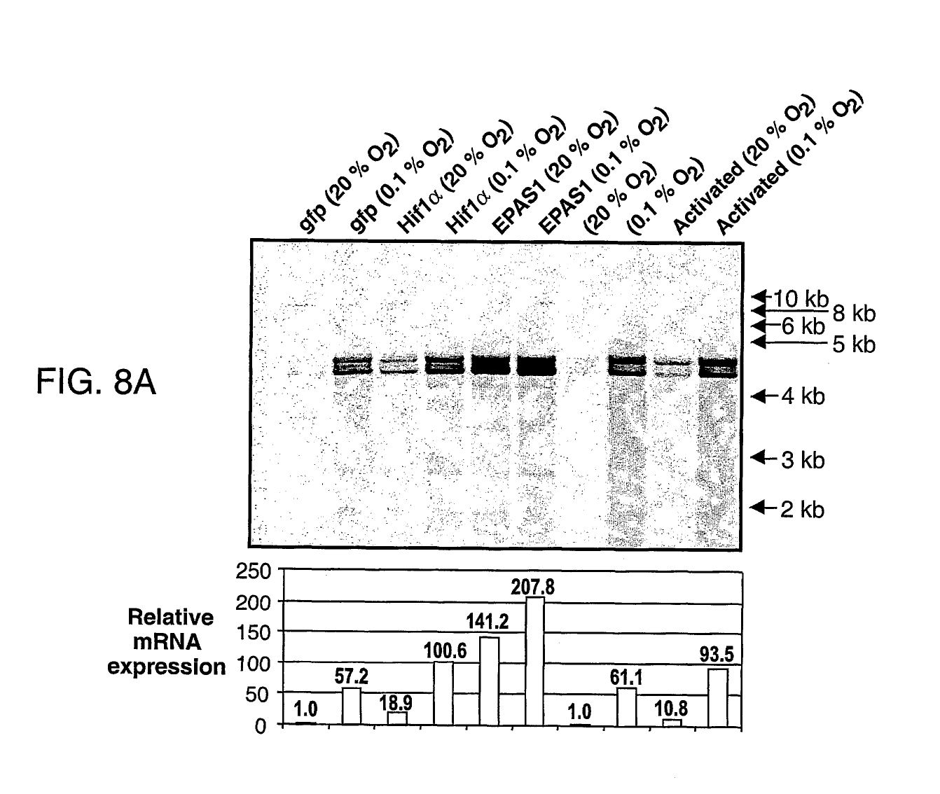

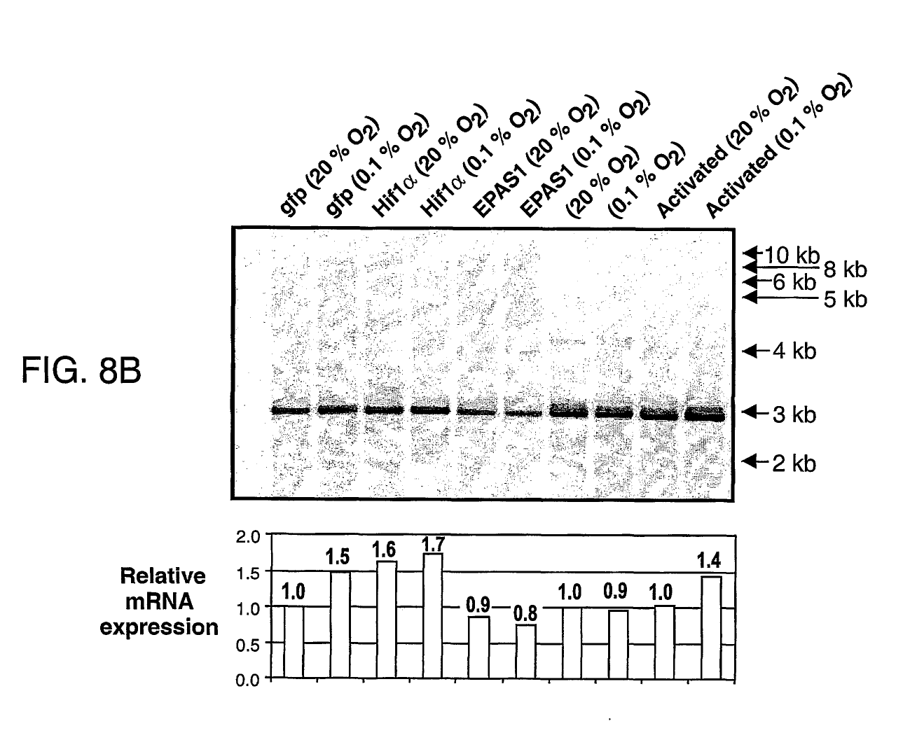

The method for virtual Northern blotting was followed as described in the instruction manual for the SMART PCR cDNA Synthesis Kit. Briefly, 600 ng cDNA was synthesised from the six RNA samples used for array hybridisation. An additional four RNA samples were also processed, derived from non-transduced macrophages cultured in normoxia and hypoxia (6 hours at 0.1 % O₂) both with and without pre-treatment for 16 hours with 100 ng/ml Lipopolysaccharide (E.coli 026:B6 Sigma, UK) and 1000 u/ml human gamma interferon (Sigma, UK). This combination of factors causes macrophage activation, a process key to the physiological and pathophysiological actions of the macrophage. All 10 cDNA samples were resolved on an agarose gel, and alkali transfer onto Hybond N+ membrane (AmershamPharmacia, UK) was carried out according to the Hybond N+ instructions. Stringent hybridisations with ³³P-labelled cloned cDNA probes were performed as for standard Northern blot hybridisation, which is well known in the art. cDNA probes were radiolabelled using a commercially available kit (Prime-a-Gene, Promega, UK). The virtual Northern blot was hybridised first with the cDNA insert of IMAGE clone 1674154 from UniGene cluster Hs.16335 (FIG. 8a). The blot was then stripped, by a high temperature/low salt wash, and was re-probed with the protein coding region of the human β-actin gene (FIG. 8b).

From FIG. 8a, it can be seen that the mRNA corresponding to Hs.16335 is detected as a doublet band of approximately 4.5 kb. This gene is strongly induced by adenoviral-directed overexpression of EPAS1 (lanes 5,6), consistent with the array data from FIG. 7. The higher induction ratios in this non-array analysis are due to increased sensitivity afforded by the virtual Northern technique. Unlike the array data, expression of Hs.16335 is within the range of detection for all RNA samples. Importantly, hypoxia alone is seen to cause an induction ratio of approximately 60-fold (FIG. 8a; lanes 2, 8). Therefore Hs.16335 is identified as a bone fide hypoxia-regulated gene, despite being beneath the detection level of an array screen in the absence of the present invention (Smartomics).

The results in FIG. 8a also demonstrate a separate embodiment of the Smartomics method, whereby genes are identified which respond selectively or preferentially to overexpression of EPAS1 or HIF-1α. Overexpression of HIF-1α causes an induction ratio of 18.9-fold (lane 3), whereas overexpression of EPAS1 causes a much larger induction ratio of 141-fold (lane 5).

In FIG. 8a lane 9, it is shown that activation of macrophages by LPS and TNFα causes a 10.8-fold increase in expression of the gene corresponding to Hs.16335. Therefore this novel gene is possibly relevant to the inflammatory functions of macrophages.

In FIG. 8b expression of the human β-actin gene is found to be roughly constant throughout this experiment, consistent with the differences in FIG. 8a being due to specific changes in gene expression.

Rapid amplification of cDNA ends (RACE) may be performed to clone the full length version of the gene corresponding to Hs.16335, based on the size of the cDNA size on the virtual Northern blot. Sequencing and functional analysis of this gene will possibly lead to the identification of a new therapeutic target molecule. Crucial to this process was the initial use of the Smartomics invention.

### Example 3

EIAV Vector Construction

This example describes the generation of an EIAV vector (pONY8.1SM) with four unique cloning sites downstream of a CMV promoter. pONY8.1SM is the most minimal EIAV vector to date in terms of EIAV sequence that it contains (˜1.1 kb) and EIAV proteins it expresses (none). The vector is an example of a gene transfer system that could be used in a differential expression screening method according to our invention. However, other gene transfer systems based on any other lentivirus, retrovirus, herpesvirus, adenovirus, alphavirus, adeno-associated virus, herpes virus or DNA in any appropriate formulation, could be used.

Construction of EIAV-Based Vector pONY8.1SM

The starting point was pONY4.0Z (GB9727135.7 and Mitophanous et al., 1999). The first two ATG triplets in the EIAV gag region were replaced with ATTG to eliminate the expression of gag from the EIAV genome while maintaining gag sequences in the vector. The gag sequence was found to be important for maintaining high titre vector production.

The ATG to ATTG change was carried out by PCR. Primers ATTG1 and PS2 were used to PCR amplify the EIAV leader/gag sequence. The template for this was the plasmid pONY3.1 (GB9727135.7 and Mitophanous et al., 1999). This PCR fragment contains a Nar I and Xba I site at the 5′ and 3′ ends respectively. This fragment was inserted into pONY4Z cut with a Nar I and Xba I to produce pONY8.0Z.

ATTG1 Primer:
AGTTGGCGCCCGAACAGGGACCTGAGAGGGGCGCAGACCCTACCTGTTGA
ACCTGGCTGATCGTAGGATCCCCGGGACAGCAGAGGAGAACTTACAGAAG
TCTTCTGGAGGTGTTCCTGGCCAGAACACAGGAGGACAGGTAAGATTGGG
AGACCCTTTGACATTGGAGCAAGGCGCTCAAGAA
Underlined = Nar I site
PS2 primer:
TAGTTCTAGAGATATTCTTCAGAG
Underlined = Xba I site

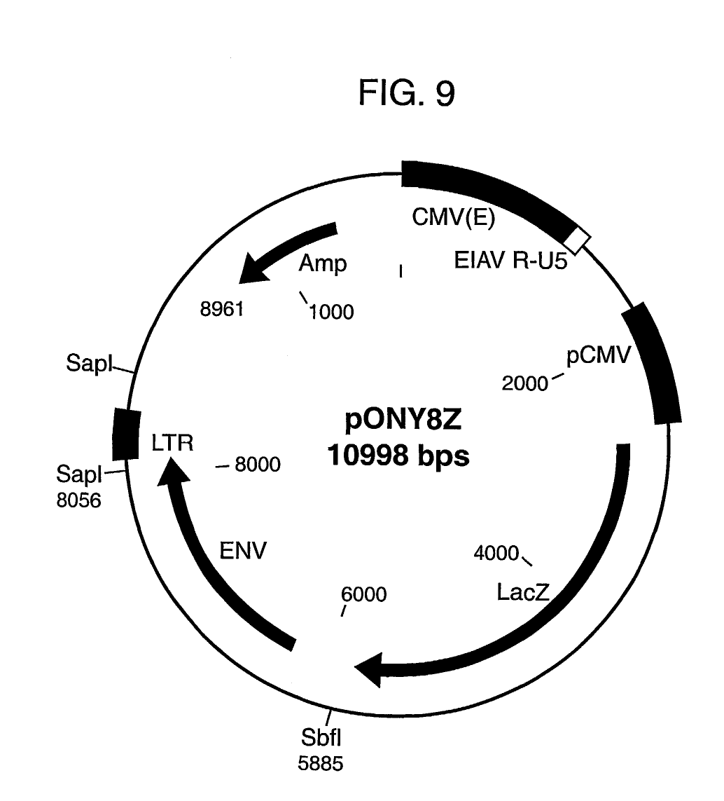

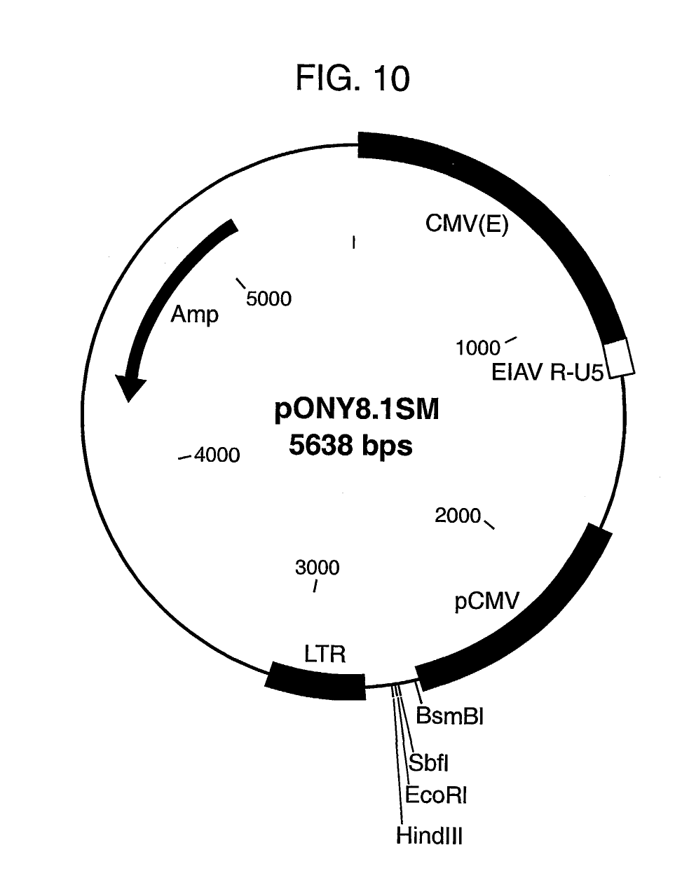

pONY8.1SM is an EIAV vector genome containing an internal CMV promoter from which any gene of interest is expressed. It was made by deleting a part of the env sequence from pONY8Z. pONY8Z was cut with Sbf I (position 5885). This was then partially cut with Sap I (there are two Sap I sites in pONY8Z, see FIG. 9). The molecule cut at site 8056 was then purified, blunt ended and re-ligated to give pONY8.1Z. To generate pONY8.1SM pONY8.1Z was cut with Sac II and Sph I, blunt ended and re-ligated. This removes the lacZ gene and creates 4 unique sites, Bsm BI, Sbf I, Eco RI and Hind III (FIG. 10) for the insertion of any gene or library of genes. Sbf I has an 8 base recognition sequence which makes it useful for inserting unknown genes.

### Example 4

Generation of EIAV Vector that Expresses HIF1-α

This example describes the generation of an EIAV vector (pONY8.1SMHIF1) that is able to express HIF-1α from an internal CMV promoter. The accession number for human HIF-1α is U22431. To make pONY8.1SMHIF1 HIF-1α was PCR amplified from cDNA generated from mRNA isolated from Jurkat cells. The primers for this were HIFPM1 and HIFPM2 described below. They contain Sbf I sites for cloning and the Kozak sequence has been used to enhance translation. The PCR product generated this way contains Sbf I cloning sites flanking the HIF-1α open reading frame. This was cut with Sbf I and inserted into pONY8.1SM cut with Sbf I. The plasmid generated this way was called pONY8.1SMHIF1.

HIFPM1 Primer: ATCGCCTGCAGGCCACCATGGAGGGCGCCGGCGGCGCG

Sbf I site=underlined, Kozak sequence=bold and italics, ATG start codon=underlined and italics

HIFPM2 Primer: ACTGCCTGCAGGTCAGTTAACTTGATCCAAAGCTCTGAG

Sbf I site=underlined

This plasmid is used in conjunction with gag-pol and env expressing plasmids to produce EIAV-based vector particles as described in Mitrophanous et al., 1999. These particles are then used to transduce a variety of cell types that may be of interest in the context of genes controlled directly or indirectly by the Hifl pathway.

One example is primary human skeletal muscle cells. Transduced and untransduced cell populations are compared. In addition transduced cells in low oxygen concentrations are compared with untransduced cells in normal oxygen concentrations.

RNA samples are prepared for the analysis of differential gene expression. These are labelled either radioactively or fluorescently, and hybridized to arrays of cDNAs on solid supports. Genes which are upregulated by hypoxia and/or expression of individual HIF proteins produce quantitatively stronger hybridization signals. Array strategies may involve either nylon or glass supports, which are reviewed in Bowtell, 1999. Details of methodologies involved in the glass support approach are detailed Eisen and Brown, 1999. Here, fluorescently labelled probes are used and hybridization is detected using a laser confocal scanner. For the Nylon support approach, standard molecular biology methods of dot blotting and hybridization are involved as detailed in Molecular Cloning: A laboratory manual Sambrook, J et al, Cold Spring Harbor Laboratory Press. Here, RNA samples to be compared are radioactively labelled and hybridization is detected using a phosphorimager.

Arrays can be purchased from Research Genetics, Huntsville, Ala. or would be fabricated in-house using cDNA clones generated by subtraction cloning (PCR-Select method, owned by Clontech Palo Alto, Calif.). Fabrication would involve use of an arraying robot (MicroGrid, BioRobotics Ltd, Cambridge, UK).

### Example 5

Generation of Codon-Optimised EIAV Vector Expressing HIF1-α

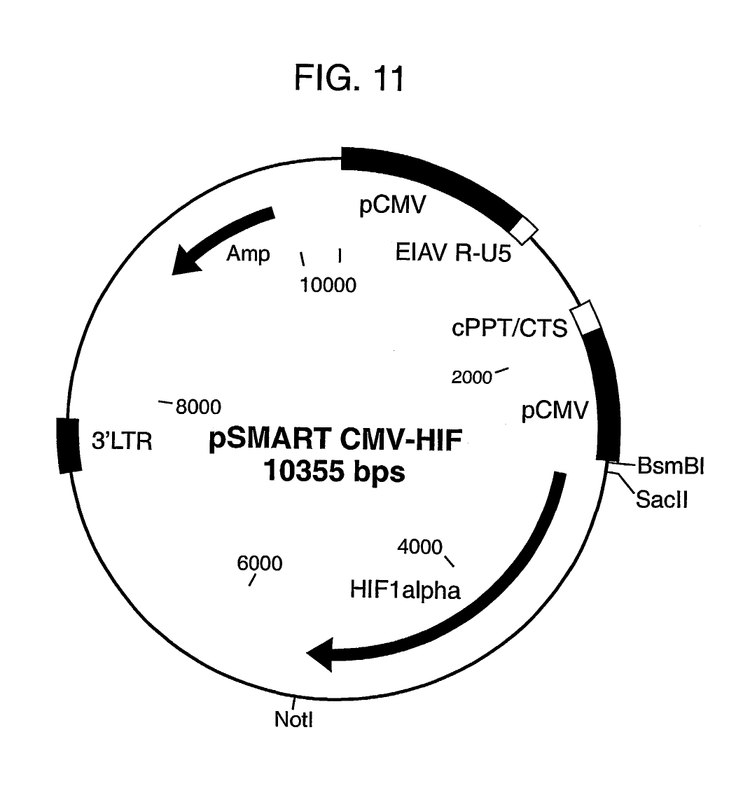

This example describes the generation of an EIAV-derived vector, pSMART CMV-HIF in which expression of HIF-1α is driven from a CMV promoter located internally within the vector (FIG. 11). A similar vector backbone could be used to achieve expression of other genes for the purposes of differential screening as described in this patent.

The starting point for construction of pSMART CMV-HIF was pONY4.0Z (WO 99/32646) and Mitophanous et al., Gene Ther. 1999 November;6(11):1808-18. In the first step, plasmid pONY4.0Z was converted into pONY8.0Z (see Example 3 above) by introducing mutations which 1) prevented expression of TAT by creating an 83 nt deletion in exon 2 of tat, 2) prevented S2 ORF expression by a 51 nt deletion, 3) prevented REV expression by deletion of a single base within exon 1 of rev, and 4) prevented expression of the N-terminal portion of gag by insertion of T residues within the first and second ATG codons of the gag region, thereby changing the sequence to ATTG from ATG. With respect to the wild type EIAV sequence (Accession No. U01866) these correspond to deletion of 1) nt 5234-5316 inclusive, 2) nt 5346-5396 inclusive, and 3) nt 5538. The insertion of T residues (4)) was after nt 526 and nt 543. These alterations were carried out using techniques readily practicable to one skilled in the art. The resulting vector, pONY8.0Z expresses none of the EIAV accessory proteins or any of the EIAV gag protein.

In the next step, the β-galactosidase reporter gene present in pONY8.0Z was replaced by the enhanced green fluorescence protein (eGFP) reporter gene to create pONY8G. This was done by transferring the SacII-KpnI fragment corresponding to the GFP gene and flanking sequences from pONY2.13GFP (WO 99/32646) into pONY8.0Z cut with the same enzymes.

The presence of sequences termed the central polypurine tract and central termination sequence (cPPT/CTS) has been suggested to improve the efficiency of gene delivery by HIV-1 based vectors to non-dividing cells (Zennou et al., Cell. April 14, 2000; 101(2):173-85, Follenzi et al., Nat Genet. 2000 June;25(2):217-22). The analogous cis-acting element of EIAV is located in the polymerase coding region and can be obtained as a functional element by using PCR amplification from any plasmid which contains the EIAV polymerase coding region (for example pONY3.1, WO 99/32646) as follows. The PCR product includes the central polypurine tract and the central termination sequence (CTS). The oligonucleotide primers used in the PCR reaction were:

EIAV cPPT POS:
CAGGTTATTCTAGAGTCGACGCTCTCATTACTTGTAAC
EIAV cPPT NEG:
CGAATGCGTTCTAGAGTCGACCATGTTCACCAGGGATTTTG

The recognition sequence for XbaI is shown in bold face and allows insertion into the pONY8G backbone-. Before insertion of the cPPT/CTS PCR product prepared as described above, pONY8G was modified to remove the central termination sequence (CTS) which was already present in the pONY8G vector. This was achieved by subcloning the SalI to ScaI fragment encompassing the CTS and RRE region from pONY8.0Z into pSP72, prepared for ligation by digestion with SalI and EcoRV. The CTS region was then excised by digestion with KpnI and PpuMI, the overhanging ends ‘blunted’ by T4 DNA polymerase treatment and then the ends religated. The modified EIAV vector fragment was then excised using SalI and NheI and ligated into pONY8G prepared for ligation by digestion with the same enzymes. This new EIAV vector was termed pONY8G del CTS. pONY8G del CTS has two XbaI sites which flank the CMV-GFP cassette and the PCR product representing the cPPT/CTS, after digestion with XbaI can be ligated into either site after partial digestion. Ligation into these sites results in plasmids with the cPPT/CTS element in either the positive or negative senses. Clones in which the cPPT/CTS was in the positive sense (functionally active) at either the 5′ or 3′-position were termed pONY8G 5′POS del CTS and pONY8G 3′POS del CTS, respectively. Another vector, termed pONY8Z 5′POS del CTS was also made following a similar strategy to that used to make pQNY8G 5′POS del CTS. Accordingly, the CTS sequence present in pONY8.0Z was removed in the same way to make pONY8Z del CTS and the cPPT/CTS sequence was introduced into the unique XbaI site just upstream of the CMV promoter in pONY8Z del CTS.

The pSMART CMV-HIF vector plasmid was derived from pONY8G 5′POS del CTS by replacement of the coding region for eGFP with that of HIF-1α. This was achieved by digestion of the latter with SacII and NotI, which flank the eGFP gene, and ligation to a SacII-NotI fragment obtained from plasmid AdApt HIF-1α-ires-GFP. Construction of plasmid AdApt HIF-1α-ires-GFP is as described in Example 2 above.

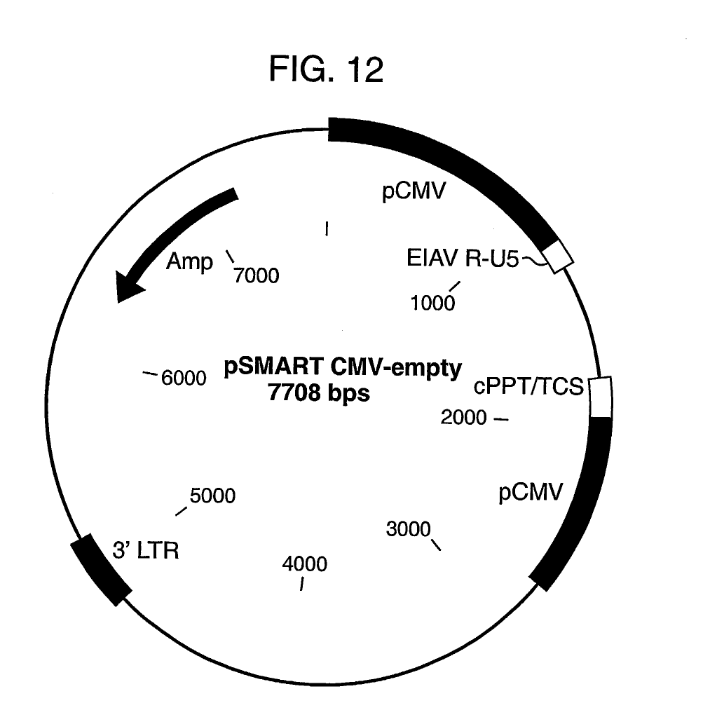

An additional derivative of pONY8G 5′POS del CTS was also made in order to produce vector preparations which serve as ‘negative controls’ in transduction experiments. This vector termed, pSMART CMV-empty (FIG. 12) was made by digestion of pONY8G 5′POS del CTS with BsmBI and NotI, which flank the eGFP gene, followed by religation. On the basis of sequence analysis of the transcript driven by the internal promoter, only a 3 amino acid peptide is expected to be produced in cells transduced with this vector.

The EIAV vectors described above were produced by transient co-transfection of 293T human embryonic kidney cells with either vector plasmid, pONY3.1 (which expresses the EIAV gag/pol protein) and an envelope expression plasmid, pRV67 (which encodes the vesicular stomatitis virus protein G, VSV-G) using the calcium phosphate precipitation method.

Twenty four hours before transfection the 293T cells were seeded at 3.6×10⁶ cells per 10 cm dish in 10 ml of DMEM supplemented with glutamine, non-essential amino acids and 10% foetal calf serum. Transfections were carried out in the late afternoon and the cells were incubated overnight prior to replacement of the medium with 6 ml of fresh media supplemented with sodium butyrate (5 mM). After 7 hours the medium was collected and 6 ml of fresh unsupplemented media added to the cells. The collected medium was cleared by low speed centrifugation and then filtered through 0.4 micron filters.

Vector particles were then concentrated by low speed centrifugation (6,000 g, JLA10.500 rotor) overnight at 4° C. and the supernatant poured off, leaving the pellet in the bottom of the tube. The following morning the remaining tissue culture fluid was harvested, cleared and filtered. It was then placed on top of the pellet previously collected and overnight centrifugation repeated. After this the supernatant was decanted and excess fluid was drained. Then the pellet was resuspended in formulation buffer to 1/1000 of the volume of starting supernatant. Aliquots were then stored at −80° C.

Formulation buffer (100 ml)

| Tissue culture grade water | | 28.65 | ml |
|---|---|---|---|
| 19.75 | mM Tris/HCl buffer pH 7.0 | 19.75 | ml of a |
| | | 0.1 | M solution |
| 40 | mg/ml lactose | 26.6 | ml of a |
| | | 150 | mg/ml solution |
| 37.5 | mM sodium chloride | 24.4 | ml of a |
| | | 154 | mM solution |
| 1 | mg/ml human serum albumin^{a} | 500 | μl of a |
| | | 20% | solution |
| 5 | μl/ml protamine sulphate^{b} | 100 | μl of a |
| | | 5 | mg/ml solution |

^{a}Human serum albumin (20%) (Albutein, Alpha therapeutics UK Ltd, Thetford, Norfolk).
^{b}Protamine sulphate 5 mg/ml (Prosulf, CP Pharmaceuticals, Wrexham, UK).

The sequence of pSMART CMV-HIF is presented in SEQ ID NO: 4.

The sequence of pSMART CMV-empty is presented in SEQ ID NO: 5.

### Example 6

Use of Smartomics for Gene Identification in Hippocampal Neurones

As discussed above in Examples 1 and 2, hypoxia is an important component of stroke (cerebral ischaemia). The present invention (Smartomics) has now been utilised to improve the discovery of genes activated or repressed in response to hypoxia in primary rat hippocampal neurones. This involves augmenting the natural response to hypoxia, by experimentally introducing a key regulator of the hypoxia response, namely hypoxia inducible factor 1α (HIF-1α). The overexpression of HIF-1α in combination with exposure of the cells to hypoxia has allowed the detection of gene expression changes which would not been detectable in response to overexpression of HIF-1α alone, or hypoxia alone.

Primary rat hippocampal neuron cultures were established according to standard procedures from embryonic rats (Dunnett S B, Bjorkland A (Eds.) 1992. Neural Transplantation, A Practical Approach. IRL Press). Briefly, timed-pregnant Wistar rats at eighteen days of gestation were anaesthetised with 0.7 ml isofluorane and killed by cervical dislocation. Pups were removed from the uterus and decapitated. Hippocampi were dissected and stored on ice in Hanks Buffered Saline Solution (HBSS) containing DNAse (0.05%) and glucose (2 mM) before incubation in trypsin (0.1%) plus DNAse (0.05%) for 5 minutes. After incubation, trypsin was inactivated by the addition of soybean trypsin inhibitor (SBTI, 0.1 %) and the solution gently triturated. Cells were pelleted by centrifugation (3000 rpm, 5 minutes) and the trypsin removed. Cells were then washed twice in HBSS containing SBTI and DNAse (0.05%), and re-pelleted before final suspension in Dulbecco's Modified Eagle's Medium (DMEM) containing foetal calf serum (10%), glutamine (2 mM), and gentamicin (0.1 mg.ml⁻¹). Cells (3×10⁶ cells per dish) were plated out onto 60 mm dishes coated with poly-D-Lysine (50 μg.ml⁻¹) and fibronectin adhesion promoting peptide (10 μg.ml⁻¹). Cultures were placed into a humidified 37° C. incubator containing 5% CO₂ and twelve hours after plating, 50% of the plating medium was replaced with Neurobasal Media (Brewer G J, (1995) “Serum-free B27/neurobasal medium supports differentiated growth of neurons from the striatum, substantia nigra, septum, cerebral cortex, cerebellum, and dentate gyrus”, Journal of Neuroscience Research 42:674-83) supplemented with B27 and glutamine (2 mM). Cultures were fed every two days with supplemented neurobasal medium and were transduced on day 3 in vitro.

Transduction was carried out in supplemented neurobasal media containing polybrene (2 μg.ml-1), in 0.5 volumes of the typical culture media volume. Five hours after the onset of transduction, the media volume was increased by a factor of 2, and was replaced 12 hours later. The viruses pSMART CMV-HIF (carrying the HIF-1α gene; see Example 5), pSMART CMV-empty (an empty genome used as a control; see Example 5) and pONY8Z 5′POS del CTS (containing the β-galactosidase gene) were produced in parallel according to methods detailed above. The pONY8Z 5′POS del CTS was used to calculate viral titer in D17 cells and in hippocampal neurons. Comparison of the RNA packaging signal by quantitative RT-PCR (Taqman) of the three viral preps, allowed the biological titers of pSMART CMV-HIF and pSMART CMV-empty viruses to be estimated relative to that pONY8Z 5′POS del CTS. All transductions were done using approximately equal multiplicity of infections (MOIs) for both viruses, and the MOI used in each experiment was at least ten.

Thirty-six hours after transduction, identical culture dishes were divided into two separate incubators, one at 37° C., 5% CO2, 95% air (=Normoxia) and the other at 37° C., 5% CO2, 94.9% Nitrogen, 0.1% Oxygen (=Hypoxia). After 6 hours culture under these conditions, the dishes were removed from the incubator, placed on a chilled platform, washed in cold PBS and total RNA was extracted using RNazol B (Tel-Test, Inc; distributed by Biogenesis Ltd) following the manufacturer's instructions.

The experiment yielded four samples, differing only in their treatment with lentivirus and/or hypoxia, as shown below:

| Sample | Lentivirus | Expressed gene | Oxygen condition |
|---|---|---|---|
| 1 | pSMART CMV-empty | none | Normoxia |
| 2 | pSMART CMV-empty | none | Hypoxia |
| 3 | pSMART CMV-HIF | HIF-1α | Normoxia |
| 4 | pSMART CMV-HIF | HIF-1α | Hypoxia |

Gene discovery can be implemented by comparing gene expression profiles between these samples. According to conventional methods published in the art, one would make comparisons between cell types 1 and 2. By implementing the present invention (Smartomics), several other possibilities are seen. Firstly, a comparison can be made between cell types 1 and 3. Here, the stimulus of overexpressing key molecules involved in the hypoxia response may exceed the natural response to hypoxia, as seen for cell type 2. Secondly, a comparison can be made between cell types 1 and 4. In this situation the natural response to hypoxia is being augmented or boosted by overexpressing key molecules involved in the hypoxia response.

Global mRNA expression profiles from the RNA isolated from the four samples were obtained using the Research Genetics Rat GeneFilter GF300 (Research Genetics, Huntsville, Ala.). This method uses pre-made nylon arrays of DNA derived from I.M.A.G.E./LLNL cDNA clones containing the 3′ ends of genes (http://image.llnl.gov/image/). The arrays include more than 5,000 genes covering a range of levels of characterisation, including sequences which are representative of unannotated ESTs or cDNA sequences of unknown function.

RNA extracted from the 4 samples described above, was radioactively labelled and hybridised to separate copies of the Research Genetics Rat GeneFilter GF300. Methods provided by the manufacturer were followed (http://www.resgen.com/products/GF200_protocol.php3) with the following modifications; RNAsin was added to the labelling reaction, and following labelling the mRNA/cDNA hybrid was denatured by incubation with 45 mM EDTA/18 mM NaOH at 65° C. for 30 minutes.

Images of hybridised arrays were obtained using a Molecular Dynamics Storm phosphorimager. RNA was then stripped from the arrays, following the aforementioned protocol. To ensure reproducibility, this procedure was repeated with the same RNA samples. Both data sets were then imported and analysed using Research Genetics Pathways 3.0 software, as explained in the Pathways 3.0 manual. Key aspects of the current analysis are summarised below:

Project Tree Set-Up

“Condition Pairs” mode was used to simultaneously analyse multiple experiments. In this context a condition is equivalent to a sample (e.g. Sample 3, overexpression of HIF-1α in normoxia).

Normalisation Set-Up

Data point normalisation was selected, as explained in the Pathways 3.0 manual. This technique generates normalised intensities by dividing all sampled intensities by the mean sampled intensity of all clones (except the control points) on the array. The two experiments were treated as separate normalisation groups, such that global differences in hybridisation signals between different arrays within the same experiment were corrected for.

Comparison Analysis

Condition 1 (i.e. Sample 1) corresponds to cells transduced with the control lentivirus and placed under normal oxygen concentrations (normoxia). This was used as the reference condition in pairwise comparisons with conditions 2, 3 and 4 (i.e. samples 2, 3 and 4). Comparisons were made in this way for all genes present on the Research Genetics GF300 array. By comparing conditions the analysis considers data from both experiments.

Results for Four Representative Known HIF-1α/Hypoxia-Regulated Genes

As demonstration that overexpression of HIF-1α in hypoxic cells is superior to using non-transduced hypoxic cells or overexpression of HIF-1α in normoxic cells, in terms of discovering bona fide hypoxia-regulated genes, results are shown below for genes which are already known in the art to be regulated by hypoxia and HIF-1α. Ratios are expressed as average ratios of normalised intensities.

TABLE 2
Response of known HIF-1α/hypoxia-regulated genes

| | | | | | RATIO SAMPLE 1 (normoxia) vs | | |
|---|---|---|---|---|---|---|---|
| | PROTEIN | | NUCLEOTIDE | | SAMPLE 2 | SAMPLE 3 | SAMPLE 4 |
| TITLE | SEQ ID | ACCESSION | SEQ ID | ACCESSION | (hypoxia) | (Hif + normoxia) | (Hif + hypoxia) |
| Enolase 1, alpha | | NP_036686 | | NM_012554 | 1.04 | 0.86 | 1.40 |
| Glucose-transporter | | AAA41248 | | M13979 | 1.41 | 0.78 | 2.14 |
| protein | | | | | | | |
| Glyceraldehyde-3- | | AAA40814 | | M29341 | 1.13 | 1.42 | 1.67 |
| phosphate dehydrogenase | | | | | | | |
| Lactate dehydrogenase A | | CAA26000 | | X01964 | 1.36 | 1.50 | 1.77 |

All four genes listed in Table 2 are known in the art to be regulated by hypoxia, and have been shown by Northern blot analysis to be down-regulated in a HIF1-α knockout (Iyer et al (1998) Cellular and developmental control of O₂ homeostasis by hypoxia-inducible factor 1α. Genes Dev 12:149-162). In the case of Enolase 1, alpha, the response to hypoxia or overexpression of Hif-1α under normoxia is undetectable by array hybridisation. It is only when Hif-1α is overexpressed under hypoxia that an increase in expression level relative to normoxia is detected. In the case of glucose-transporter protein the detectable response to hypoxia is increased by the overexpression of Hif-1α in hypoxia. In the case of both glyceraldehyde-3-phosphate dehydrogenase and Lactate dehydrogenase A the response to hypoxia is detectable, but it is increased by the overexpression of Hif-1α under normoxia, and even more so by the overexpression of Hif-1α under hypoxia.

Filter Settings

Data filtering was then performed to reduce the data set and select genes with expression ratios of above 2.0 for at least one of the three pair-wise comparisons detailed above. Genes with low signal intensities in all four conditions were automatically eliminated, using an Intensity II filter minimum of 0.2. Genes which did not respond in a reproducible way in both experiments were automatically eliminated using the Students t-test filter (90% confidence level).

Results were output as expression profiles of individual genes, showing normalised signal intensity and expression ratio. A key advantage of analysis in Pathways 3.0 is that high magnification thumbnail images of individual spots from the original images are displayed. This allows visual verification that the area being measured truly covers the region containing the hybridised array spot.

Annotation of Known and Novel Genes

As demonstration that overexpression of HIF-1α in hypoxic cells is superior to using non-transduced hypoxic cells or overexpression of HIF-1α in normoxic cells, in terms of discovering novel hypoxia-regulated genes, results are shown below for a gene which is already known in the art to be regulated by hypoxia, but not by HIF-1α, and for an unannotated gene. Ratios are expressed as average ratios of normalised intensities.

TABLE 3
Response of novel HIF-1α regulated genes

| | | | | | RATIO SAMPLE 1 (normoxia) vs | | |
|---|---|---|---|---|---|---|---|
| | PROTEIN | | NUCLEOTIDE | | SAMPLE 2 | SAMPLE 3 | SAMPLE 4 |
| TITLE | SEQ ID | ACCESSION | SEQ ID | ACCESSION | (hypoxia) | (Hif + normoxia) | (Hif + hypoxia) |
| Metallothionein-I^{a} | | AAA41590 | | J00750 | 1.61 | 1.24 | 3.49 |
| EST | | none | | AA901269 | 1.43 | 1.08 | 3.47 |

^{a}representative metallothionein ESTs are spotted twice on the array, so the data is the average of two points

Metallothionein-I is known in the literature to be regulated by hypoxia (Murphy et al (1999) Activation of metallothionein gene expression by hypoxia involves metal response elements and metal transcription factor-1. Cancer Res 59(6):1315-22), but it is not known to be regulated by HIF-1α. The data in Table 3 show that the response to overexpression of HIF- 1α in hypoxia greatly exceeds that of hypoxia alone or the overexpression of HIF-1α in normoxia. The EST (expressed sequence tag) is a completely unannotated DNA sequence. Similarly, the data in Table 3 show that the response to overexpression of HIF-1α in hypoxia greatly exceeds that of hypoxia alone or the overexpression of HIF-1α in normoxia.

This data demonstrates that the methods described above enable the further functional annotation of known genes and the functional annotation of completely unannotated novel genes with no known function.

### Example 7

The Use of Smartomics for the Identification of Genes Regulated by Cytokines

Eosinophils are associated with allergic diseases such as asthma, which is characterised by high numbers of eosinophils in affected tissue. IL-5 is a key cytokine involved in eosinophil differentiation and survival. IL-5 stimulates eosinophilopoiesis and egress from the bone marrow and also prolongs survival of peripheral blood eosinophils. As such IL-5 may play a causative role in the pathogenesis of asthma.

Genes which are activated in response to IL-5 stimulation are of interest as potential targets for asthma therapies.

A simple approach representing the state-of-the-art involves taking a population of eosinophils, dividing them in two and placing one set in the presence of IL5 and the other in the absence of IL5. RNA or protein from the two sets is then used in appropriate differential analyses. The goal would be to identify proteins or cDNAs that are present under conditions in which IL5 is present (IL5+) but not present in those cells that are maintained in medium free of IL5 (IL5−).

The present invention as applied to the identification of IL5-induced genes and proteins in eosinophils seeks to amplify the difference between IL5+ and IL5− in order to increase the signal to noise ratio. This is achieved by increasing the response to the IL5 signal by delivering the gene for an IL5 receptor to the eosinophils in a configuration where it is over-expressed.

The IL5α receptor is present in two isoforms, a membrane bound form which acts as an IL5 agonist and a soluble form which acts as an IL5 antagonist. As cells normally express both isoforms it is likely that they modulate their response in this way by maintaining a balance, between the two. Expression of one or the other should ‘force’ the eosinophil response in a way that simply altering the concentration of exogenous IL5 might not achieve.

It is expected that overexpression of the membrane bound form of the IL5α receptor would render cells hyperresponsive to the cytokine. In a differential screen, overexpression of this form of the receptor will lead to amplification of levels of IL5 specific cDNAs or proteins. The probability of detecting targets for drug development will therefore increase. The present invention as applied to this case involves comparison of eosinophils that are not overexpressing the membrane bound form of the IL5α receptor in the absence of IL5 ligand, with eosinophils exposed to IL5 and overexpressing the membrane bound form of the IL5α receptor.

Similarly, overexpression of the soluble form of the receptor, which acts as an IL-5 antagonist, would be expected to diminish the response of eosinophils to stimulation by IL-5. The expression profile of eosinophils overexpressing the soluble form of the IL5α receptor in the absence of IL5 ligand is compared to that of eosinophils exposed to IL5 (but not overexpressing soluble IL5α receptor). Either of these approaches may be used to distinguish genes which are expressed in response to IL5 and whose products are potential targets for therapy of allergic diseases such as asthma.

Any cell line which expresses IL5 receptor may be used, for example, AML14.3D10, TF-1.8 or HL-60. Delivery and expression of membrane bound and soluble forms of IL5α receptor may be achieved by a variety of ways. For example, eosinophils may be transfected or transduced with expression constructs as described in the Examples above, and Example 8 below.

Gene expression in transduced and untransduced eosinophil populations is compared in a number of ways as described below to generate read-outs of genes that are expressed in response to IL5. Cells transfected with construct expressing soluble IL5α receptor in the absence of IL5 are compared with untransfected cells in presence of IL5. Cells transfected with construct expressing membrane bound IL5α receptor in the presence of IL5 are compared with untransfected cells in absence of IL5.

Total RNA samples are prepared for the analysis of differential gene expression. These are labelled either radioactively or fluorescently, and hybridized to arrays of cDNAs on solid supports. Genes which are upregulated by IL5 produce quantitatively stronger hybridization signals. Array strategies may involve either nylon or glass supports, which are reviewed in Bowtell, 1999. Details of methodologies involved in the glass support approach are detailed in Eisen and Brown, 1999. Here fluorescently labelled probes are used and hybridization is detected using a laser confocal scanner. For the Nylon support approach, standard molecular biology methods of dot blotting and hybridization-are involved as detailed in Molecular Cloning: A laboratory manual Sambrook, J et al, Cold Spring Harbor Laboratory Press. Here, RNA samples to be compared are radioactively labelled and hybridization is detected using a phosphorimager.

Arrays can be purchased from Research Genetics, Huntsville, Ala. or would be fabricated in-house using cDNA clones generated by subtraction cloning (PCR-Select method, owned by Clontech Palo Alto, Calif.). Fabrication would involve use of an arraying robot (MicroGrid, BioRobotics Ltd, Cambridge, UK).

The RNA isolated from cells may be reverse-transcribed to cDNA and the cDNA screened accordingly. Alternatively, and as described above, a proteomics approach may be used to identify differentially expressed products, for example, by 2-D gel electrophoresis. Reference is made to Blackstock and Weir (1999) and the references cited therein, in which a variety of proteomics techniques is discussed.

The differential expression pattern of other cells which are responsive to IL5, for example, basophils and bone marrow precursors, may also be determined using the above method. Other cells which do not normally respond to IL5 may also be used, provided the β chain of the IL5 is co-expressed with theα chain. In this regard, it is to be noted that a common β chain is shared between the IL-5, IL-3 and GM-CSF receptors.

### Example 8

Overexpression of Human IL5αR Isoforms

This example describes the generation of two EIAV vectors (pONY8.1SMIL5Rm and pONY8.1SMIL5Rs) that are able to express the interleukin 5 alpha membrane receptor (pONY8.1SMIL5Rm) or the interleukin 5 alpha soluble receptor (pONY8.1SMIL5Rs) from an internal CMV promoter. The accession number for human IL5αR is A2625 1.

[Human IL5 alpha receptor gene: A26251, AUTHORS: Devos, R., Fiers, W., Plaetinck, G., Tavernier, J. and van der Heyden, TITLE: Human Interleukin-5 receptor, PATENT: EP 0492214-A 11 1 Jul. 1992; F. HOFFMANN-LA ROCHE A G]

To make pONYS8.1SMIL5Rm, the IL5αR was PCR amplified from cDNA generated from mRNA isolated from human peripheral blood eosinophils. The primers for this were IL5R1 and IL5R2 described below. They contain Sbf I sites for cloning and the Kozak sequence has been used to enhance translation. The PCR product generated this way contains Sbf I cloning sites flanking the IL5αR open reading frame. This was cut with Sbf I and inserted into pONY8.1SM cut with Sbf I. It is important to check that the IL5αR has inserted in the correct orientation. The plasmid generated this way was called pONY8.1SMIL5Rm.

This construct will express the wild type IL5αR. The IL5αR open reading frame was modified to make pONY8.1SMIL5Rs which expresses the soluble form of IL5αR.

This was done by PCR amplification to remove the C terminus of the receptor (Epitope-labelled soluble human interleukin-5 (IL-5), receptors. Affinity cross-link labeling, IL-5 binding, and biological activity. Brown P M, Tagari P, Rowan K R, Yu V L, O'Neill G P, Middaugh C R, Sanyal G, Ford-Hutchinson A W, Nicholson D W). The first 332 amino acids are retained while the last 88 amino acids comprising the transmembrane and intracellular region are removed. The primers for this were IL5R1 and IL5R3 described below. They contain Sbf I sites for cloning and the Kozak sequence has been used to enhance translation. The PCR product generated this way contains Sbf I cloning sites flanking the IL5αR open reading frame. This was cut with Sbf I and inserted into pONY8.1SM cut with Sbf I. It is important to check that the IL5αR has inserted in the correct orientation. The plasmid generated this way was called pONY8.1SMIL5Rs.

IL5R1 Primer

ATCGCCTGCAGGCCACCATGATGATCATCGTGGCGCATGTATTAC

Sbf I site=underlined

Kozak sequence=bold and italics

ATG start codon=underlined and italics

IL5R2 Primer

ACTGCCTGCAGGTCAAAACACAGAATCCTCCAGGGTC

Sbf I site=underlined

IL5R Primer

ACTGCCTGCAGGTCATCCCACATAAATAGGTTGGCTC

Sbf I site=underlined

Other Examples

Overexpressing anti-apoptotic genes (ie. Bcl-2, Bcl-x) in a dopaminegic cell line leads to neuroprotection from neurotoxins such as MPTP. As the more representative dopaminegic neurons (primary cells) are postmitotic in culture, lentiviral vectors can be used to introduce and overexpress such genes into these neurons and then screen for cellular targets that become differentially expressed.

Anti-apoptotic targets can also be identified by overexpressing (apoptotic) death receptors in neurons such as Fas and supplying ligand (FasL) in limited amounts. These cells will try to survive by inducing their neuroprotective genes.

Similarly growth factors (NGF, GDNF etc), and their receptors can be overexpressed in cell lines making the cells supersensitive to the survival effects of the growth factor.

Heat shock proteins (HSPs) such as HSP70 are expressed after stressful insults in the nervous system and their over-production leads to protection in several different models of nervous system injury. HSPs are implicated in cerebral ischemia, neurodegenerative diseases, epilepsy and trauma. HSPs are chaperones normally bound to heat shock factors (HSFs) which after injury become dissociated in the cytosol, phosporylated and trimerised and enter the nucleus where they bind to heat shock elements (HSEs) within the promoter of heat shock genes leading to their transcriptional activation. Therefore overexpression of HSPs in neurons, glia or endothelial cells can be used for differential screening in a similar manner to that of Hif1.

APP (amyloid precursor protein): a trans-membrane protein which is the precursor of the Aβ peptide which is found in neuritic plaques in Alzheimer's disease. Mutations have been identified which are causative of the some of the familial (early onset) forms of the disease.

Presenilins 1 and 2: trans-membrane proteins central to the processing of APP and some other membrane proteins. Several mutations have been isolated in some of the familial forms of the disease.

α-synuclein: A cytoplasmic protein associated with neuronal synapses. Mutations have been found in few Parkinson's pedigrees. Part of Lewy body (intracellular lesions characteristic of Parkinson's disease and also found in Alzheimers disease and Lewy body dementia).

Tau: a microtubule binding protein. Mutations have been found in frontal temporal dementia with Parkinsonism linked to chromosome 17 and Pick's disease.

Parkin: protein of unknown function with some homology to ubiquitin at the N-terminus and a RING-finger motif at the C-terminus. Deletions identified in juvenile form of Parkinson's disease.

Ubiquitin (UCH-L1): a thiol protease that forms part of the Lewy body. Mutations have been identified in a German Parkinson's disease pedigree.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

References

Blackstock and Weir (1999) Trends in Biotech. 17: 121-126.

Bowtell (1999) Nature Genetics 21: 25-32.

Eisen and Brown (1-999). Methods Enymol. 303: 179-205.

Griffiths L. et al. (2000), Gene Therapy 7: 255-262.

Jorgensen et al. (1999), Electrophoresis 2: 230-40.

Kirschbaum and Kozian (1999), Trends in Biotech 17: 73-78.

Mitophanous et al. (1999), Gene Therapy 6: 1808-1818.

Pardee and Liang (1992), Science 257: 967-971.

Rabilloud et al. (1997), Electrophoresis 18: 307-316.

Soneoka et al. (1995), Nucleic Acids Res. 23: 628-33.

Wilkinson et al. (1995), Plant Mol Biol 6: 1097-108.

Zhang et al. (1998), Mol Biotechnol 10(2): 155-65.

Zhao et al. (1999), J Biotechnol. 73(1): 35-41.

Nucleotide sequence of ires-GFP DNA fragment
SEQ ID NO:1
CTAGAGTGTGATTTTAAGGGCGAATTCTGCAGATATCCATCACACTGGCGGCCGCACTAGAGGAATTCGCC
CCTCTCCCTCCCCCCCCCCTAACGTTACTGGCCGAAGCCGCTTGGAATAAGGCCGGTGTGTGTTTGTCTAT
ATGTGATTTTCCACCATATTGCCGTCTTTTGGCAATGTGAGGGCCCGGAAACCTGGCCCTGTCTTCTTGAC
GAGCATTCCTAGGGGTCTTTCCCCTCTCGCCAAAGGAATGCAAGGTCTGTTGAATGTCGTGAAGGAAGCAG
TTCCTCTGGAAGCTTCTTGAAGACAAACAACGTCTGTAGCGACCCTTTGCAGGCAGCGGAACCCCCCACCT
GGCGACAGGTGCCTCTGCGGCCAAAAGCCACGTGTATAAGATACACCTGCAAAGGCGGCACAACCCCAGTG
CCACGTTGTGAGTTGGATAGTTGTGGAAAGAGTCAAATGGCTCTCCTCAAGCGTAGTCAACAAGGGGCTGA
AGGATGCCCAGAAGGTACCCCATTGTATGGGAATCTGATCTGGGGCCTCGGTGCACATGCTTTACATGTGT
TTAGTCGAGGTTAAAAAAGCTCTAGGCCCCCCGAACCACGGGGACGTGGTTTTCCTTTGAAAAACACGATG
ATACCATGGTGAGCAAGGGCGAGGAGCTGTTCACCGGGGTGGTGCCCATCCTGGTCGAGCTGGACGGCGAC
GTAAACGGCCACAAGTTCAGCGTGTCCGGCGAGGGCGAGGGCGATGCCACCTACGGCAAGCTGACCCTGAA
GTTCATCTGCACCACCGGCAAGCTGCCCGTGCCCTGGCCCACCCTCGTGACCACCCTGACCTACGGCGTGC
AGTGCTTCAGCCGCTACCCCGACCACATGAAGCAGCACGACTTCTTCAAGTCCGCCATGCCCGAAGGCTAC
GTCCAGGAGCGCACCATCTTCTTCAAGGACGACGGCAACTACAAGACCCGCGCCGAGGTGAAGTTCGAGGG
CGACACCCTGGTGAACCGCATCGAGCTGAAGGGCATCGACTTCAAGGAGGACGGCAACATCCTGGGGCACA
AGCTGGAGTACAACTACAACAGCCACAACGTCTATATCATGGCCGACAAGCAGAAGAACGGCATCAAGGTG
AACTTCAAGATCCGCCACAACATCGAGGACGGCAGCGTGCAGCTCGCCGACCACTACCAGCAGAACACCCC
CATCGGCGACGGCCCCGTGCTGCTGCCCGACAACCACTACCTGAGCACCCAGTCCGCCCTGAGCAAAGACC
CCAACGAGAAGCGCGATCACATGGTCCTGCTGGAGTTCGTGACCGCCGCCGGGATCACTCTCGGCATGGAC
GAGCTGTACAAGTAAAGCGGCCGCGACT

Nucleotide sequence of DNA fragment containing human HIF-α protein coding sequence
SEQ ID NO:2
CTAGCCGTAGAATCCGACCGATTCACCATGGAGGGCGCCGGCGGCGCGAACGACAAGAAAAAGATAAGTTCTGAACGTCGAA
AAGAAAAGTCTCGAGATGCAGCCAGATCTCGGCGAAGTAAAGAATCTGAAGTTTTTTATGAGCTTGCTCATCAGTTGCCACT
TCCACATAATGTGAGTTCGCATCTTGATAAGGCCTCTGTGATGAGGCTTACCATCAGCTATTTGCGTGTGAGGAAACTTCTG
GATGCTGGTGATTTGGATATTGAAGATGACATGAAAGCACAGATGAATTGCTTTTATTTGAAAGCCTTGGATGGTTTTGTTA
TGGTTCTCACAGATGATGGTGACATGATTTACATTTCTGATAATGTGAACAAATACATGGGATTAACTCAGTTTGAACTAAC
TGGACACAGTGTGTTTGATTTTACTCATCCATGTGACCATGAGGAAATGAGAGAAATGCTTACACACAGAAATGGCCTTGTG
AAAAAGGGTAAAGAACAAAACACACAGCGAAGCTTTTTTCTCAGAATGAAGTGTACCCTAACTAGCCGAGGAAGAACTATGA
ACATAAAGTCTGCAACATGGAAGGTATTGCACTGCACAGGCCACATTCACGTATATGATACCAACAGTAACCAACCTCAGTG
TGGGTATAAGAAACCACCTATGACCTGCTTGGTGCTGATTTGTGAACCCATTCCTCACCCATCAAATATTGAAATTCCTTTA
GATAGCAAGACTTTCCTCAGTCGACACAGCCTGGATATGAAATTTTCTTATTGTGATGAAAGAATTACCGAATTGATGGGAT
ATGAGCCAGAAGAACTTTTAGGCCGCTCAATTTATGAATATTATCATGCTTTGGACTCTGATCATCTGACCAAAACTCATCA
TGATATGTTTACTAAAGGACAAGTCACCACAGGACAGTACAGGATGCTTGCCAAAAGAGGTGGATATGTCTGGGTTGAAACT
CAAGCAACTGTCATATATAACACCAAGAATTCTCAACCACAGTGCATTGTATGTGTGAATTACGTTGTGAGTGGTATTATTC
AGCACGACTTGATTTTCTCCCTTCAACAAACAGAATGTGTCCTTAAACCGGTTGAATCTTCAGATATGAAAATGACTCAGCT
ATTCACCAAAGTTGAATCAGAAGATACAAGTAGCCTCTTTGACAAACTTAAGAAGGAACCTGATGCTTTAACTTTGCTGGCC
CCAGCCGCTGGAGACACAATCATATCTTTAGATTTTGGCAGCAACGACACAGAAACTGATGACCAGCAACTTGAGGAAGTAC
CATTATATAATGATGTAATGCTCCCCCTCACCAACGAAAAATTACAGAATATAAATTTGGCAATGTCTCCATTACCCACCGC
TGAAACGCCAAAGCCACTTCGAAGTAGTGCTGACCCTGCACTCAATCAAGAAGTTGCATTAAAATTAGAACCAAATCCAGAG
TCACTGGAACTTTCTTTTACCATGCCCCAGATTCAGGATCAGACACCTAGTCCTTCCGATGGAAGCACTAGACAAAGTTCAC
CTGAGCCTAATAGTCCCAGTGAATATTGTTTTTATGTGGATAGTGATATGGTCAATGAATTCAAGTTGGAATTGGTAGAAAA
ACTTTTTGCTGAAGACACAGAAGCAAAGAACCCATTTTCTACTCAGGACACAGATTTAGACTTGGAGATGTTAGCTCCCTAT
ATCCCAATGGATGATGACTTCCAGTTACGTTCCTTCGATCAGTTGTCACCATTAGAAAGCAGTTCCGCAAGCCCTGAAAGCG
CAAGTCCTCAAAGCACAGTTACAGTATTCCAGCAGACTCAAATACAAGAACCTACTGCTAATGCCACCACTACCACTGCCAC
CACTGATGAATTAAAAACAGTGACAAAAGACCGTATGGAAGACATTAAAATATTGATTGCATCTCCATCTCCTACCCACATA
CATAAAGAAACTACTAGTGCCACATCATCACCATATAGAGATACTCAAAGTCGGACAGCCTCACCAAACAGAGCAGGAAAAG
GAGTCATAGAACAGACAGAAAAATCTCATCCAACAAGCCCTAACGTGTTATCTGTCGCTTTGAGTCAAAGAACTACAGTTCC
TGAGGAAGAACTAAATCCAAACATACTAGCTTTGCAGAATGCTCAGAGAAAGCGAAAAATGGAACATGATGGTTCACTTTTT
CAAGCAGTAGGAATTGGAACATTATTACAGCAGCCAGACGATCATGCAGCTACTACATCACTTTCTTGGAAACGTGTAAAAG
GATGCAAATCTAGTGAACAGAATGGAATGGAGCAAAAGACAATTATTTTAATACCCTCTGATTTAGCATGTAGACTGCTGGG
GCAATCAATGGATGAAAGTGGATTACCACAGCTGACCAGTTATGATTGTGAAGTTAATGCTCCTATACAAGGCAGCAGAAAC
CTACTGCAGGGTGAAGAATTACTCAGAGCTTTGGATCAAGTTAACTGAGCGGATCCGACGGGGATCCT

Nucleotide sequence of DNA fragment containing human EPAS1 protein coding sequence
SEQ ID NO:3
AGCTTGCATGCCTGCAGGTCGACTCTAGAGGATCCAGCGACAATGACAGCTGACAAGGAGAAGAAAAGGAGTAGCTCGGAGA
GGAGGAAGGAGAAGTCCCGGGATGCTGCGCGGTGCCGGCGGAGCAAGGAGACGGAGGTGTTCTATGAGCTGGCCCATGAGCT
GCCTCTGCCCCACAGTGTGAGCTCCCATCTGGACAAGGCCTCCATCATGCGACTGGAAATCAGCTTCCTGCGAACACACAAG
CTCCTCTCCTCAGTTTGCTCTGAAAACGAGTCCGAAGCCGAAGCTGACCAGCAGATGGACAACTTGTACCTGAAAGCCTTGG
AGGGTTTCATTGCCGTGGTGACCCAAGATGGCGACATGATCTTTCTGTCAGAAAACATCAGCAAGTTCATGGGACTTACACA
GGTGGAGCTAACAGGACATAGTATCTTTGACTTCACTCATCCCTGCGACCATGAGGAGATTCGTGAGAACCTGAGTCTCAAA
AATGGCTCTGGTTTTGGGAAAAAAAGCAAAGACATGTCCACAGAGCCGGACTTCTTCATGAGGATGAAGTGCACGGTCACCA
ACAGAGCCCGTACTGTCAACCTCAAGTCAGCCACCTGGAAGGTCTTGCACTGCACGGGCCAGGTGAAAGTCTACAACAACTG
CCCTCCTCACAATAGTCTGTGTGGCTACAAGGAGCCCCTGCTGTCCTGCCTCATCATCATGTGTGAACCAATCCAGCACCCA
TCCCACATGGACATCCCCCTGGATAGCAAGACCTTCCTGAGCCGCCACAGCATGGACATGAAGTTCACCTACTGTGATGACA
GAATCACAGAACTGATTGGTTACCACCCTGAGGAGCTGCTTGGCCGCTCAGCCTATGAATTCTACCATGCGCTAGACTCCGA
GAACATGACCAAGAGTCACCAGAACTTGTGCACCAAGGGTCAGGTAGTAAGTGGCCAGTACCGGATGCTCGCAAAGCATGGG
GGCTACGTGTGGCTGGAGACCCAGGGGACGGTCATCTACAACCCTCGCAACCTGCAGCCCCAGTGCATCATGTGTGTCAACT
ACGTCCTGAGTGAGATTGAGAAGAATGACGTGGTGTTCTCCATGGACCAGACTGAATCCCTGTTCAAGCCCCACCTGATGGC
CATGAACAGCATCTTTGATAGCAGTGGCAAGGGGGCTGTGTCTGAGAAGAGTAACTTCCTATTCACCAAGCTAAAGGAGGAG
CCCGAGGAGCTGGCCCAGCTGGCTCCCACCCCAGGAGACGCCATCATCTCTCTGGATTTCGGGAATCAGAACTTCGAGGAGT
CCTCAGCCTATGGCAAGGCCATCCTGCCCCCGAGCCAGCCATGGGCCACGGAGTTGAGGAGCCACAGCACCCAGAGCGAGGC
TGGGAGCCTGCCTGCCTTCACCGTGCCCCAGGCAGCTGCCCCGGGCAGCACCACCCCCAGTGCCACCAGCAGCAGCAGCAGC
TGCTCCACGCCCAATAGCCCTGAAGACTATTACACATCTTTGGATAACGACCTGAAGATTGAAGTGATTGAGAAGCTCTTCG
CCATGGACACAGAGGCCAAGGACCAATGCAGTACCCAGACGGATTTCAATGAGCTGGACTTGGAGACACTGGCACCCTATAT
CCCCATGGACGQGGAAGACTTCCAGCTAAGCCCCATCTGCCCCGAGGAGCGGCTCTTGGCGGAGAACCCACAGTCCACCCCC
CAGCACTGCTTCAGTGCCATGACAAACATCTTCCAGCCACTGGCCCCTGTAGCCCCGCACAGTCCCTTCCTCCTGGACAAGT
TTCAGCAGCAGCTGGAGAGCAAGAAGACAGAGCCCGAGCACCGGCCCATGTCCTCCATCTTCTTTGATGCCGGAAGCAAAGC
ATCCCTGCCACCGTGCTGTGGCCAGGCCAGCACCCCTCTCTCTTCCATGGGGGGCAGATCCAATACCCAGTGGCCCCCAGAT
CCACCATTACATTTTGGGCCCACAAAGTGGGCCGTCGGGGATCAGCGCACAGAGTTCTTGGGAGCAGCGCCGTTGGGGCCCC
CTGTCTCTCCACCCCATGTCTCCACCTTCAAGACAAGGTCTGCAAAGGGTTTTGGGGCTCGAGGCCCAGACGTGCTGAGTCC
GGCCATGGTAGCCCTCTCCAACAAGCTGAAGCTGAAGCGACAGCTGGAGTATGAAGAGCAAGCCTTCCAGGACCTGAGCGGG
GGGGACCCACCTGGTGGCAGCACCTCACATTTGATGTGGAAACGGATGAAGAACCTCAGGGGTGGGAGCTGCCCTTTGATGC
CGGACAAGCCACTGAGCGCAAATGTACCCAATGATAAGTTCACCCAAAACCCCATGAGGGGCCTGGGCCATCCCCTGAGACA
TCTGCCGCTGCCACAGCCTCCATCTGCCATCAGTCCCGGGGAGAACAGCAAGAGCAGGTTCCCCCCACAGTGCTACGCCACC
CAGTACCAGGACTACAGCCTGTCGTCAGCCCACAAGGTGTCAGGCATGGCAAGCCGGCTGCTCGGGCCCTCATTTGAGTCCT
ACCTGCTGCCCGAACTGACCAGATATGACTGTGAGGTGAACGTGCCCGTGCTGGGAAGCTCCACGCTCCTGCAAGGAGGGGA
CCTCCTCAGAGCCCTGGACCAGGCCACCTGAGCCAGGCCTTCTACCTGGGCAGCACCTCTGCCCACGCCGAGCCCTATGCAG
TCTCGGCCGCAAGCTATCAGATCTGCCGGTCTCCCTATAGTGAGTCGTATTAATTTCGATAAGCCAGGTT

| The nucleotide sequence of pSMART CMV-HIF | | |
|---|---|---|
| 1 | AGATCTTGAA TAATAAAATG TGTGTTTGTC CGAAATACGC GTTTTGAGAT | SEQ ID NO:4 |
| 51 | TTCTGTCGCC GACTAAATTC ATGTCGCGCG ATAGTGGTGT TTATCGCCGA | |
| 101 | TAGAGATGGC GATATTGGAA AAATTGATAT TTGAAAATAT GGCATATTGA | |
| 151 | AAATGTCGCC GATGTGAGTT TCTGTGTAAC TGATATCGCC ATTTTTCCAA | |
| 201 | AAGTGATTTT TGGGCATACG CGATATCTGG CGATAGCGCT TATATCGTTT | |
| 251 | ACGGGGGATG GCGATAGACG ACTTTGGTGA CTTGGGCGAT TCTGTGTGTC | |
| 301 | GCAAATATCG CAGTTTCGAT ATAGGTGACA GACGATATGA GGCTATATCG | |
| 351 | CCGATAGAGG CGACATCAAG CTGGCACATG GCCAATGCAT ATCGATCTAT | |
| 401 | ACATTGAATC AATATTGGCC ATTAGCCATA TTATTCATTG GTTATATAGC | |
| 451 | ATAAATCAAT ATTGGCTATT GGCCATTGCA TACGTTGTAT CCATATCGTA | |
| 501 | ATATGTACAT TTATATTGGC TCATGTCCAA CATTACCGCC ATGTTGACAT | |
| 551 | TGATTATTGA CTAGTTATTA ATAGTAATCA ATTACGGGGT CATTAGTTCA | |
| 601 | TAGCCCATAT ATGGAGTTCC GCGTTACATA ACTTACGGTA AATGGCCCGC | |
| 651 | CTGGCTGACC GCCCAACGAC CCCCGCCCAT TGACGTCAAT AATGACGTAT | |
| 701 | GTTCCCATAG TAACGCCAAT AGGGACTTTC CATTGACGTC AATGGGTGGA | |
| 751 | GTATTTACGG TAAACTGCCC ACTTGGCAGT ACATCAAGTG TATCATATGC | |
| 801 | CAAGTCCGCC CCCTATTGAC GTCAATGACG GTAAATGGCC CGCCTGGCAT | |
| 851 | TATGCCCAGT ACATGACCTT ACGGGACTTT CCTACTTGGC AGTACATCTA | |
| 901 | CGTATTAGTC ATCGCTATTA CCATGGTGAT GCGGTTTTGG CAGTACACCA | |
| 951 | ATGGGCGTGG ATAGCGGTTT GACTCACGGG GATTTCCAAG TCTCCACCCC | |
| 1001 | ATTGACGTCA ATGGGAGTTT GTTTTGGCAC CAAAATCAAC GGGACTTTCC | |
| 1051 | AAAATGTCGT AACAACTGCG ATCGCCCGCC CCGTTGACGC AAATGGGCGG | |
| 1101 | TAGGCGTGTA CGGTGGGAGG TCTATATAAG CAGAGCTCGT TTAGTGAACC | |
| 1151 | GGGCACTCAG ATTCTGCGGT CTGAGTCCCT TCTCTGCTGG GCTGAAAAGG | |
| 1201 | CCTTTGTAAT AAATATAATT CTCTACTCAG TCCCTGTCTC TAGTTTGTCT | |
| 1251 | GTTCGAGATC CTACAGTTGG CGCCCGAACA GGGACCTGAG AGGGGCGCAG | |
| 1301 | ACCCTACCTG TTGAACCTGG CTGATCGTAG GATCCCCGGG ACAGCAGAGG | |
| 1351 | AGAACTTACA GAAGTCTTCT GGAGGTGTTC CTGGCCAGAA CACAGGAGGA | |
| 1401 | CAGGTAAGAT TGGGAGACCC TTTGACATTG GAGCAAGGCG CTCAAGAAGT | |
| 1451 | TAGAGAAGGT GACGGTACAA GGGTCTCAGA AATTAACTAC TGGTAACTGT | |
| 1501 | AATTGGGCGC TAAGTCTAGT AGACTTATTT CATGATACCA ACTTTGTAAA | |
| 1551 | AGAAAAGGAC TGGCAGCTGA GGGATGTCAT TCCATTGCTG GAAGATGTAA | |
| 1601 | CTCAGACGCT GTCAGGACAA GAAAGAGAGG CCTTTGAAAG AACATGGTGG | |
| 1651 | GCAATTTCTG CTGTAAAGAT GGGCCTCCAG ATTAATAATG TAGTAGATGG | |
| 1701 | AAAGGCATCA TTCCAGCTCC TAAGAGCGAA ATATGAAAAG AAGACTGCTA | |
| 1751 | ATAAAAAGCA GTCTGAGCCC TCTGAAGAAT ATCTCTAGAG TCGACGCTCT | |
| 1801 | CATTACTTGT AACAAAGGGA GGGAAAGTAT GGGAGGACAG ACACCATGGG | |
| 1851 | AAGTATTTAT CACTAATCAA GCACAAGTAA TACATGAGAA ACTTTTACTA | |
| 1901 | CAGCAAGCAC AATCCTCCAA AAAATTTTGT TTTTACAAAA TCCCTGGTGA | |
| 1951 | ACATGGTCGA CTCTAGAACT AGTGGATCCC CCGGGCTGCA GGAGTGGGGA | |
| 2001 | GGCACGATGG CCGCTTTGGT CGAGGCGGAT CCGGCCATTA GCCATATTAT | |
| 2051 | TCATTGGTTA TATAGCATAA ATCAATATTG GCTATTGGCC ATTGCATACG | |
| 2101 | TTGTATCCAT ATCATAATAT GTACATTTAT ATTGGCTCAT GTCCAACATT | |
| 2151 | ACCGCCATGT TGACATTGAT TATTGACTAG TTATTAATAG TAATCAATTA | |
| 2201 | CGGGGTCATT AGTTCATAGC CCATATATGG AGTTCCGCGT TACATAACTT | |
| 2251 | ACGGTAAATG GCCCGCCTGG CTGACCGCCC AACGACCCCC GCCCATTGAC | |
| 2301 | GTCAATAATG ACGTATGTTC CCATAGTAAC GCCAATAGGG ACTTTCCATT | |
| 2351 | GACGTCAATG GGTGGAGTAT TTACGGTAAA CTGCCCACTT GGCAGTACAT | |
| 2401 | CAAGTGTATC ATATGCCAAG TACGCCCCCT ATTGACGTCA ATGACGGTAA | |
| 2451 | ATGGCCCGCC TGGCATTATG CCCAGTACAT GACCTTATGG GACTTTCCTA | |
| 2501 | CTTGGCAGTA CATCTACGTA TTAGTCATCG CTATTACCAT GGTGATGCGG | |
| 2551 | TTTTGGCAGT ACATCAATGG GCGTGGATAG CGGTTTGACT CACGGGGATT | |
| 2601 | TCCAAGTCTC CACCCCATTG ACGTCAATGG GAGTTTGTTT TGGCACCAAA | |
| 2651 | ATCAACGGGA CTTTCCAAAA TGTCGTAACA ACTCCGCCCC ATTGACGCAA | |
| 2701 | ATGGGCGGTA GGCATGTACG GTGGGAGGTC TATATAAGCA GAGCTCGTTT | |
| 2751 | AGTGAACCGT CAGATCGCCT GGAGACGCCA TCCACGCTGT TTTGACCTCC | |
| 2801 | ATAGAAGACA CCGGGACCGA TCCAGCCTCC GCGGCCGGGA ACGGTGCATT | |
| 2851 | GGAAGCTTGG TACCGGCTAG CCGTAGAATC CGACCGATTC ACCATGGAGG | |
| 2901 | GCGCCGGCGG CGCGAACGAC AAGAAAAAGA TAAGTTCTGA ACGTCGAAAA | |
| 2951 | GAAAAGTCTC GAGATGCAGC CAGATCTCGG CGAAGTAAAG AATCTGAAGT | |
| 3001 | TTTTTATGAG CTTGCTCATC AGTTGCCACT TCCACATAAT GTGAGTTCGC | |
| 3051 | ATCTTGATAA GGCCTCTGTG ATGAGGCTTA CCATCAGCTA TTTGCGTGTG | |
| 3101 | AGGAAACTTC TGGATGCTGG TGATTTGGAT ATTGAAGATG ACATGAAAGC | |
| 3151 | ACAGATGAAT TGCTTTTATT TGAAAGCCTT GGATGGTTTT GTTATGGTTC | |
| 3201 | TCACAGATGA TGGTGACATG ATTTACATTT CTGATAATGT GAACAAATAC | |
| 3251 | ATGGGATTAA CTCAGTTTGA ACTAACTGGA CACAGTGTGT TTGATTTTAC | |
| 3301 | TCATCCATGT GACCATGAGG AAATGAGAGA AATGCTTACA CACAGAAATG | |
| 3351 | GCCTTGTGAA AAAGGGTAAA GAACAAAACA CACAGCGAAG CTTTTTTCTC | |
| 3401 | AGAATGAAGT GTACCCTAAC TAGCCGAGGA AGAACTATGA ACATAAAGTC | |
| 3451 | TGCAACATGG AAGGTATTGC ACTGCACAGG CCACATTCAC GTATATGATA | |
| 3501 | CCAACAGTAA CCAACCTCAG TGTCGGTATA AGAAACCACC TATGACCTGC | |
| 3551 | TTGGTGCTGA TTTGTGAACC CATTCCTCAC CCATCAAATA TTGAAATTCC | |
| 3601 | TTTAGATAGC AAGACTTTCC TCAGTCGACA CAGCCTGGAT ATGAAATTTT | |
| 3651 | CTTATTGTGA TGAAAGAATT ACCGAATTGA TGGGATATGA GCCAGAAGAA | |
| 3701 | CTTTTAGGCC GCTCAATTTA TGAATATTAT CATGCTTTGG ACTCTGATCA | |
| 3751 | TCTGACCAAA ACTCATCATG ATATGTTTAC TAAAGGACAA GTCACCACAG | |
| 3801 | GACAGTACAG GATGCTTGCC AAAAGAGGTG GATATGTCTG GGTTGAAACT | |
| 3851 | CAAGCAACTG TCATATATAA CACCAAGAAT TCTCAACCAC AGTGCATTGT | |
| 3901 | ATGTGTGAAT TACGTTGTGA GTGGTATTAT TCAGCACGAC TTGATTTTCT | |
| 3951 | CCCTTCAACA AACAGAATGT GTCCTTAAAC CGGTTGAATC TTCAGATATG | |
| 4001 | AAAATGACTC AGCTATTCAC CAAAGTTGAA TCAGAAGATA CAAGTAGCCT | |
| 4051 | CTTTGACAAA CTTAAGAAGG AACCTGATGC TTTAACTTTG CTGGCCCCAG | |
| 4101 | CCGCTGGACA CACAATCATA TCTTTAGATT TTGGCAGCAA CGACACAGAA | |
| 4151 | ACTGATGACC AGCAACTTGA GGAAGTACCA TTATATAATG ATGTAATGCT | |
| 4201 | CCCCTCACCC AACGAAAAAT TACAGAATAT AAATTTGGCA ATGTCTCCAT | |
| 4251 | TACCCACCGC TGAAACGCCA AAGCCACTTC GAAGTAGTGC TGACCCTGCA | |
| 4301 | CTCAATCAAG AAGTTGCATT AAAATTAGAA CCAAATCCAG AGTCACTGGA | |
| 4351 | ACTTTCTTTT ACCATGCCCC AGATTCAGGA TCAGACACCT AGTCCTTCCG | |
| 4401 | ATGGAAGCAC TAGACAAAGT TCACCTGAGC CTAATAGTCC CAGTGAATAT | |
| 4451 | TGTTTTTATG TGGATAGTGA TATGGTCAAT GAATTCAAGT TGGAATTGGT | |
| 4501 | AGAAAAACTT TTTGCTGAAG ACACAGAAGC AAAGAACCCA TTTTCTACTC | |
| 4551 | AGGACACAGA TTTAGACTTG GAGATGTTAG CTCCCTATAT CCCAATGGAT | |
| 4601 | GATGACTTCC AGTTACGTTC CTTCGATCAG TTGTCACCAT TAGAAAGCAG | |
| 4651 | TTCCGCAAGC CCTGAAAGCG CAAGTCCTCA AAGCACAGTT ACAGTATTCC | |
| 4701 | AGCAGACTCA AATACAAGAA CCTACTGCTA ATGCCACCAC TACCACTGCC | |
| 4751 | ACCACTGATG AATTAAAAAC AGTGACAAAA GACCGTATGG AAGACATTAA | |
| 4801 | AATATTGATT GCATCTCCAT CTCCTACCCA CATACATAAA GAAACTACTA | |
| 4851 | GTGCCACATC ATCACCATAT AGAGATACTC AAAGTCGGAC AGCCTCACCA | |
| 4901 | AACAGAGCAG GAAAAGGAGT CATAGAACAG ACAGAAAAAT CTCATCCAAG | |
| 4951 | AAGCCCTAAC GTGTTATCTG TCGCTTTGAG TCAAAGAACT ACAGTTCCTG | |
| 5001 | AGGAAGAACT AAATCCAAAG ATACTAGCTT TGCAGAATGC TCAGAGAAAG | |
| 5051 | CGAAAAATGG AACATGATGG TTCACTTTTT CAAGCAGTAG GAATTGGAAC | |
| 5101 | ATTATTACAG CAGCCAGACG ATCATGCAGC TACTACATCA CTTTCTTGGA | |
| 5151 | AACGTGTAAA AGGATGCAAA TCTAGTGAAC AGAATGGAAT GGAGCAAAAG | |
| 5201 | ACAATTATTT TAATACCCTC TGATTTAGCA TGTAGACTGC TGGGGCAATC | |
| 5251 | AATGGATGAA AGTGGATTAC CACAGCTGAC CAGTTATGAT TGTGAAGTTA | |
| 5301 | ATGCTCCTAT ACAAGGCAGC AGAAACCTAC TGCAGGGTGA AGAATTACTC | |
| 5351 | AGAGCTTTGG ATCAAGTTAA CTGAGCGGAT CCGACGGGGA TCCTCTAGCG | |
| 5401 | TTATCCATCA CACTGGCGGC CGCGACTCTA GAGTCGACCT CGAGGGGGGG | |
| 5451 | CCCGGACCTA CTAGGGTGCT GTGGAAGGGT GATGGTGCAG TAGTAGTTAA | |
| 5501 | TGATGAAGGA AAGGCAATAA TTGCTGTACC ATTAACCAGG ACTAAGTTAC | |
| 5551 | TAATAAAACC AAATTGAGTA TTGTTGCAGG AAGCAAGACC CAACTACCAT | |
| 5601 | TGTCAGCTGT GTTTCCTGAC CTCAATATTT GTTATAAGGT TTGATATGAA | |
| 5651 | TCCCAGGGGG AATCTCAACC CCTATTACCC AACAGTCAGA AAAATCTAAG | |
| 5701 | TGTGAGGAGA ACACAATGTT TCAACCTTAT TGTTATAATA ATGACAGTAA | |
| 5751 | GAACAGCATG GCAGAATCGA AGGAAGCAAG AGACCAAGAA TGAACCTGAA | |
| 5801 | AGAAGAATCT AAAGAAGAAA AAAGAAGAAA TGACTGGTGG AAAATAGGTA | |
| 5851 | TGTTTCTGTT ATGCTTAGCA GGAACTACTG GAGGAATACT TTGGTGGTAT | |
| 5901 | GAAGGACTCC CACAGCAACA TTATATAGGG TTGGTGGCGA TAGGGGGAAG | |
| 5951 | ATTAAACGGA TCTGGCCAAT CAAATGCTAT AGAATGCTGG GGTTCCTTCC | |
| 6001 | CGGGGTGTAG ACCATTTCAA AATTACTTCA GTTATGAGAC CAATAGAAGC | |
| 6051 | ATGCATATGG ATAATAATAC TGCTACATTA TTAGAAGCTT TAACCAATAT | |
| 6101 | AACTGCTCTA TAAATAACAA AACAGAATTA GAAACATGGA AGTTAGTAAA | |
| 6151 | GACTTCTGGC ATAACTCCTT TACCTATTTC TTCTGAAGCT AACACTGGAC | |
| 6201 | TAATTAGACA TAAGAGAGAT TTTGGTATAA GTGCAATAGT GGCAGCTATT | |
| 6251 | GTAGCCGCTA CTGCTATTGC TGCTAGCGCT ACTATGTCTT ATGTTCCTCT | |
| 6301 | AACTGAGGTT AACAAAATAA TGGAAGTACA AAATCATACT TTTGAGGTAG | |
| 6351 | AAAATAGTAC TCTAAATGGT ATGGATTTAA TAGAACGACA AATAAAGATA | |
| 6401 | TTATATGCTA TGATTCTTCA AACACATGCA GATGTTCAAC TGTTAAAGGA | |
| 6451 | AAGACAACAG GTAGAGGAGA CATTTAATTT AATTGGATGT ATAGAAAGAA | |
| 6501 | CACATGTATT TTGTCATACT GGTCATCCCT GGAATATGTC ATGGGGACAT | |
| 6551 | TTAAATGAGT CAACACAATG GGATGACTGG GTAAGCAAAA TGGAAGATTT | |
| 6601 | AAATCAAGAG ATACTAACTA CACTTCATGG AGCCAGGAAC AATTTGGCAC | |
| 6651 | AATCCATGAT AACATTCAAT ACACCAGATA GTATAGCTCA ATTTGGAAAA | |
| 6701 | GACCTTTGGA GTCATATTGG AAATTGGATT CCTGGATTGG GAGCTTCCAT | |
| 6751 | TATAAAATAT ATAGTGATGT TTTTGCTTAT TTATTTGTTA CTAACCTCTT | |
| 6801 | CGCCTAAGAT CCTCAGGGCC CTCTGGAAGG TGACCAGTGG TGCAGGGTCC | |
| 6851 | TCCGGCAGTC GTTACCTGAA GAAAAAATTC CATCACAAAC ATGCATCGCG | |
| 6901 | AGAAGACACC TGGGACCAGG CCCAACACAA CATACACCTA GCAGGCGTGA | |
| 6951 | CCGGTGGATC AGGGGACAAA TACTACAAGC AGAAGTACTC CAGGAACGAC | |
| 7001 | TGGAATGGAG AATCAGAGGA GTACAACAGG CGGCCAAAGA GCTGGGTGAA | |
| 7051 | GTCAATCGAG GCATTTGGAG AGAGCTATAT TTCCGAGAAG ACCAAAGGGG | |
| 7101 | AGATTTCTCA GCCTGGGGCG GCTATCAACG AGCACAAGAA CGGCTCTGGG | |
| 7151 | GGGAACAATC CTCACCAAGG GTCCTTAGAC CTGGAGATTC GAAGCGAAGG | |
| 7201 | AGGAAACATT TATGACTGTT GCATTAAAGC CCAAGAAGGA ACTCTCGCTA | |
| 7251 | TCCCTTGCTG TGGATTTCCC TTATGGCTAT TTTGGGGACT AGTAATTATA | |
| 7301 | GTAGGACGCA TAGCAGGCTA TGGATTACGT GGACTCGCTG TTATAATAAG | |
| 7351 | GATTTGTATT AGAGGCTTAA ATTTGATATT TGAAATAATC AGAAAAATGC | |
| 7401 | TTGATTATAT TGGAAGAGCT TTAAATCCTG GCACATCTCA TGTATCAATG | |
| 7451 | CCTCAGTATG TTTAGAAAAA CAAGGGGGGA ACTGTGGGGT TTTTATGAGG | |
| 7501 | GGTTTTATAA ATGATTATAA GAGTAAAAAG AAAGTTGCTG ATGCTCTCAT | |
| 7551 | AACCTTGTAT AACCCAAAGG ACTAGCTCAT GTTGCTAGGC AACTAAACCG | |
| 7601 | CAATAACCGC ATTTGTGACG CGAGTTCCCC ATTGGTGACG CGTTAACTTC | |
| 7651 | CTGTTTTTAC AGTATATAAG TGCTTGTATT CTGACAATTG GGCACTCAGA | |
| 7701 | TTCTGCGGTC TGAGTCCCTT CTCTGCTGGG CTGAAAAGGC CTTTGTAATA | |
| 7751 | AATATAATTC TCTACTCAGT CCCTGTCTCT AGTTTGTCTG TTCGAGATCC | |
| 7801 | TACAGAGCTC ATGCCTTGGC GTAATCATGG TCATAGCTGT TTCCTGTGTG | |
| 7851 | AAATTGTTAT CCGCTCACAA TTCCACACAA CATACGAGCC GGAAGCATAA | |
| 7901 | AGTGTAAAGC CTGGGGTGCC TAATGAGTGA GCTAACTCAC ATTAATTGCG | |
| 7951 | TTGCGCTCAC TGCCCGCTTT CCAGTCGGGA AACCTGTCGT GCCAGCTGCA | |
| 8001 | TTAATGAATC GGCCAACGCG CGGGGAGAGG CGGTTTGCGT ATTGGGCGCT | |
| 8051 | CTTCCGCTTC CTCGCTCACT GACTCGCTGC GCTCGGTCGT TCGGCTGCGG | |
| 8101 | CGAGCGGTAT CAGCTCACTC AAAGGCGGTA ATACGGTTAT CCACAGAATC | |
| 8151 | AGGGGATAAC GCAGGAAAGA ACATGTGAGC AAAAGGCCAG CAAAAGGCCA | |
| 8201 | GGAACCGTAA AAAGGCCGCG TTGCTGGCGT TTTTCCATAG GCTCCGCCCC | |
| 8251 | CCTGACGAGC ATCACAAAAA TCGACGCTCA AGTCAGAGGT GGCGAAACCC | |
| 8301 | GACAGGACTA TAAAGATACC AGGCGTTTCC CCCTGGAAGC TCCCTCGTGC | |
| 8351 | GCTCTCCTGT TCCGACCCTG CCGCTTACCG GATACCTGTC CGCCTTTCTC | |
| 8401 | CCTTCGGGAA GCGTGGCGCT TTCTCATAGC TCACGCTGTA GGTATCTCAG | |
| 8451 | TTCGGTGTAG GTCGTTCGCT CCAAGCTGGG CTGTGTGCAC GAACCCCCCG | |
| 8501 | TTCAGCCCGA CCGCTGCGCC TTATCCGGTA ACTATCGTCT TGAGTCCAAC | |
| 8551 | CCGGTAAGAC ACGACTTATC GCCACTGGCA GCAGCCACTG GTAACAGGAT | |
| 8601 | TAGCAGAGCG AGGTATGTAG GCGGTGCTAC AGAGTTCTTG AAGTGGTGGC | |
| 8651 | CTAACTACGG CTACACTAGA AGGACAGTAT TTGGTATCTG CGCTCTGCTG | |
| 8701 | AAGCCAGTTA CCTTCGGAAA AAGAGTTGGT AGCTCTTGAT CCGGCAAACA | |
| 8751 | AACCACCGCT GGTAGCGGTG GTTTTTTTGT TTGCAAGCAG CAGATTACGC | |
| 8801 | GCAGAAAAAA AGGATCTCAA GAAGATCCTT TGATCTTTTC TACGGGGTCT | |
| 8851 | GACGCTCAGT GGAACGAAAA CTCACGTTAA GGGATTTTGG TCATGAGATT | |
| 8901 | ATCAAAAAGG ATCTTCACCT AGATCCTTTT AAATTAAAAA TGAAGTTTTA | |
| 8951 | AATCAATCTA AAGTATATAT GAGTAAACTT GGTCTGACAG TTACCAATGC | |
| 9001 | TTAATCAGTG AGGCACCTAT CTCAGCGATC TGTCTATTTC GTTCATCCAT | |
| 9051 | AGTTGCCTGA CTCCCCGTCG TGTAGATAAC TACGATACGG GAGGGCTTAC | |
| 9101 | CATCTGGCCC CAGTGCTGCA ATGATACCGC GAGACCCACG CTCACCGGCT | |
| 9151 | CCAGATTTAT CAGCAATAAA CCAGCCAGCC GGAAGGGCCG AGCGCAGAAG | |
| 9201 | TGGTCCTGCA ACTTTATCCG CCTCCATCCA GTCTATTAAT TGTTGCCGGG | |
| 9251 | AAGCTAGAGT AAGTAGTTCG CCAGTTAATA GTTTGCGCAA CGTTGTTGCC | |
| 9301 | ATTGCTACAG GCATCGTGGT GTCACGCTCG TCGTTTGGTA TGGCTTCATT | |
| 9351 | CAGCTCCGGT TCCCAACGAT CAAGGCGAGT TACATGATCC CCCATGTTGT | |
| 9401 | GCAAAAAAGC GGTTAGCTCC TTCGGTCCTC CGATCGTTGT CAGAAGTAAG | |
| 9451 | TTGGCCGCAG TGTTATCACT CATGGTTATG GCAGCACTGC ATAATTCTCT | |
| 9501 | TACTGTCATG CCATCCGTAA GATGCTTTTC TGTGACTGGT GAGTACTCAA | |
| 9551 | CCAAGTCATT CTGAGAATAG TGTATGCGGC GACCGAGTTG CTCTTGCCCG | |
| 9601 | GCGTCAATAC GGGATAATAC CGCGCCACAT AGCAGAACTT TAAAAGTGCT | |
| 9651 | CATCATTGGA AAACGTTCTT CGGGGCGAAA ACTCTCAAGG ATCTTACCGC | |
| 9701 | TGTTGAGATC CAGTTCGATG TAACCCACTC GTGCACCCAA CTGATCTTCA | |
| 9751 | GCATCTTTTA CTTTCACCAG CGTTTCTGGG TGAGCAAAAA CAGGAAGGCA | |
| 9801 | AAATGCCGCA AAAAAGGGAA TAAGGGCGAC ACGGAAATGT TGAATACTCA | |
| 9851 | TACTCTTCCT TTTTCAATAT TATTGAAGCA TTTATCAGGG TTATTGTCTC | |
| 9901 | ATGAGCGGAT ACATATTTGA ATGTATTTAG AAAAATAAAC AAATAGGGGT | |
| 9951 | TCCGCGCACA TTTCCCCGAA AAGTGCCACC TAAATTGTAA GCGTTAATAT | |
| 10001 | TTTGTTAAAA TTCGCGTTAA ATTTTTGTTA AATCAGCTCA TTTTTTAACC | |
| 10051 | AATAGGCCGA AATCGGCAAA ATCCCTTATA AATCAAAAGA ATAGACCGAG | |
| 10101 | ATAGGGTTGA GTGTTGTTCC AGTTTGGAAC AAGAGTCCAC TATTAAAGAA | |
| 10151 | CGTGGACTCC AACGTCAAAG GGCGAAAAAC CGTCTATCAG GGCGATGGCC | |
| 10201 | CACTACGTGA ACCATCACCC TAATCAAGTT TTTTGGGGTC GAGGTGCCGT | |
| 10251 | AAAGCACTAA ATCGGAACCC TAAAGGGAGC CCCCGATTTA GAGCTTGACG | |
| 10301 | GGGAAAGCCA ACCTGGCTTA TCGAAATTAA TACGACTCAC TATAGGGAGA | |
| 10351 | CCGGC | |

| The nucleotide sequence of pSMART CMV-empty | | |
|---|---|---|
| 1 | AGATCTTGAA TAATAAAATG TGTGTTTGTC CGAAATACGC GTTTTGAGAT | SEQ ID NO:5 |
| 51 | TTCTGTCGCC GACTAAATTC ATGTCGCGCG ATAGTGGTGT TTATCGCCGA | |
| 101 | TAGAGATGGC GATATTGGAA AAATTGATAT TTGAAAATAT GGCATATTGA | |
| 151 | AAATGTCGCC GATGTGAGTT TCTGTGTAAC TGATATCGCC ATTTTTCCAA | |
| 201 | AAGTGATTTT TGGGCATACG CGATATCTGG CGATAGCGCT TATATCGTTT | |
| 251 | ACGGGGGATG GCGATAGACG ACTTTGGTGA CTTGGGCGAT TCTGTGTGTC | |
| 301 | GCAAATATCG CAGTTTCGAT ATAGGTGACA GACGATATGA GGCTATATCG | |
| 351 | CCGATAGAGG CGACATCAAG CTGGCACATG GCCAATGCAT ATCGATCTAT | |
| 401 | ACATTGAATC AATATTGGCC ATTAGCCATA TTATTCATTG GTTATATAGC | |
| 451 | ATAAATCAAT ATTGGCTATT GGCCATTGCA TACGTTGTAT CCATATCGTA | |
| 501 | ATATGTACAT TTATATTGGC TCATGTCCAA CATTACCGCC ATGTTGACAT | |
| 551 | TGATTATTGA CTAGTTATTA ATAGTAATCA ATTACGGGGT CATTAGTTCA | |
| 601 | TAGCCCATAT ATGGAQTTCC GCGTTACATA ACTTACGGTA AATGGCCCGC | |
| 651 | CTGGCTGACC GCCCAACGAC CCCCGCCCAT TGACGTCAAT AATGACGTAT | |
| 701 | GTTCCCATAG TAACGCCAAT AGGGACTTTC CATTGACGTC AATGGGTCGA | |
| 751 | GTATTTACGG TAAACTGCCC ACTTGGCAGT ACATCAAGTG TATCATATGC | |
| 801 | CAAGTCCGCC CCCTATTGAC GTCAATGACG GTAAATGGCC CGCCTGGCAT | |
| 851 | TATGCCCAGT ACATGACCTT ACGGGACTTT CCTACTTGGC AGTACATCTA | |
| 901 | CGTATTAGTC ATCGCTATTA CCATGGTGAT GCGGTTTTGG CAGTACACCA | |
| 951 | ATGGGCGTGG ATAGCGGTTT GACTCACGGG GATTTCCAAG TCTCCACCCC | |
| 1001 | ATTGACGTCA ATGGGAGTTT GTTTTGGCAC CAAAATCAAC GGGACTTTCC | |
| 1051 | AAAATGTCGT AACAACTGCG ATCGCCCGCC CCGTTGACGC AAATGGGCGG | |
| 1101 | TAGGCGTGTA CGGTGGGAGG TCTATATAAG CAGAGCTCGT TTAGTGAACC | |
| 1151 | GGGCACTCAG ATTCTGCGGT CTGAGTCCCT TCTCTGCTGG GCTGAAAAGG | |
| 1201 | CCTTTGTAAT AAATATAATT CTCTACTCAG TCCCTGTCTC TAGTTTGTCT | |
| 1251 | GTTCGAGATC CTACAGTTGG CGCCCGAACA GCGACCTGAG AGGGGCGCAG | |
| 1301 | ACCCTACCTG TTGAACCTGG CTGATCGTAG GATCCCCGGG ACAGCAGAGG | |
| 1351 | AGAACTTACA GAAGTCTTCT GGAGGTGTTC CTGGCCAGAA CACAGGAGGA | |
| 1401 | CAGGTAAGAT TGGGAGACCC TTTGACATTG GAGCAAGGCG CTCAAGAACT | |
| 1451 | TAGAGAAGGT GACGGTACAA GGGTCTCAGA AATTAACTAC TGGTAACTGT | |
| 1501 | AATTGGGCGC TAAGTCTAGT AGACTTATTT CATGATACCA ACTTTGTAAA | |
| 1551 | AGAAAAGGAC TGGCAGCTGA GGGATGTCAT TCCATTGCTG GAAGATGTAA | |
| 1601 | CTCAGACGCT GTCAGGACAA GAAAGAGAGG CCTTTGAAAG AACATGGTGG | |
| 1651 | GCAATTTCTG CTGTAAAGAT GGGCCTCCAG ATTAATAATG TAGTAGATGG | |
| 1701 | AAAGGCATCA TTCCAGCTCC TAAGAGCGAA ATATGAAAAG AAGACTGCTA | |
| 1751 | ATAAAAAGCA GTCTGAGCCC TCTGAAGAAT ATCTCTAGAG TCGACGCTCT | |
| 1801 | CATTACTTGT AACAAAGGGA GGGAAAGTAT GGGAGGACAG ACACCATGGG | |
| 1851 | AAGTATTTAT CACTAATCAA GCACAAGTAA TACATGAGAA ACTTTTACTA | |
| 1901 | CAGCAAGCAC AATCCTCCAA AAAATTTTGT TTTTACAAAA TCCCTGGTGA | |
| 1951 | ACATGGTCGA CTCTAGAACT AGTGGATCCC CCGGGCTGCA GGAGTGGGGA | |
| 2001 | GGCACGATGG CCGCTTTGGT CGAGGCGGAT CCGGCCATTA GCCATATTAT | |
| 2051 | TCATTGGTTA TATAGCATAA ATCAATATTG GCTATTGGCC ATTGCATACG | |
| 2101 | TTGTATCCAT ATCATAATAT GTACATTTAT ATTGGCTCAT GTCCAACATT | |
| 2151 | ACCGCCATGT TGACATTGAT TATTGACTAG TTATTAATAG TAATCAATTA | |
| 2201 | CGGGGTCATT AGTTCATAGC CCATATATGG AGTTCCGCGT TACATAACTT | |
| 2251 | ACGGTAAATG GCCCGCCTGG CTGACCGCCC AACGACCCCC GCCCATTGAC | |
| 2301 | GTCAATAATG ACGTATGTTC CCATAGTAAC GCCAATAGGG ACTTTCCATT | |
| 2351 | GACGTCAATG GGTGGAGTAT TTACGGTAAA CTGCCCACTT GGCAGTACAT | |
| 2401 | CAAGTGTATC ATATGCCAAG TACGCCCCCT ATTGACGTCA ATGACGGTAA | |
| 2451 | ATGGCCCGCC TGGCATTATG CCCAGTACAT GACCTTATGG GACTTTCCTA | |
| 2501 | CTTGGCAGTA CATCTACGTA TTAGTCATCG CTATTACCAT GGTGATGCGG | |
| 2551 | TTTTGGCAGT ACATCAATGG GCGTGGATAG CGGTTTGACT CACGGGGATT | |
| 2601 | TCCAAGTCTC CACCCCATTG ACGTCAATGG GAGTTTGTTT TGGCACCAAA | |
| 2651 | ATCAACGGGA CTTTCCAAAA TGTCGTAACA ACTCCGCCCC ATTGACGCAA | |
| 2701 | ATGGGCGGTA GGCATGTACG GTGGGAGGTC TATATAAGCA GAGCTCGTTT | |
| 2751 | AGTGAACCGT CAGATCGCCT GGCCGCGACT CTAGAGTCGA CCTCGAGGGG | |
| 2801 | GGGCCCGGAC CTACTAGGGT GCTGTGGAAG GGTGATGGTG CAGTAGTAGT | |
| 2851 | TAATGATGAA GGAAAGGGAA TAATTGCTGT ACCATTAACC AGGACTAAGT | |
| 2901 | TACTAATAAA ACCAAATTGA GTATTGTTGC AGGAAGCAAG ACCCAACTAC | |
| 2951 | CATTGTCAGC TGTGTTTCCT GACCTCAATA TTTGTTATAA GGTTTGATAT | |
| 3001 | GAATCCCAGG GGGAATCTCA ACCCCTATTA CCCAACAGTC AGAAAAATCT | |
| 3051 | AAGTGTGAGG AGAACACAAT GTTTCAACCT TATTGTTATA ATAATGACAG | |
| 3101 | TAAGAACAGC ATGGCAGAAT CGAAGGAAGC AAGAGACCAA GAATGAACCT | |
| 3151 | GAAAGAAGAA TCTAAAGAAG AAAAAAGAAG AAATGACTGG TGGAAAATAG | |
| 3201 | GTATGTTTCT GTTATGCTTA GCAGGAACTA CTGGAGGAAT ACTTTGGTGG | |
| 3251 | TATGAAGGAC TCCCACAGCA ACATTATATA GGGTTGGTGG CGATAGGGGG | |
| 3301 | AAGATTAAAC GGATCTGGCC AATCAAATGC TATAGAATGC TGGGGTTCCT | |
| 3351 | TCCCCGGGTG TAGACCATTT CAAAATTACT TCAGTTATGA GACCAATAGA | |
| 3401 | AGCATGCATA TGGATAATAA TACTGCTACA TTATTAGAAG CTTTAACCAA | |
| 3451 | TATAACTGCT CTATAAATAA CAAAACAGAA TTAGAAACAT GGAAGTTAGT | |
| 3501 | AAAGACTTCT GGCATAACTC CTTTACCTAT TTCTTCTGAA GCTAACACTG | |
| 3551 | GACTAATTAG ACATAAGAGA GATTTTGGTA TAAGTGCAAT AGTGGCAGCT | |
| 3601 | ATTGTAGCCG CTACTGCTAT TGCTGCTAGC GCTACTATGT CTTATGTTGC | |
| 3651 | TCTAACTGAG GTTAACAAAA TAATGGAAGT ACAAAATCAT ACTTTTGAGG | |
| 3701 | TAGAAAATAG TACTCTAAAT GGTATGGATT TAATAGAACG ACAAATAAAG | |
| 3751 | ATATTATATG CTATGATTCT TCAAACACAT GCAGATGTTC AACTGTTAAA | |
| 3801 | GGAAAGACAA CAGGTAGAGG AGACATTTAA TTTAATTGGA TGTATAGAAA | |
| 3851 | GAACACATGT ATTTTGTCAT ACTGGTCATC CCTGGAATAT GTCATGGGGA | |
| 3901 | CATTTAAATG AGTCAACACA ATGGGATGAC TGGGTAAGCA AAATGGAAGA | |
| 3951 | TTTAAATCAA GACATACTAA CTACACTTCA TGGAGCCAGG AACAATTTGG | |
| 4001 | CACAATCCAT GATAACATTC AATACACCAG ATAGTATAGC TCAATTTGGA | |
| 4051 | AAAGACCTTT GGAGTCATAT TGGAAATTGG ATTCCTGGAT TGGGAGCTTC | |
| 4101 | CATTATAAAA TATATAGTGA TGTTTTTGCT TATTTATTTG TTACTAACCT | |
| 4151 | CTTCGCCTAA GATCCTCAGG GCCCTCTGGA AGGTGACCAG TGGTGCAGGG | |
| 4201 | TCCTCCGGCA GTCGTTACCT GAAGAAAAAA TTCCATCACA AACATGCATC | |
| 4251 | GCGAGAAGAC ACCTGGGACC AGGCCCAACA CAACATACAC CTAGCAGGCG | |
| 4301 | TGACCGGTGG ATCAGGGGAC AAATACTACA AGCAGAAGTA CTCCAGGAAC | |
| 4351 | GACTGGAATG GAGAATCAGA GGAGTACAAC AGGCGGCCAA AGAGCTGGGT | |
| 4401 | GAAGTCAATC GAGGCATTTG GAGAGAGCTA TATTTCCGAG AAGACCAAAG | |
| 4451 | GGGAGATTTC TCAGCCTGGG GCGGCTATCA ACGAGCACAA GAACGGCTCT | |
| 4501 | GGGGGGAACA ATCCTCACCA AGGGTCCTTA GACCTGGAGA TTCGAAGCGA | |
| 4551 | AGGAGGAAAC ATTTATGACT GTTGCATTAA AGCCCAAGAA GGAACTCTCG | |
| 4601 | CTATCCCTTG CTGTGGATTT CCCTTATGGC TATTTTGGGG ACTAGTAATT | |
| 4651 | ATAGTAGGAC GCATAGCAGG CTATGGATTA CGTGGACTCG CTGTTATAAT | |
| 4701 | AAGGATTTGT ATTAGAGGCT TAAATTTGAT ATTTGAAATA ATCAGAAAAA | |
| 4751 | TGCTTGATTA TATTGGAAGA GCTTTAAATC CTGGCACATC TCATGTATCA | |
| 4801 | ATGCCTCAGT ATGTTTAGAA AAACAACGGG GGAACTGTGG GGTTTTTATG | |
| 4851 | ACGGGTTTTA TAAATGATTA TAAGAGTAAA AAGAAAGTTG CTGATGCTCT | |
| 4901 | CATAACCTTG TATAACCCAA AGGACTAGCT CATGTTGCTA GGCAACTAAA | |
| 4951 | CCGCAATAAC CGCATTTGTG ACGCGAGTTC CCCATTGGTG ACGCGTTAAC | |
| 5001 | TTCCTGTTTT TACAGTATAT AAGTGCTTGT ATTCTGACAA TTGGGCACTC | |
| 5051 | AGATTCTGCG GTCTGAGTCC CTTCTCTGCT GGGCTGAAAA GGCCTTTGTA | |
| 5101 | ATAAATATAA TTCTCTACTC AGTCCCTGTC TCTAGTTTGT CTGTTCGAGA | |
| 5151 | TCCTACAGAG CTCATGCCTT GGCGTAATCA TGGTCATAGC TGTTTCCTGT | |
| 5201 | GTGAAATTGT TATCCGCTCA CAATTCCACA CAACATACGA GCCGGAAGCA | |
| 5251 | TAAAGTGTAA AGCCTGGGGT GCCTAATGAG TGAGCTAACT CACATTAATT | |
| 5301 | GCGTTGCGCT CACTGCCCGC TTTCCAGTCG GGAAACCTGT CGTGCCAGCT | |
| 5351 | GCATTAATGA ATCGGCCAAC GCGCGGGGAG AGGCGGTTTG CGTATTGGGC | |
| 5401 | GCTCTTCCGC TTCCTCGCTC ACTGACTCGC TGCGCTCGGT CGTTCGGCTG | |
| 5451 | CGGCGAGCGG TATCAGCTCA CTCAAAGGCG GTAATACGGT TATCCACAGA | |
| 5501 | ATCAGGGGAT AACGCAGGAA AGAACATGTG AGCAAAAGGC CAGCAAAAGG | |
| 5551 | CCAGGAACCG TAAAAAGGCC GCGTTGCTGG CGTTTTTCCA TAGGCTCCGC | |
| 5601 | CCCCCTGACG AGCATCACAA AAATCGACGC TCAAGTCAGA GGTGGCGAAA | |
| 5651 | CCCGACAGGA CTATAAAGAT ACCAGGCGTT TCCCCCTGGA AGCTCCCTCG | |
| 5701 | TGCGCTCTCC TGTTCCGACC CTGCCGCTTA CCGGATACCT GTCCGCCTTT | |
| 5751 | CTCCCTTCGG GAAGCGTGGC GCTTTCTCAT AGCTCACGCT GTAGGTATCT | |
| 5801 | CAGTTCGGTG TAGGTCGTTC GCTCCAAGCT GGGCTGTGTG CACGAACCCC | |
| 5851 | CCGTTCAGCC CGACCGCTGC GCCTTATCCG GTAACTATCG TCTTGAGTCC | |
| 5901 | AACCCGGTAA GACACGACTT ATCGCCACTG GCAGCAGCCA CTGGTAACAG | |
| 5951 | GATTAGCAGA GCGAGGTATG TAGGCGGTGC TACAGAGTTC TTGAAGTGGT | |
| 6001 | GGCCTAACTA CGGCTACACT AGAAGGACAG TATTTGGTAT CTGCGCTCTG | |
| 6051 | CTGAAGCCAG TTACCTTCGG AAAAAGAGTT GGTAGCTCTT GATCCGGCAA | |
| 6101 | ACAAACCACC GCTGGTAGCG GTGGTTTTTT TGTTTGCAAG CAGGAGATTA | |
| 6151 | CGCGCAGAAA AAAAGGATCT CAAGAAGATC CTTTGATCTT TTCTACGGGG | |
| 6201 | TCTGACGCTC AGTGGAACGA AAACTCACGT TAAGGGATTT TGGTCATGAG | |
| 6251 | ATTATCAAAA AGGATCTTCA CCTAGATCCT TTTAAATTAA AAATGAAGTT | |
| 6301 | TTAAATCAAT CTAAAGTATA TATGAGTAAA CTTGGTCTGA CAGTTACCAA | |
| 6351 | TGCTTAATCA GTGAGGCACC TATCTCAGCG ATCTGTCTAT TTCGTTCATC | |
| 6401 | CATAGTTGCC TGACTCCCCG TCGTGTAGAT AACTACGATA CGGGAGGGCT | |
| 6451 | TACCATCTGG CCCCAGTGCT GCAATGATAC CGCGAGACCC ACGCTCACCG | |
| 6501 | GCTCCAGATT TATCAGCAAT AAACCAGCCA GCCGGAAGGG CCGAGCGCAG | |
| 6551 | AAGTGGTCCT GCAACTTTAT CCGCCTCCAT CCAGTCTATT AATTGTTGCC | |
| 6601 | GGGAAGCTAG AGTAAGTAGT TCGCCAGTTA ATAGTTTGCG CAACGTTGTT | |
| 6651 | GCCATTGCTA CAGGCATCGT GGTGTCACGC TCGTCGTTTG GTATGGCTTC | |
| 6701 | ATTCAGCTCC GGTTCCCAAC GATCAAGGCG AGTTACATGA TCCCCCATGT | |
| 6751 | TGTGCAAAAA AGCGGTTAGC TCCTTCGGTC CTCCGATCGT TGTCAGAAGT | |
| 6801 | AAGTTGGCCG CAGTGTTATC ACTCATGGTT ATGGCAGCAC TGCATAATTC | |
| 6851 | TCTTACTGTC ATGCCATCCG TAAGATGCTT TTCTGTGACT GGTGAGTACT | |
| 6901 | CAACCAAGTC ATTCTGAGAA TAGTGTATGC GGCGACCGAG TTGCTCTTGC | |
| 6951 | CCGGCGTCAA TACGGGATAA TACCGCGCCA CATAGCAGAA CTTTAAAAGT | |
| 7001 | GCTCATCATT GGAAAACGTT CTTCGGGGCG AAAACTCTCA AGGATCTTAC | |
| 7051 | CGCTGTTGAG ATCCAGTTCG ATGTAACCCA CTCGTGCACC CAACTGATCT | |
| 7101 | TCAGCATCTT TTACTTTCAC CAGCGTTTCT GGGTGAGCAA AAACAGGAAG | |
| 7151 | GCAAAATGCC GCAAAAAAGG GAATAAGGGC GACACGGAAA TGTTGAATAC | |
| 7201 | TCATACTCTT CCTTTTTCAA TATTATTGAA GCATTTATCA GGGTTATTGT | |
| 7251 | CTCATGAGCG GATACATATT TGAATGTATT TAGAAAAATA AACAAATAGG | |
| 7301 | GGTTCCGCGC ACATTTCCCC GAAAAGTGCC ACCTAAATTG TAAGCGTTAA | |
| 7351 | TATTTTGTTA AAATTCGCGT TAAATTTTTG TTAAATCAGC TCATTTTTTA | |
| 7401 | ACCAATAGGC CGAAATCGGC AAAATCCCTT ATAAATCAAA AGAATAGACC | |
| 7451 | GAGATAGGGT TGAGTGTTGT TCCAGTTTGG AACAAGAGTC CACTATTAAA | |
| 7501 | GAACGTGGAC TCCAACGTCA AAGGGCGAAA AACCGTCTAT CAGGGCGATG | |
| 7551 | GCCCACTACG TGAACCATCA CCCTAATCAA GTTTTTTGGG GTCGAGGTGC | |
| 7601 | CGTAAAGCAC TAAATCGGAA CCCTAAAGGG AGCCCCCGAT TTAGAGCTTG | |
| 7651 | ACGGGGAAAG CCAACCTGGC TTATCGAAAT TAATACGACT CACTATAGGG | |
| 7701 | AGACCGGC | |

1. A differential expression screening method for identifying a genetic element involved in a cellular process, which method comprises:
comparing:
(a) gene expression in a first cell of interest; and
(b) gene expression in a second cell of interest, which cell comprises altered levels, relative to physiological levels, of a biological molecule implicated in the cellular process, due to the introduction into the second cell of a heterologous nucleic acid directing expression of a polypeptide; and
identifying a genetic element whose expression differs, wherein gene expression in the first and/or second cell of interest is compared under at least two different environmental conditions relevant to the cellular process.

2. The method of claim 1, wherein gene expression is compared in both the first and the second cell of interest under at least two different environmental conditions relevant to the cellular process.

3. The method of claim 1, wherein the method comprises:
comparing:
(a) gene expression in a first cell of interest;
(b) gene expression in the first cell of interest which has been exposed to an environmental change of a first type;
(c) gene expression in the first cell of interest which has been exposed to an environmental change of a second type; and
(d) gene expression in a second cell of interest, which cell contains altered levels, relative to physiological levels, of a biological molecule whose activity is responsive to one or both of the environmental changes recited in parts b) and c), due to the introduction into the second cell of a heterologous nucleic acid directing expression of a polypeptide, under conditions in which the cell either has or has not been exposed to the first and/or the second type of environmental change; and
identifying a genetic element whose expression differs.

4. The method of claim 1, wherein the different environmental conditions are different levels of a biological signal.

5. The method of claim 4, wherein the method comprises:
comparing:
(a) gene expression in a first cell of interest;
(b) gene expression in the first cell of interest which has been exposed to a biological signal relevant to the cellular process, wherein the biological signal is at a first level;
(c) gene expression in the first cell of interest which has been exposed to a biological signal relevant to the cellular process, wherein the biological signal is at a second level; and
(d) gene expression in a second cell of interest, which cell comprises altered levels, relative to physiological levels, of a biological molecule whose activity is responsive to the biological signal, due to the introduction into the second cell of a heterologous nucleic acid directing expression of a polypeptide, wherein the signal is absent, at a first level or at a second level; and
identifying a genetic element whose expression differs.

6. The method of claim 4, wherein the method comprises:
comparing:
(a) gene expression in a first cell of interest;
(b) gene expression in the first cell of interest, wherein the cell has been exposed to a biological signal relevant to the cellular process;
(c) gene expression in the first cell of interest, which cell contains altered levels, relative to physiological levels, of a biological molecule whose activity is responsive to the biological signal, due to the introduction into the first cell of a heterologous nucleic acid directing expression of a polypeptide, wherein the altered level of the biological molecule is at a first level, and wherein the biological signal is either present or absent;
(d) gene expression in a second cell of interest;
(e) gene expression in the second cell of interest, wherein the cell has been exposed to a biological signal relevant to the cellular process;
(f) gene expression in the second cell of interest, which cell contains altered levels, relative to physiological levels, of the biological molecule, due to the introduction into the second cell of a heterologous nucleic acid directing expression of the polypeptide, wherein the altered level of the biological molecule is at a second level, and wherein the biological signal is either present or absent; and
identifying a genetic element whose expression differs.

7. The method of claim 4, wherein the method comprises:
comparing:
(a) gene expression in a first cell of interest;
(b) gene expression in the first cell of interest, wherein the cell has been exposed to a biological signal relevant to the cellular process;
(c) gene expression in the first cell of interest, which cell contains altered levels, relative to physiological levels, of a first biological molecule whose activity is responsive to the biological signal, due to the introduction into the first cell of a heterologous nucleic acid directing expression of a first polypeptide, wherein the biological signal is either present or absent;
(d) gene expression in a second cell of interest;
(e) gene expression in the second cell of interest, wherein the cell has been exposed to a biological signal relevant to the cellular process;
(f) gene expression in the second cell of interest, which cell contains altered levels, relative to physiological levels, of a second biological molecule, due to the introduction into the second cell of a heterologous nucleic acid directing expression of a second polypeptide, wherein the biological signal is either present or absent; and
identifying a genetic element whose expression differs.

8. The method of claim 7, wherein the first polypeptide is HIF1-α, and the second polypeptide is EPAS1.

9. The method of claim 1, wherein the first and second cells are different cell types.

10. The method of claim 1, wherein the levels of the biological molecule are enhanced relative to physiological levels.

11. The method of claim 1, wherein the levels of the biological molecule are reduced relative to physiological levels.

12. The method of claim 1, wherein the biological molecule and the polypeptide are the same.

13. The method of claim 1, wherein the heterologous nucleic acid is introduced into the cell by means of a viral vector.

14. The method of claim 13, wherein the viral vector is a retrovirus, lentivirus (such as the Equine Infectious Anaemia Virus (EIAV) or human immunodeficiency virus type 1 (HIV-1)), an adenovirus, an adeno-associated virus, a herpes virus or a pox virus (such as entomopox).

15. The method of claim 1, wherein gene expression is determined by a proteomic technique.

16. The method of claim 1, wherein gene expression is determined using a genomic or cDNA technique.

17. The method of claim 1, wherein the first cell of interest has normal physiological levels of the biological molecule.

18. The method of claim 1, wherein the polypeptide is involved in the cellular process.

19. The method of claim 1, wherein the first cell is from a normal patient and the second cell is from a diseased patient.

20. The method of claim 1, wherein the first cell is from a diseased patient and the second cell is from the same diseased patient.

21. The method of claim 1, wherein the genetic element is a gene, a gene product or a regulatory element.

22. The method of claim 1, wherein the heterologous nucleic acid encodes a biological molecule selected from the group consisting of: HIF1α, EPAS1, a membrane bound form of the IL5α receptor, a soluble form of an IL5α receptor, Bcl-2, Bcl-x, FasL, NGF, GDNF, heat shock proteins (HSPs), APP, Presenilin 1, Presenilin 2, α-synuclein, Tau, Parkin and ubiquitin.

23. A differential expression screening method for identifying a gene or gene product whose expression is regulated by a signal, which comprises:
comparing at two different levels of the signal:
(a) gene expression in a first cell of interest wherein the signal is at a first level; and
(b) gene expression in a second cell of interest which cell comprises altered levels, relative to physiological levels, of a biological molecule whose activity is responsive to the signal, due to the introduction into the second cell of a heterologous nucleic acid, wherein the signal is at a second level; and
identifying a gene or gene product whose expression differs.

24. The method of claim 23, wherein the first and second cells are different cell types.

25. The method of claim 23, wherein the levels of the biological molecule are enhanced relative to physiological levels.

26. The method of claim 23, wherein the levels of the biological molecule are reduced relative to physiological levels.

27. The method of claim 23 wherein the heterologous nucleic acid is introduced into the cell by means of a viral vector.

28. The method of claim 26, wherein the viral vector is a retrovirus, lentivirus (such as the Equine Infectious Anaemia Virus (EIAV) or human immunodeficiency virus type 1 (HIV-1)), an adenovirus, an adeno-associated virus, a herpes virus or a pox virus (such as entomopox).

29. The method of claim 23, wherein gene expression is determined by a proteomic technique.

30. The method of any one of claim 22, wherein gene expression is determined using a genomic or cDNA technique.

31. The method of claim 23, wherein the first cell of interest has normal physiological levels of the biological molecule.

32. The method of claim 23, wherein the first cell is from a normal patient and the second cell is from a diseased patient.

33. The method of any one of claims 22, wherein the first cell is from a diseased patient and the second cell is from the same diseased patient.

34. The method of claim 23, wherein the heterologous nucleic acid encodes a biological molecule selected from the group consisting of: HIF1α, EPAS1, a membrane bound form of the IL5α receptor, a soluble form of an IL5α receptor, Bcl-2, Bcl-x, FasL, NGF, GDNF, heat shock proteins (HSPs), APP, Presenilin 1, Presenilin 2, α-synuclein, Tau, Parkin and ubiquitin.

35. A differential expression screening method for identifying a gene product involved in a disease process, which comprises:
(i) comparing gene expression in:
(a) a first cell of interest; and
(b) a second cell of interest;
(ii) comparing gene expression in
(a) the first cell of interest; and
(b) a third cell of interest which cell comprises altered levels, relative to physiological levels, of a candidate gene product, due to the introduction into the first cell of a heterologous nucleic acid directing expression of the candidate gene product; and
(iii) selecting those candidate gene products which give rise to an alteration in the levels of expression of a second gene product in the third cell of interest relative to the first cell of interest, which second gene product also has altered levels of expression in the second cell of interest relative to the first cell of interest.

36. The method of claim 35, wherein the candidate gene product is a polypeptide.

37. The method of claim 35, wherein the comparison of gene expression is carried out by identifying, using nucleic acid techniques, those mRNA transcripts whose levels are altered between the first cell of interest and the second cell of interest, and between the first cell of interest and the third cell of interest.

38. The method of claim 35, wherein the comparison of gene expression is carried out by identifying, using protein analytical procedures, those polypeptides whose levels are altered between the first cell of interest and the second cell of interest, and between the first cell of interest and the third cell of interest.

39. The method of claim 35, wherein the gene product is regulated by a signal, and gene expression is compared in the cells at two different levels of the signal.

40. The method of claim 35, wherein the heterologous nucleic acid encodes a biological molecule selected from the group consisting of: HIF1α, EPAS1, a membrane bound form of the IL5α receptor, a soluble form of an IL5α receptor, Bcl-2, Bcl-x, FasL, NGF, GDNF, heat shock proteins (HSPs), APP, Presenilin 1, Presenilin 2, α-synuclein, Tau, Parkin and ubiquitin.

41. A method for increasing the sensitivity of a differential expression screening method in which gene expression of a first and a second cell of interest in response to two different levels of a signal are compared, which comprises introducing a heterologous nucleic acid into the first cell or the second cell to increase the level of a biological molecule which modulates the response of the cell to the signal.
